SEQUENCE LISTING (6 sequences)

SEQ ID NO: 1 - protein, 23 aa, artificial
  INF7 peptide derived from the N-terminus of haemophilus influenza haemagglutinin-2
GLFGAIAGFIENGWEGMIDGGGC

SEQ ID NO: 2 - protein, 30 aa, artificial sequence
  synthetic fusogenic peptide
WEAALAEALAEALAEHLAEALAEALEALAA

SEQ ID NO: 3 - protein, 30 aa, Artificial sequence
  synthetic fusogenic peptide
WEAKLAKALAKALAKHLAKALAKALKACEA

SEQ ID NO: 4 - protein, 10 aa, Artificial sequence
  HIV tat peptide sequence protein transduction domain
GRKKRRQRRR

SEQ ID NO: 5 - protein, 9 aa, Artificial sequence
  oligoarginine protein transduction domain
RRRRRRRRR

SEQ ID NO: 6 - protein, 5 aa, Artificial sequence
  oligohistidine oligopeptide
HHHHH

(19) United States
(12) Patent Application Publication
(10) Pub. No.: US 2011/0229528 A1
(43) Pub. Date: Sep. 22, 2011

(54) PEGYLATED POLYPLEXES FOR POLYNUCLEOTIDE DELIVERY

(75) Inventors: JANNI MIROSEVICH, TAMPA, FL (US); KEVIN N. SILL, TAMPA, FL (US); GREGOIRE CARDOEN, PHILADELPHIA, PA (US)

(73) Assignee: INTEZYNE TECHNOLOGIES, INCORPORATED, TAMPA, FL (US)

(21) Appl. No.: 13/047,733

(22) Filed: Mar. 14, 2011

Related U.S. Application Data
(60) Provisional application No. 61/313,229, filed on Mar. 12, 2010.

(51) Int. Cl. A61K 9/00; A61K 38/02; A61K 38/22; A61K 38/40; A61K 31/7088; A61K 31/713; A61K 48/00

(52) U.S. Cl. 424/400; 514/1.1; 514/9.6; 514/13.5; 514/44 R; 514/44 A

(57) ABSTRACT

The present invention provides polymers, compositions thereof, and polyplexes comprising said polymers. In particular, cationic polymers, pegylated versions thereof, and polynucleotide containing polyplexes comprising such polymers are provided. The invention further provides methods of using said polymers and polyplexes.

### CROSS-REFERENCE TO RELATED APPLICATIONS

The present application claim priority to U.S. provisional patent application Ser. No. 61/313,229, filed Mar. 12, 2010, the entirety of each of which is hereby incorporated herein by reference.

### FIELD OF THE INVENTION

The present invention relates to the field of polymer chemistry and more particularly to the formation of polynucleotide containing polyplexes and uses thereof.

### BACKGROUND OF THE INVENTION

The development of new therapeutic agents has dramatically improved the quality of life and survival rate of patients suffering from a variety of disorders. However, drug delivery innovations are needed to improve the success rate of these treatments. Specifically, delivery systems are still needed which effectively minimize premature excretion and/or metabolism of therapeutic agents and deliver these agents specifically to diseased cells, thereby reducing their potentially adverse effects to healthy cells. Rationally-designed, nanoscopic drug carriers, or “nanovectors,” offer a promising approach to achieving these goals due to their inherent ability to overcome many biological barriers.

### BRIEF DESCRIPTION OF THE DRAWINGS

FIG. 1: Gel Retardation of DNA Complexed with Polymers.

FIGS. 2A-B: Size Analysis of Polyplexes at Various N:P Ratios.

FIG. 3: Buffering Capacity of P(Asp-DET) Polymer.

FIG. 4: Gel Retardation of DNA Complexed with Non and post-PEG Polymers.

FIG. 5: Size Analysis of Polyplexes pre- and post-PEG.

FIG. 6: TEM of D/L Asp-DET/DNA polyplexes.

FIG. 7: Erythrocyte Aggregation Study of Polyplexes pre- and post-PEG.

FIG. 8: GFP and Luciferase Expression of HCT-116 Cells Transiently Transfected with D/L Asp-DET Polymers.

FIG. 9: GFP and Luciferase Expression of HCT-116 Cells Transiently Transfected with D/L Asp-DET Polymers.

FIG. 10: Localization of Fluorescently Labeled DNA Transfected with Cationic Polymers.

FIGS. 11A-C: In vivo Studies Using D/L Asp-DET Post-PEG Polymers.

FIG. 12: In vivo Studies Using D/L Asp-DET Post-PEG Polymers.

FIG. 13: Schematic of Polyplex Preparation

FIG. 14: Size Analysis of Polyplexes Non-and Post-PEG

FIG. 15: TEM of Poly(d/l Asp-DET)/DNA Polyplexes

FIG. 16: Schematic of Polyplex Salt/Stability Assays

FIG. 17: Salt Addition and Centrifugation Studies Using Non and Post-PEG Polyplexes

FIG. 18: Salt Addition and Centrifugation Studies Using Non and Post-PEG Polyplexes

FIG. 19: Salt Addition and Centrifugation Studies Using Non and Post-PEG Polyplexes

FIG. 20: Serum Addition and Centrifugation Studies Using Non and Post-PEG Polyplexes

FIG. 21: Luciferase Expression of Cells Transiently Transfected with Poly(d/l Asp-DET) Polymers, Non- and Post-PEG

FIG. 22: Titration Curves

FIG. 23: Complexation Studies Studies Using D/L Asp-DET

FIG. 24: Polyplex Phisiochemical Properties as a Function of N:P ratio

FIG. 25: DNAse Protection Assay

FIG. 26: Flow Cytometry Cellular Uptake Experiments

FIGS. 27A-B: Comparison of Polyplex and PEG-Polyplex DNA Complexation Ability

FIGS. 28A-B: Physiochemical Characterization and Comparison of Polyplexes and PEG-Polyplexes

FIGS. 29A-B: Morphologies of PEG-Polyplexes using either Diblock Polymers or Cationic Homopolymer

FIGS. 30A-B: Size Analysis and Morphologies of Polyplexes at Various N:P Ratios

FIGS. 31A-C: In vivo Studies Using D/L Asp-DET and various PEG Polymers

FIGS. 32A-B: Luciferase Expression of Colon Cancer Cells Transiently Transfected with Poly(D/L Asp-DET)/DNA Polyplexes and PEGPolyplexes

FIGS. 33A-B: Strategy to Attach Targeting Groups to PEG-Polyplexes

FIGS. 34A-B: Localization and Expression of Luciferase Plasmid DNA in Colon Cancer Cells Transfected with Copper Click Chemistry Created EGF Targeted PEG-Polyplexes

FIG. 35: Adverse effects of Copper Click Reagents on DNA

FIGS. 36A-B: Gel Retardation and Size Analysis of EGF Targeted PEG Polyplexes Created by Copper free Click Chemistry

FIGS. 37A-B: Morphologies and Salt stability of EGF Targeted PEG Polyplexes Created by Copper free Click Chemistry

FIG. 38: Localization of Fluorescently Labeled DNA in Colon Cancer Cells Transfected with Targeted EGF PEG-Polyplexes

FIG. 39: Expression of Luciferase Plasmid DNA in Colon Cancer Cells Transfected with Copper Free Click Chemistry Created EGF Targeted PEG-Polyplexes

FIG. 40: In vivo Studies Using EGF Targeted PEG-Polyplexes

FIGS. 41A-B: Comparison of DNA complexation of DNA using either PEI or Poly(D/L Asp-DET) Polymers

FIGS. 42A-B: Co-complexation of DNA using Linear PEI and Poly(D/LAsp-DET) Polymers

FIGS. 43A-B: Complexation Studies Studies Using Poly(D/L Glu-DET) Polymers

FIGS. 44A-B: Size Analysis and Morphologies of Poly(D/L Glu-DET)/DNA Polyplexes at Various N:P Ratios

FIG. 45: DNAse Protection Assay of Poly(D/L Glu-DET)/DNA Polyplexes

FIG. 46: Luciferase Expression of HCT-116 Cells Transiently Transfected with Poly(D/L Glu-DET) Polymers

FIGS. 47A-B: Comparison of Polyplex and PEG-Polyplex DNA Complexation Ability

FIGS. 48A-B: Comparison of Polyplex and PEG-Polyplex Size and Morphology

FIG. 49: Salt Stability Studies of Poly(D/L Glu-DET)/DNA Polyplexes

FIG. 50: Localization and Expression of Luciferase Plasmid DNA in Colon Cancer Cells Transfected with Poly(D/L Glu-DET) Polymers

### DETAILED DESCRIPTION OF CERTAIN EMBODIMENTS OF THE INVENTION

### 1. General Description

There are several key factors that limit the use of lipoplexes and polyplexes for in vivo gene delivery applications, particularly when systemic delivery is desired. These include instability of these electrostatic assemblies in high salt environments, irreversible protein binding to the complex that can alter their pharmacokinetic profile, and capture by RES due to excess positive charge. The covalent attachment of poly(ethylene glycol) (PEG) to gene carriers has been shown to address many of these limitations by sterically shielding the complex from unwanted cellular and protein interactions as well as imparting the inherent, stealth properties of PEG. MacLachlan and coworkers have demonstrated that PEG-lipid conjugates, used in conjunction with traditional lipids, can dramatically improve the stability and circulation half-life of DNA-loaded lipoplexes (J. Control. Release, 2006, 112, 280). Similarly, Kissel and coworkers have developed PEG-modified PEI polyplexes that showed enhanced circulation lifetimes when compared to unmodified PEI polyplexes (Pharm. Res., 2002, 19, 810).

PEG has also become a standard choice for the hydrophilic, corona-forming segment of block copolymer polyplexs, and numerous studies have confirmed its ability to reduce RES uptake of micellar delivery systems. See Kwon, G.; Suwa, S.; Yokoyama, M.; Okano, T.; Sakurai, Y.; Kataoka, K. J. Cont. Rel. 1994, 29, 17-23; Caliceti, P.; Veronese, F. M. Adv. Drug Del. Rev. 2003, 55, 1261-1277; Ichikawa, K.; Hikita, T.; Maeda, N.; Takeuchi, Y.; Namba, Y.; Oku, N. Bio. Pharm. Bull. 2004, 27, and 443-444. The ability to tailor PEG chain lengths offers numerous advantages in drug carrier design since studies have shown that circulation times and RES uptake are influenced by the length of the PEG block. In general, longer PEG chains lead to longer circulation times and enhanced stealth properties. In a systematic study of PEG-b-poly(lactic-co-glycolic acid) (PLGA) polyplexs with PEG molecular weights ranging from 5,000-20,000 Da, Langer and coworkers demonstrated that polyplexes coated with 20,000 Da PEG chains were the least susceptible to liver uptake. After 5 hours of circulation, less than 30% of the polyplexs had accumulated in the liver. See Gref, R.; Minamitake, Y.; Peracchia, M. T.; Trubetskoy, V.; Torchilin, V.; Langer, R. Science 1994, 263, 1600-1603.

Two other aspects of a gene delivery system must also be considered; the buffering capacity of the polycation and the intracellular release of the polynucleotide from the polymer.

The present invention describes the preparation of a polycation with suitable buffering capacity and morphology to allow for polynucleotide release, complexation of the polycation with the polynucleotide, and the subsequent attachment of PEG to the polyplex for in vivo administration. In certain aspects, the present invention provides a polycation which is comprised of a poly(amino acid) (PAA) backbone with amine containing side chain groups.

While the methods to influence secondary structure of poly(amino acids) have been known for some time, it is believed that poly(amino acid) copolymers possessing a random coil conformation are particularly useful for the complexing of polynucleotides when compared to similar copolymers possessing a helical segment. Without wishing to be bound to any particular theory, it is believed that a cationic poly(amino acid) copolymer having a random coil conformation and thereby increased mobility and degrees of freedom allows for more efficient electrostatic interactions with the anionic polynucleotide, while the relative rigidity and limited degrees of freedom associated with a cationic poly(amino acid) that possesses secondary structure results in less effective complexation of the polynucleotide.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “portion” or “block” refers to a repeating polymeric sequence of defined composition. A portion or a block may consist of a single monomer or may be comprise of on or more monomers, resulting in a “mixed block”.

One skilled in the art will recognize that a monomer repeat unit is defined by parentheses depicted around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the term “polycation” or “cationic polymer” may be used interchangeably and refer to a polymer possessing a plurality of ionic charges. In some embodiments polycation also refers to a polymer that possess a plurality of functional groups that can be protonated to obtain a plurality of ionic charges. For clarity, a polymer that contains a plurality of amine functional groups will be referred to as a polycation or a cationic polymer within this application.

In certain embodiments, a provided cation is suitable for polynucleotide encapsulation. As used herein, the term “polynucleotide” refers to DNA or RNA. In some embodiments, a polynucleotide is a short interfering RNA (siRNA), a microRNA (miRNA), a plasmid DNA (pDNA), a short hairpin RNA (shRNA), messanger RNA (mRNA), antisense RNA (asRNA), to name a few, and encompasses both the nucleotide sequence and any structural embodiments thereof, such as double stranded, single stranded, helical, hairpin, etc.

As used herein, the terms “polynucleotide-loaded” and “encapsulated,” and derivatives thereof, are used interchangeably. In accordance with the present invention, a “polynucleotide-loaded” polyplex refers to a polyplex having one or more polynucleotides situated within the core of the polyplex. This is also referred to as a polynucleotide being “encapsulated” within the polyplex.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit is in the L-configuration. In other embodiments, the amino acid units are a mixture of D and L configurations. Such poly(amino acids) include those having suitably protected functional groups. For example, amino acid monomers may have hydroxyl or amino moieties that are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In still other embodiments, amino acid blocks of the present invention include random amino acid blocks, i.e., blocks comprising a mixture of amino acid residues.

As used herein, the phrase “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the term “D,L-mixed poly(amino acid)” refers to a poly(amino acid) wherein the poly(amino acid) consists of a mixture of amino acids in both the D- and L-configurations. It is well established that homopolymers and copolymers of amino acids, consisting of a single stereoisomer, may exhibit secondary structures such as the α-helix or β-sheet. See α-Aminoacid-N-Caroboxy-Anhydrides and Related Heterocycles, H. R. Kricheldorf, Springer-Verlag, 1987. For example, poly(L-benzyl glutatmate) typically exhibits an α-helical conformation; however this secondary structure can be disrupted by a change of solvent or temperature (see Advances in Protein Chemistry XVI, P. Urnes and P. Doty, Academic Press, New York 1961). The secondary structure can also be disrupted by the incorporation of structurally dissimilar amino acids such as β-sheet forming amino acids (e.g. proline) or through the incorporation of amino acids with dissimilar stereochemistry (e.g. mixture of D and L stereoisomers), which results in poly(amino acids) with a random coil conformation. See Sakai, R.; Ikeda; S.; Isemura, T. Bull Chem. Soc. Japan 1969, 42, 1332-1336, Paolillo, L.; Temussi, P. A.; Bradbury, E. M.; Crane-Robinson, C. Biopolymers 1972, 11, 2043-2052, and Cho, I.; Kim, J. B.; Jung, H. J. Polymer 2003, 44, 5497-5500.

As used herein, the term “tacticity” refers to the stereochemistry of the poly(amino acid). A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer. Polymer tacticity is described in more detail in “Principles of Polymerization”, 3rd Ed., G. Odian, John Wiley & Sons, New York: 1991, the entire contents of which are hereby incorporated by reference.

As used herein, the phrase “unnatural amino acid side-chain group” refers to the side-chain group of amino acids not included in the list of 20 amino acids naturally occurring in proteins, as described above. Such amino acids include the D-isomer of any of the 20 naturally occurring amino acids. Unnatural amino acids also include homoserine, ornithine, norleucine, and thyroxine. Other unnatural amino acids side-chains are well known to one of ordinary skill in the art and include unnatural aliphatic side chains. Other unnatural amino acids include modified amino acids, including those that are N-alkylated, cyclized, phosphorylated, acetylated, amidated, azidylated, labelled, and the like. In some embodiments, an unnatural amino acid is a D-isomer. In some embodiments, an unnatural amino acid is a L-isomer.

As used herein, the phrase “amine-containing amino acid side-chain group” refers to natural or unnatural amino acid side-chain groups, as defined above, which comprise an amine moiety. The amine moiety may be primary, secondary, tertiary, or quaternary, and may be part of an optionally substituted group aliphatic or optionally substituted aryl group.

As used herein, the phrase N to P (N/P or N:P) refers to the ratio of protonatable nitrogens (N) to negatively charged phosphate groups in the DNA or RNA backbone (P).

As used herein, the phrase “living polymer chain-end” refers to the terminus resulting from a polymerization reaction that maintains the ability to react further with additional monomer or with a polymerization terminator.

As used herein, the term “termination” refers to attaching a terminal group to a polymer chain-end by the reaction of a living polymer with an appropriate compound. Alternatively, the term “termination” may refer to attaching a terminal group to an amine or hydroxyl end, or derivative thereof, of the polymer chain.

As used herein, the term “polymerization terminator” is used interchangeably with the term “polymerization terminating agent” and refers to a compound that reacts with a living polymer chain-end to afford a polymer with a terminal group. Alternatively, the term “polymerization terminator” may refer to a compound that reacts with an amine or hydroxyl end, or derivative thereof, of the polymer chain, to afford a polymer with a terminal group.

As used herein, the term “polymerization initiator” refers to a compound, which reacts with, or whose anion or free base form reacts with, the desired monomer in a manner that results in polymerization of that monomer. In certain embodiments, the polymerization initiator is the compound that reacts with an alkylene oxide to afford a polyalkylene oxide block. In other embodiments, the polymerization initiator is the amine salt described herein.

The term “aliphatic” or “aliphatic group,” as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon. This includes any oxidized form of nitrogen, sulfur, phosphorus, or silicon; the quaternized form of any basic nitrogen, or; a substitutable nitrogen of a heterocyclic ring including ═N— as in 3,4-dihydro-2H-pyrrolyl, —NH— as in pyrrolidinyl, or ═N(R^{†})— as in N-substituted pyrrolidinyl.

The term “unsaturated,” as used herein, means that a moiety has one or more units of unsaturation.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein at least one ring in the system is aromatic and wherein each ring in the system contains three to seven ring members. The term “aryl” may be used interchangeably with the term “aryl ring.”

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)_{0 4}R^{∘}; —(CH₂)_{0 4}OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)_{0 4}O(CH₂)_{0 1} Ph which may be substituted with R^{∘}; —CH═CHPh, which may be substituted with R^{∘}; —NO₂; —CN; —N₃; —(CH₂)_{0 4}N(R^{∘})₂; —(CH₂)_{0 4}N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, —(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. A suitable tetravalent substituent that is bound to vicinal substitutable methylene carbons of an “optionally substituted” group is the dicobalt hexacarbonyl cluster represented by

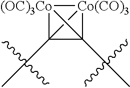

when depicted with the methylenes which bear it.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate(trimethylacetate), crotonate, 4-methoxy-crotonate, benzoate, p-benylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl)acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, in neutron scattering experiments, as analytical tools, or probes in biological assays.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g Fe₃O₄ and Fe₂O₃) particles. Similarly, semiconducting nanoparticles (e.g. cadmium selenide, cadmium sulfide, cadmium telluride) are useful as fluorescent labels. Other metal nanoparticles (e.g colloidal gold) also serve as primary labels.

“Secondary” labels include moieties such as biotin, or protein antigens, that require the presence of a second compound to produce a detectable signal. For example, in the case of a biotin label, the second compound may include streptavidin-enzyme conjugates. In the case of an antigen label, the second compound may include an antibody-enzyme conjugate. Additionally, certain fluorescent groups can act as secondary labels by transferring energy to another compound or group in a process of nonradiative fluorescent resonance energy transfer (FRET), causing the second compound or group to then generate the signal that is detected.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label,” “fluorescent group,” “fluorescent compound,” “fluorescent dye,” and “fluorophore,” as used herein, refer to compounds or moieties that absorb light energy at a defined excitation wavelength and emit light energy at a different wavelength. Examples of fluorescent compounds include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate,” as used herein refers to any material or macromolecular complex to which a functionalized end-group of a block copolymer can be attached. Examples of commonly used substrates include, but are not limited to, glass surfaces, silica surfaces, plastic surfaces, metal surfaces, surfaces containing a metallic or chemical coating, membranes (e.g., nylon, polysulfone, silica), micro-beads (eg., latex, polystyrene, or other polymer), porous polymer matrices (e.g., polyacrylamide gel, polysaccharide, polymethacrylate), macromolecular complexes (e.g., protein, polysaccharide).

The term “fusogenic peptide” refers to a peptide sequence that promotes escape from endolysomal compartments. Great efforts have been undertaken to further enhance endolysosomal escape and thus prevent lysosomal degradation. A key strategy has been adapted from viral elements that promote escape from the harsh endolysosomal environment and deliver their genetic information intact into the nucleus. Apart from complete virus capsids and purified capsid proteins, short amino acid sequences derived from the N-terminus of Haemophilus Influenza Haemagglutinin-2 have also been shown to induce pH-sensitive membrane disruption, leading to improved transfection of DNA-polycation polymer complexes in vitro. One such example is the INF7 peptide (GLFGAIAGFIENGWEGMIDGGGC). At neutral pH (pH 7.0) the INF peptide forms a random coil structure without fusogenic activity. However, this peptide undergoes a conformational change into an amphipathic α-helix at pH 5.0 and aggregates resulting in the formation of pores that destabilize endosomal membranes causing vesicle leakage. Indeed, the INF7 peptide has been used in combination with polymer based delivery systems and shown to tremendously enhance gene transfection activity without affecting cell cytotoxicity. Other synthetic fusogenic peptides may be used to aid endosome escape of our polymers, such as GALA (WEAALAEALAEALAEHLAEALAEALEALAA) and KALA (WEAKLAKALAKALAKHLAKALAKALKACEA) peptides. These peptides have been shown to successfully promote extensive membrane destabilization and subsequently, contribute to transfection enhancement.

As used herein, the term “targeting group” refers to any molecule, macromolecule, or biomacromolecule that selectively binds to receptors that are expressed or over-expressed on specific cell types. Targeting groups are well known in the art and include those described in International application publication number WO 2008/134731, published Nov. 6, 2008, the entirety of which is hereby incorporated by reference. In some embodiments, the targeting group is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide.

The term “oligopeptide”, as used herein refers to any peptide of 2-65 amino acid residues in length. In some embodiments, oligopeptides comprise amino acids with natural amino acid side-chain groups. In some embodiments, oligopeptides comprise amino acids with unnatural amino acid side-chain groups. In certain embodiments, oligopeptides are 2-50 amino acid residues in length. In certain embodiments, oligopeptides are 2-40 amino acid residues in length. In some embodiments, oligopeptides are cyclized variations of the linear sequences. In other embodiments, oligopeptides are 3-15 amino acid residues in length.

As used herein, the term “electron withdrawing group” refers to a group characterized by a tendency to attract electrons. Exemplary such groups are known in the art and include, by way of nonlimiting example, halogen, nitrile, nitro, carbonyl, and conjugated carbonyl.

### 3. Description of Exemplary Embodiments:

A. Cationic Polymers

As described generally above, one embodiment of the present invention provides a cationic polymer comprising a poly(amino acid) block. In certain embodiments, the cationic polymer may be comprised of a mixed poly(amino acid) block. In one embodiment, the cationic polymer is comprised of a poly(amino acid) block where all the amino acid units are in the L-configuration. In other embodiments, the cationic polymer is comprised of a poly(amino acid) block where the amino acid units are a mixture of D and L configurations.

In certain embodiments, the cationic polymer described above contains a mixture of primary and secondary amine groups on the side chain of the poly(amino acid). One of ordinary skill in the art will recognize that primary amine groups interact with phosphates in the polynucleotide to form the polyplex, whereas secondary amine groups function as a buffering group, or proton sponge, which aids in endosomal escape via endosome disruption. Ideally, one would select the optimum number of primary and secondary amines to both complex the polynucleotide and allow for sufficient endosomal escape, while limiting cytotoxicity.

In certain embodiments, the present invention provides a cationic polymer of formula I, or a salt thereof:

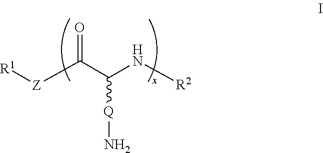

wherein:
   x is 10-250;
   each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   Z is a valence bond or a bivalent, saturated or unsaturated, straight or branched C₁₋₁₂ hydrocarbon chain, wherein 0-6 methylene units are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—;
   R¹ is hydrogen, —N₃, —CN, a suitable amine protecting group, a protected aldehyde, a protected hydroxyl, a suitable hydroxyl protecting group, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
   R² is selected from hydrogen, an optionally substituted aliphatic group, an acyl group, a sulfonyl group, or a fusogenic peptide.

In certain embodiments, the x group is about 10 to about 250. In certain embodiments, the x group is about 25. In other embodiments x is about 10 to about 50. In other embodiments, x is about 50. According to yet another embodiment, x is about 75. In other embodiments, x is about 100. In certain embodiments, x is about 40 to about 80. In other embodiments, x is selected from 10±5, 15±5, 25±5, 50±5, 75±10, 100±10, or 125±10.

In certain embodiments, Z is a —NH— group. In certain embodiments, Z is a valence bond. In some embodiments, Z is a bivalent, saturated or unsaturated, straight or branched C₁₋₈ hydrocarbon chain, wherein 0-3 methylene units are independently replaced by —O—, —NH—, —S—, —OC(O)—, —C(O)O—, —C(O)—, —NHC(O)—, or —C(O)NH—.

In some embodiments, R¹ is an optionally substituted aliphatic group. In certain embodiments, the R¹ group is a saturated or unsaturated C₁₋₁₂ alkyl chain. In other embodiments, the R¹ group is a pentyl group. In other embodiments, the R¹ group is a hexyl group. In other embodiments, the R¹ group is a hydrogen atom. In other embodiments, the R¹ group is a quaternized triethylamine group. In some embodiments, where Z comprises an amine, R¹ is a suitable amine protecting group. In some embodiments, where Z comprises a hydroxyl, R¹ is a suitable hydroxyl protecting group. In some embodiments, R¹ is or comprises an azide group. In some embodiments, R¹ is or comprises an alkynyl group.

In certain embodiments, the R² group is an acetyl group. In some embodiments, the R² group is a hydrogen atom. In some embodiments, R² is acyl. In some embodiments, R² is a fusogenic peptide.

In certain embodiments, the Q group is a chemical moiety representing an oligomer of ethylene amine, —(NH₂—CH₂—CH₂)—. In some embodimnets, Q is a bivalent, saturated or unsaturated, straight or branched C₁₋₂₀ alkylene chain, wherein 0-9 methylene units of Q are independently replaced by —NH—, —C(O)—, —NHC(O)—, or —C(O)NH—. In certain embodiments, Q is a branched alkylene chain wherein one or more methine carbons is replaced with a nitrogen atom to form a trivalent amine group. Specific examples of Q groups can be found in Table 1a, Table 1b, and Table 1c.

TABLE 1a

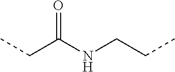

i

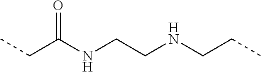

ii

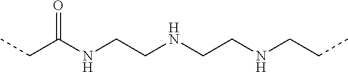

iii

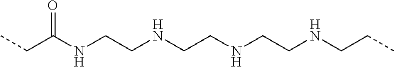

iv

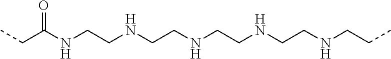

v

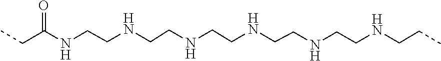

vi

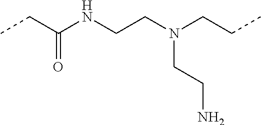

vii

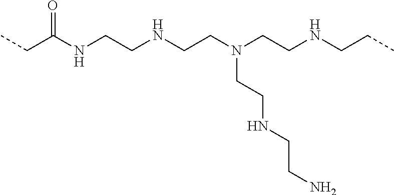

viii

TABLE 1b

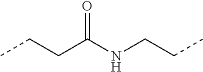

ix

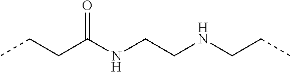

x

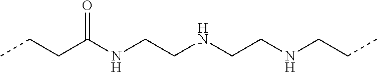

xi

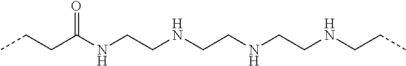

xii

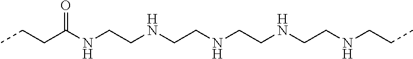

xiii

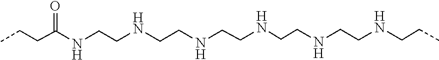

xiv

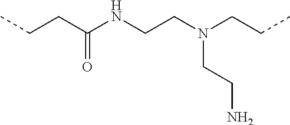

xv

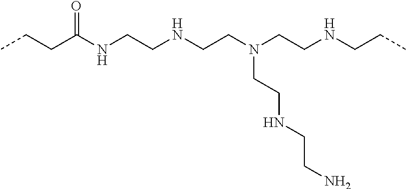

xvi

TABLE 1c

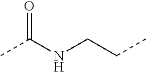

xvii

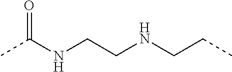

xviii

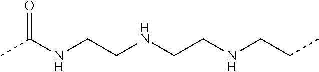

xix

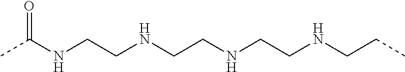

xx

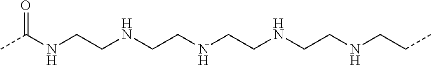

xxi

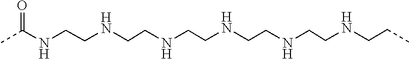

xxii

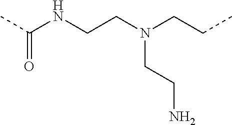

xxiii

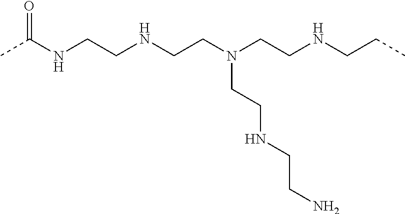

xxiv

One skilled in the art will recognize that the stereochemistry of the poly(amino acid) represented in Formula I is undefined. It is understood that this depiction can represent an all L conformation, an all D conformation, or any random mixture of D and L isomers.

Exemplary polymers, or salts thereof, of Formula I are set forth in Table 2, wherein x is 10-250 and y is 10-250.

TABLE 2

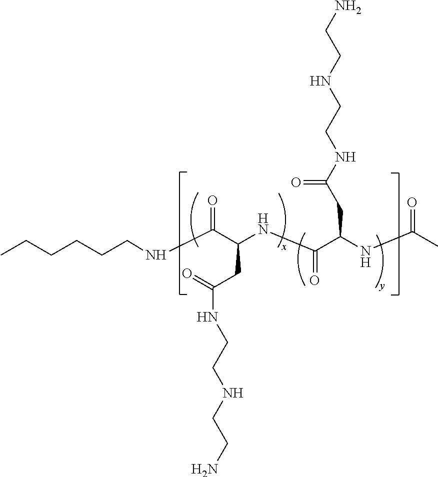

i

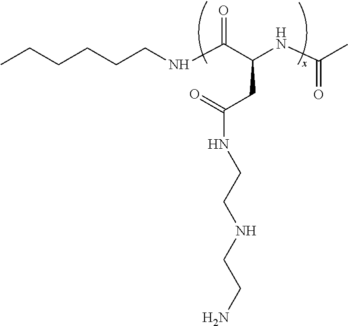

j

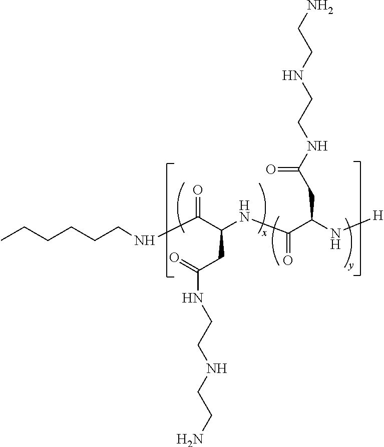

k

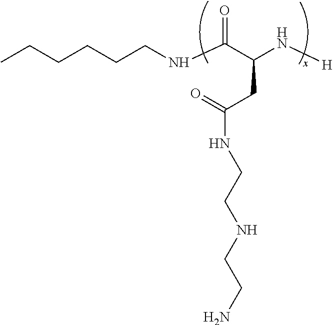

l

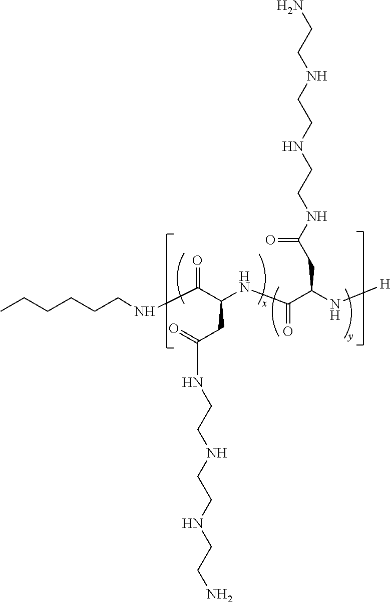

m

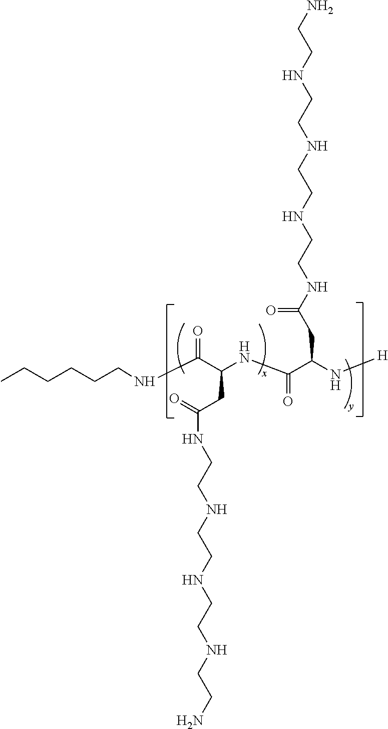

n

In certain embodiments, the present invention provides a copolymer of formula II:

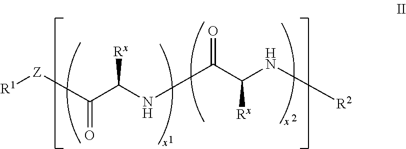

wherein each of R¹, Q, Z, and R² is as defined above and as described in classes and subclasses herein, both singly and in combination;
   x¹ is 0 to 250;
   x² is 0 to 250, provided that x¹ and x² are not simultaneously zero such that the sum of x¹ and x² is greater than or equal to 5; and
   each R^{x} group is independently selected from an amino acid side-chain group selected from benzyl aspartate, benzyl glutamate, t-butyl aspartate, t-butyl glutamate, methyl aspartate, methyl glutamate, alkyl aspartate or alkyl glutamate;

In certain embodiments, x¹ is about 10 to about 250. In certain embodiments, x¹ is about 25. In certain embodiments, x¹ is about 10. In certain embodiments, x¹ is about 15. In certain embodiments, x¹ is about 20. In other embodiments x¹ is about 10 to about 50. In other embodiments, x¹ is about 50. According to yet another embodiment, x¹ is about 75. In other embodiments, x¹ is about 100. In other embodiments, x¹ is selected from 10±5, 15±5, 25±5, 50±5, 75±10, 100±10, or 125±10.

In certain embodiments, the x² group is about 10 to about 250. In certain embodiments, the x² group is about 25. In certain embodiments, the x² group is about 10. In certain embodiments, the x² group is about 15. In certain embodiments, the x² group is about 20. In other embodiments x² is about 10 to about 50. In other embodiments, x² is about 50. According to yet another embodiment, x² is about 75. In other embodiments, x² is about 100. In other embodiments, x² is selected from 10±5, 15±5, 25±5, 50±5, 75±5, 100±10, or 125±10.

As is readily apparent, the R^{x} group is a natural or unnatural amino acid side-chain group comprising an ester moiety capable of undergoing aminolysis. One of ordinary skill in the art would recognize that many readily available amine-containing compounds are suitable for such aminolysis reactions. Exemplary amine derivatives suitable for such aminolysis are set forth in Table 3, below.

TABLE 3

| | |
|---|---|
| 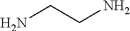 | 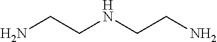 |

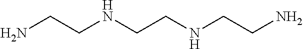

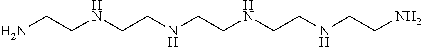

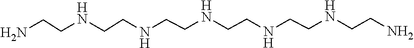

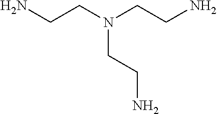

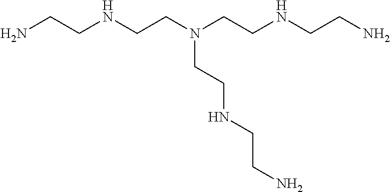

When a compound of Formula II is treated with a suitable amine under aminolysis conditions, a rearrangement to a beta-amino acid or racemization of the side chain's stereocenter is a possible side reaction. The mechanism for this rearrangment is detailed in Kataoka et. al. Reactive and Functional Polymers, 2007, 67, 1361-1372 and is represented in Scheme 1, below.

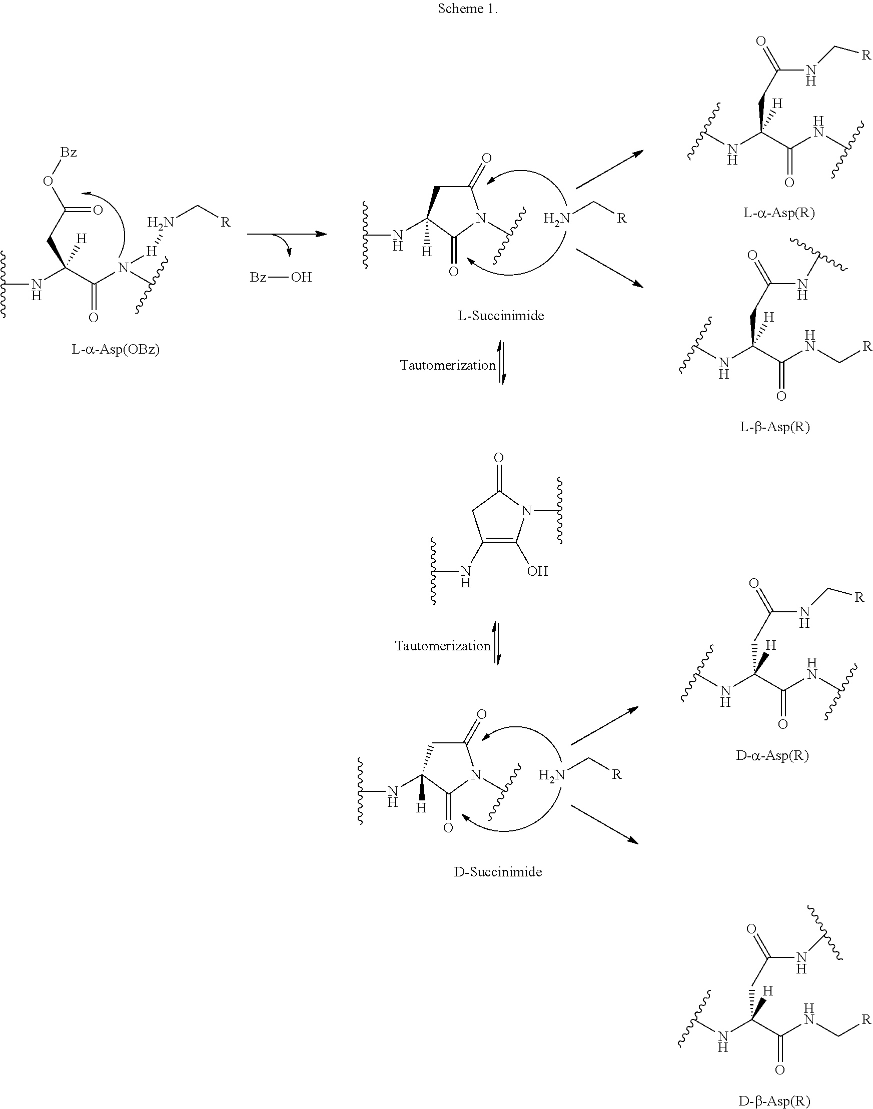

The exact reaction conditions (e.g. temperature, solvent polarity, equivalents of amine) all influence the nature of the side reactions that can occur. Thus, during the course of aminolysis, one can envision four classes of product compounds: a case where both racemization of the stereocenter and rearrangement to the beta amino acid occurs, a case where only racemization occurs, a case where only rearrangement to the beta amino acid occurs, and a case where neither racemization nor rearrangement occurs. Without wishing to be bound to any particular theory, it is believed that if the starting material is enriched in either L or D stereocenters, then the resulting product will retain at least a portion of, and, in some embodiments, the majority of, the original stereochemical enrichment. One of ordinary skill in the art will recognize that such partial racemization and/or rearrangement, when present, results in the formation of a mixed block.

In certain embodiments, the present invention provides a copolymer of formula I-a, or a salt thereof:

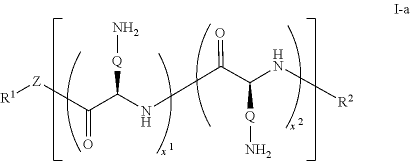

wherein each of R¹, Q, Z, x¹, x² and R² is as defined above and as described in classes and subclasses herein, both singly and in combination.

In certain embodiments, the present invention provides a copolymer of formula I-b, or a salt thereof:

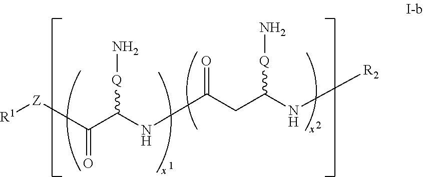

wherein each of R¹, Q, Z, x¹, x² and R² is as defined above and as described in classes and subclasses herein, both singly and in combination.

It will be appreciated by one skilled in the art that each of formulae I, I-a, and I-b represent a polyamine, or a salt thereof When any of formulae I, I-a, and I-b is dissolved in an aqueous solution at pH 4-9, it will be appreciated that a plurality of the amino groups will exist as an ammonium salt (—NH₃⁺) with a suitable anion, while other amino groups will exist as the free base (—NH₂). One skilled in the art will readily recognize that the ratio between the protonated ammonium salt and the free base is heavily influenced by pH, as lower pH values will result in a high population of the ammonium salt and high pH values will result in a high population of the free base. Thus, it is contemplated that the polyamines of formulae I, I-a, and I-b exist as a polycation in aqueous solution.

As generally described above, a suitable anion describes any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

B. Polynucleotide Encapsulation

The present invention provides the preparation of a polyplex formed by the addition of a cationic polymer and a polynucleotide.

In water, such cationic copolymers co-assemble with polynucleotides through electrostatic interactions between the cationic side chains of the polymer and the anionic phosphates of the polynucleotide to form a polyplex. In some cases, the number of phosphates on the polynucleotides may exceed the number of cationic charges on the multiblock copolymer. It will be appreciated that multiple polymers may be used to achieve charge neutrality (i.e. N/P=1) between the polymer and encapsulated polynucleotide. It will also be appreciated that when an excess of polymer is used to encapsulate a polynucleotide, the polymer/polynucleotide complex can possess an overall positive charge (i.e. N/P>1).

As described herein, polyplexes of the present invention can be prepared with any polynucleotide agent. In one embodiment, the encapsulated polynucleotide is a plasmid DNA (pDNA). As used herein, pDNA is defined as a circular, double-stranded DNA that contains a DNA sequence (cDNA or complementary DNA) that is to be expressed in cells either in culture or in vivo. The size of pDNA can range from 3 kilo base pairs (kb) to greater than 50 kb. The cDNA that is contained within plasmid DNA is usually between 1-5 kb in length, but may be greater if larger genes are incorporated. pDNA may also incorporate other sequences that allow it to be properly and efficiently expressed in mammalian cells, as well as replicated in bacterial cells. In certain embodiments, the encapsulated pDNA expresses a therapeutic gene in cell culture, animals, or humans that possess a defective or missing gene that is responsible for and/or correlated with disease.

In certain embodiments, an encapsulated polynucleotide is capable of silencing gene expression via RNA interference (RNAi). As defined herein, RNAi is a cellular mechanism that suppresses gene expression during translation and/or hinders the transcription of genes through destruction of messenger RNA (mRNA). Without wishing to be bound by any particular theory, it is believed that endogenous double-stranded RNA located in the cell are processed into 20-25 nt short-interfering RNA (siRNA) by the enzyme Dicer. siRNA subsequently binds to the RISC complex (RNA-induced silencing nuclease complex), and the guide strand of the siRNA anneals to the target mRNA. The nuclease activity of the RISC complex then cleaves the mRNA, which is subsequently degraded (Nat. Rev. Mol. Cell Biol., 2007, 8, 23).

In one embodiment, an encapsulated polynucleotide is a siRNA. As used herein, siRNA is defined as a linear, double-stranded RNA that is 20-25 nucleotides (nt) in length and possesses a 2 nt, 3′ overhang on each end which can induce gene knockdown in cell culture or in vivo via RNAi. In certain embodiments, the encapsulated siRNA suppresses disease-relevant gene expression in cell culture, animals, or humans.

In certain embodiments, the encapsulated polynucleotide is pDNA that expresses a short-hairpin RNA (shRNA). As used herein, shRNA is a linear, double-stranded RNA, possessing a tight hairpin turn, which is synthesized in cells through transfection and expression of a exogenous pDNA. Without wishing to be bound by any particular theory, it is believed that the shRNA hairpin structure is cleaved to produce siRNA, which mediates gene silencing via RNA interference. In certain embodiments, the encapsulated shRNA suppresses gene expression in cell culture, animals, or humans that are responsible for a disease via RNAi.

In certain embodiments, the encapsulated polynucleotide is a microRNA (miRNA). As used herein, miRNA is a linear, single-stranded RNA that ranges between 21-23 nt in length and regulates gene expression via RNAi (Cell, 2004, 116, 281). In certain embodiments, an encapsulated miRNA suppresses gene expression in cell culture, animals, or humans that are responsible for a disease via RNAi.

In another embodiment, an encapsulated polynucleotide is a messenger RNA (mRNA). As used herein, mRNA is defined as a linear, single stranded RNA molecule, which is responsible for translation of genes (from DNA) into proteins. In certain embodiments, the encapsulated mRNA is encoded from a plasmid cDNA to serve as the template for protein translation. In certain embodiments, an encapsulated mRNA translates therapeutic proteins, in vitro and/or in vivo, which can treat disease.

In certain embodiments, an encapsulated polynucleotide is an antisense RNA (asRNA). As used herein, asRNA is a linear, single-stranded RNA that is complementary to a targeted mRNA located in a cell. Without wishing to be bound by any particular theory, it is believed that asRNA inhibits translation of a complementary mRNA by pairing with it and obstructing the cellular translation machinery. It is believed that the mechanism of action for asRNA is different from RNAi because the paired mRNA is not destroyed. In certain embodiments, an encapsulated asRNA suppresses gene expression in cell culture, animals, or humans that are responsible for a disease by binding mRNA and physically obstructing translation.

In certain embodiments, the N/P ratio will be greater than 1. In certain embodiments, the N/P ratio will be range 2 to 50. In some embodiments, the N/P ratio will be selected from 2, 3, or 4. In certain embodiments, the N/P ratio is 5. In yet other embodiments, the N/P ratio is 10. In some embodiments, the N/P ratio is selected from 15, 20, 25, 30, 35, 40, or 50. In other embodiments, the N/P ratio is from 5 to 10. In certain embodiments, the N/P ratio is about 5 or about 10. In yet other embodiments, the N/P ratio is from 4 to 15.

In certain embodiments, the present invention provides a polyplex having a polynucleotide encapsulated therein, comprising a cationic polymer of formula I or a salt thereof:

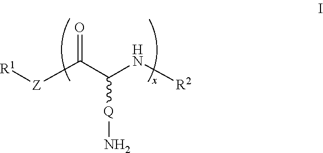

wherein each of R¹, Q, Z, x, and R² is as defined above and as described in classes and subclasses herein, both singly and in combination.

In some embodiments, the present invention provides a polyplex having a polynucleotide encapsulated therein, comprising a cationic polymer of formula I, or a salt thereof, wherein each variable is as defined and described herein, both singly and in combination.

In certain embodiments, the present invention provides a polyplex having a polynucleotide encapsulated therein, comprising a cationic polymer of formula I-a or a salt thereof:

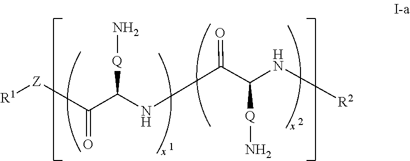

wherein each of R¹, Q, Z, x¹, x² and R² is as defined above and as described in classes and subclasses herein, both singly and in combination.

In some embodiments, the present invention provides a polyplex having a polynucleotide encapsulated therein, comprising a cationic polymer of formula I-a, or a salt thereof, wherein each variable is as defined and described herein, both singly and in combination.

In certain embodiments, the present invention provides a polyplex having a polynucleotide encapsulated therein, comprising a cationic polymer of formula I-b:

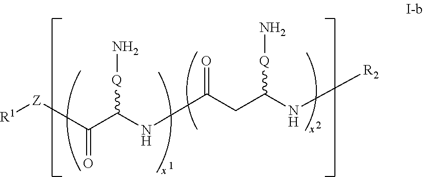

wherein each of R¹, Q, Z, x¹, x² and R² is as defined above and as described in classes and subclasses herein, both singly and in combination.

In some embodiments, the present invention provides a polyplex having a polynucleotide encapsulated therein, comprising a cationic polymer of formula I-b, or a salt thereof, wherein each variable is as defined and described herein, both singly and in combination.

In certain embodiments, the polynucleotide complexation is performed at neutral pH. In other embodiments, the polynucleotide complexation is performed at pH of 4-8. In other embodiments, the polynucleotide complexation is performed at pH of about 7.4. In other embodiments, the polynucleotide complexation is performed at pH of 6.5-7.5.

In some embodiments, the present invention provides a composition comprising a compound of formula I and at least one compound selected from a compound of formula I-a and/or I-b, wherein each variable is as defined and described herein, both singly and in combination.

In certain embodiments, the present invention provides a polyplex having a polynucleotide encapsulated therein, wherein the polyplex comprises a compound of formula I and at least one compound selected from a compound of formula I-a and/or I-b, wherein each variable is as defined and described herein, both singly and in combination.

C. Polyplex PEGylation

The present invention further provides the preparation of a polyplex formed by the addition of a cationic polymer and a polynucleotide, followed by the covalent attachment of PEG to the polyplex to form a PEG-conjugated polyplex.

One of ordinary skill in the art will recognize that multiple avenues exist to conjugate the PEG onto the polyplex. Generally, excess amines present within the polyplex will react with suitable electrophiles to form covalent bonds. Suitable electrophiles include, but are not limited to, maleimides, activated esters, esters, and aldehydes. It is also important to recognize that the pH of the solution will affect the reactivity of the excess amines present within the polyplex. At low pH, the amines will predominately exist as an ammonium salt, and the reaction rate of the ammonium salt with the electrophile will be very low. However, as the pH approaches basic conditions (>7), the amines will have a higher percentage of free amine compared to ammonium salts. When the percentage of free amines increases, the reaction rate with a suitable electrophile will also increase. Thus, it is advantageous to select a pH that allows for the highest reaction rate (basic pH) without causing an adverse effect to the polynucleotide. In some embodiments, the pH of the PEGylation reaction solution is 4.0-9.0. In some embodiments, the pH of the PEGylation reaction solution is 5.0-6.0. In other embodiments, the pH of the PEGylation reaction solution is 6.0-7.0. In some embodiments, the pH of the PEGylation reaction solution is 7.0-8.0. In yet other embodiments, the pH of the PEGylation reaction solution is about 7.0. In another embodiment, the pH of the PEGylation reaction solution is about 7.5. In yet another embodiments, the pH of the PEGylation reaction solution is about 7.4.

In certain embodiments, the present invention provides a cationic polymer of formula III or a salt thereof:

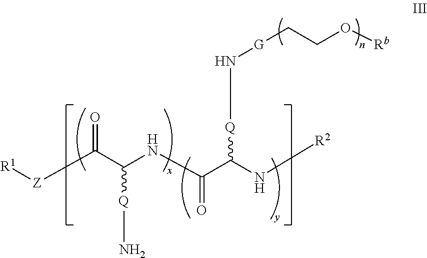

wherein each of R¹, Q, Z, x, and R² is as defined above and as described in classes and subclasses herein, both singly and in combination;
   y is 1-200;
   n is 40-500;
   each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   each R^{b} is independently —CH₃, a saturated or unsaturated alkyl moiety, an alkyne containing moiety, an azide containing moiety, a protected amine moiety, an aldehyde or protected aldehydes containing moiety, a thiol or protected thiol containing moiety, a cyclooctyne containing moiety, difluorocyclooctyne containing moiety, a nitrile oxide containing moiety, an oxanorbornadiene containing moiety, or an alcohol or protected alcohol containing moiety.

In certain embodiments, y is about 1 to about 200. In certain embodiments, y is about 25. In certain embodiments, y is about 10. In certain embodiments, y is about 20. In certain embodiments, y is about 15. In other embodiments y is about 1 to about 25. In other embodiments, y is about 50. According to yet another embodiment, y is about 25-75. In other embodiments, y is about 100. In other embodiments, y is selected from 10±5, 15±5, 25±5, 50±5, 75±10, 100±10, or 125±10.

As defined generally above, n is 40-500. In certain embodiments n is about 225. In some embodiments, n is about 275. In other embodiments, n is about 110. In other embodiments, n is about 40 to about 60. In other embodiments, n is about 60 to about 90. In still other embodiments, n is about 90 to about 150. In other embodiments, n is about 150 to about 200. In some embodiments, n is about 200 to about 300, about 300 to about 400, about 400 to about 500. In still other embodiments, n is about 250 to about 280. In other embodiments, n is about 300 to about 375. In other embodiments, n is about 400 to about 500. In certain embodiments, n is selected from 50±10. In other embodiments, n is selected from 80±10, 115±10, 180±10, 225±10, 275±10, or 450±10.

In some embodiments, R^{b} is an optionally substituted aliphatic group containing an alkyne. In some embodiments, R^{b} is an optionally substituted aliphatic group containing an azide. In some embodiments, R^{b} is an optionally substituted aliphatic group containing an aldehyde or protected aldehyde. In some embodiments, R^{b} is an optionally substituted aliphatic group containing a thiol or protected thiol. In some embodiments, R^{b} is an optionally substituted aliphatic group containing a cyclooctyne group. In some embodiments, R^{b} is an optionally substituted aliphatic group containing a difluorocyclooctyne group. In some embodiments, R^{b} is an optionally substituted aliphatic group containing a oxanobornadiene group. In certain embodiments, R^{b} is —CH₂CH₂N₃. In other embodiments, R^{b} is —CH₃. In some embodiments, a polymer chain comprises a mixture of —CH₂CH₂N₃ and —CH₃ groups at the R^{b} position.

In certain embodiments, the G group is a valence bond. In other embodiments, the G group comprises a carbonyl group. In other embodiments, the G group is represented by a moiety in Table 4.

TABLE 4

| | |
|---|---|
| 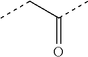 | 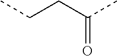 |
| o | p |
| 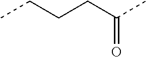 | 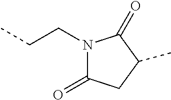 |
| q | r |

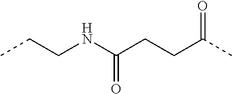

s

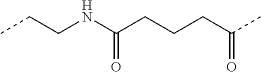

t

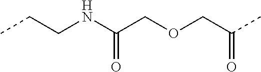

u

In some embodiments, the present invention provides a cationic polymer of formula III, or a salt thereof, wherein each variable is as defined and described herein, both singly and in combination.

In some embodiments, the present invention provides a PEG-conjugated polyplex having a polynucleotide encapsulated therein, comprising a compound of formula III.

In some embodiments, the present invention provides a polyplex having a polynucleotide encapsulated therein, comprising a cationic polymer of formula III, or a salt thereof, wherein each variable is as defined and described herein, both singly and in combination.

Exemplary polymers, or salts thereof, of Formula III are set forth in Table 5, wherein x is 10-250 and y is 10-250.

TABLE 5

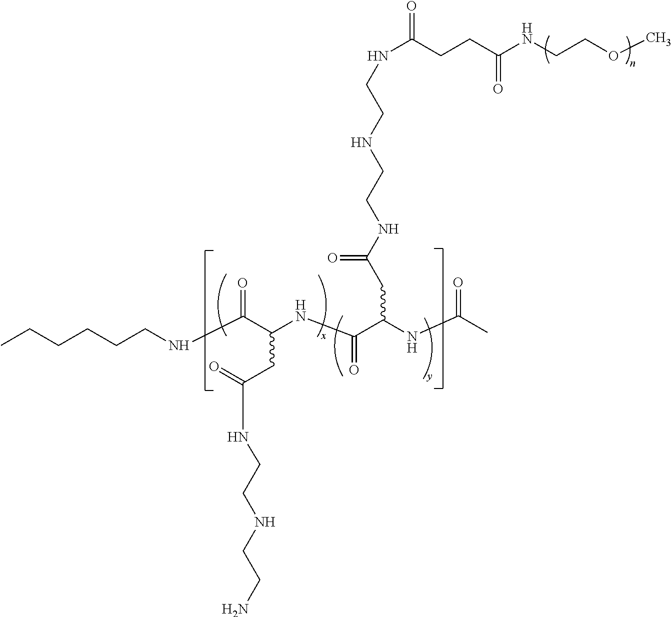

v

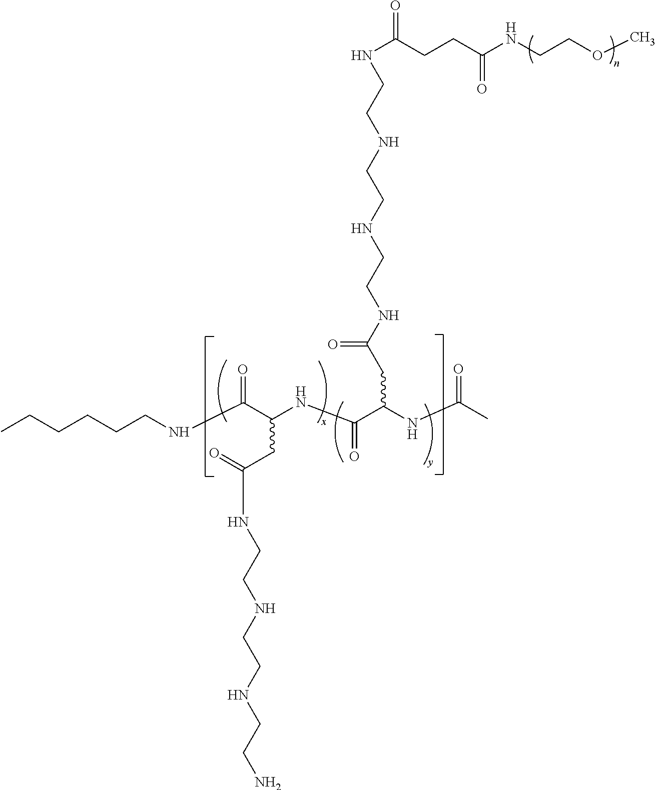

w

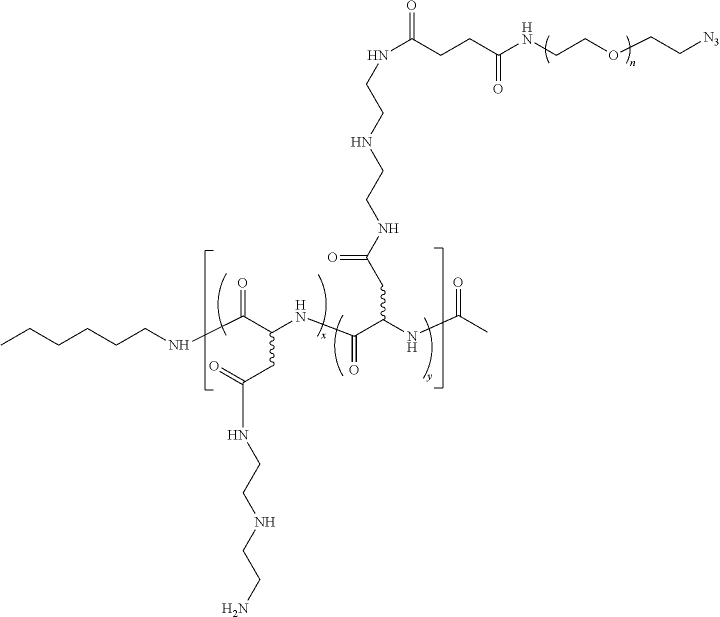

x

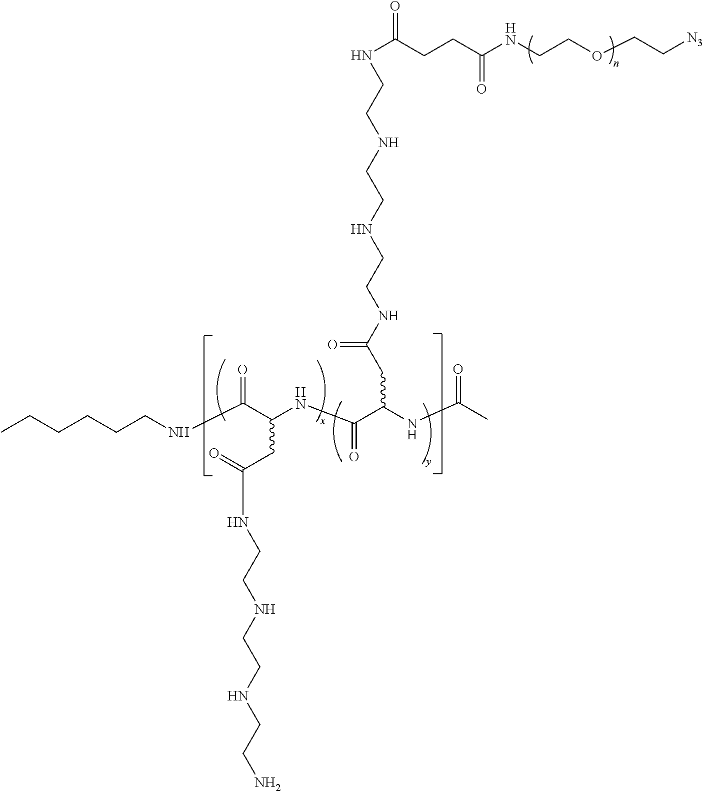

y

In certain embodiments, the present invention provides method of preparation for a PEG-conjugated polyplex having a polynucleotide encapsulated therein, comprising a cationic polymer of formula III or a salt thereof:

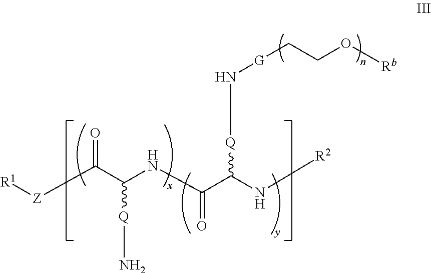

wherein each of R¹, Q, Z, G, x, y, n, R^{b} and R² is as defined above and as described in classes and subclasses herein, both singly and in combination;
   comprising the steps of:
(1) providing a polyplex having a polynucleotide encapsulated therein, comprising a cationic polymer of formula I, as defined above and described in classes and subclasses herein;
(2) optionally adjusting the pH of the polyplex solution to pH 4.0-9.0; and
(3) conjugating a compound of formula IV:

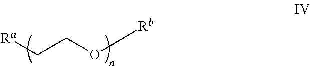

wherein each of R^{b} and n is as defined above and as described in classes and subclasses herein, both singly and in combination; and
R^{a} is or comprises a suitable electrophile;
to the polyplex by reaction of the electrophile of formula IV and an amine group of Formula I to afford the cationic polymer of formula III.

As generally described above, an electrophile of R^{a} is generally described as a moiety capable of reacting with a nucleophile to form a new covalent bond. In certain embodiments, a suitable electrophile is one that is capable of reacting with an amine derivative. Suitable electrophiles include, but are not limited to maleimide derivatives, activated ester moieties, esters, and aldehyde moieties.

It will be appreciated by one skilled in the art that the copolymer of formula III represents a random, mixed copolymer of free amines or ammonium salts and amines that have reacted with a compound of formula IV to provide a covalent bond attaching the grafted PEG chain to the poly(amino acid) backbone. Thus, a mixture of free amines or ammonium salts and PEG chains now represents the side chains of the poly(amino acid) copolymer. It will be appreciated that if and only if the x group of formula III is zero, then each and every amine would have reacted with a compound of formula IV and no free amine or ammoniums salts would exist in formula III.

Exemplary compounds of formula IV can be found in Table 6a and 6b, wherein each n is independently 40-500.

TABLE 6a

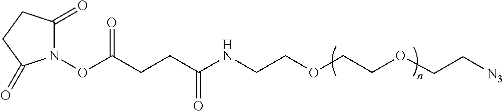

i

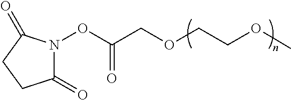

ii

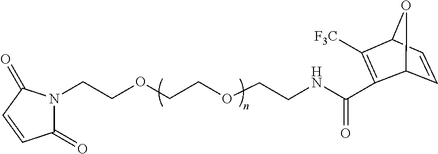

iii

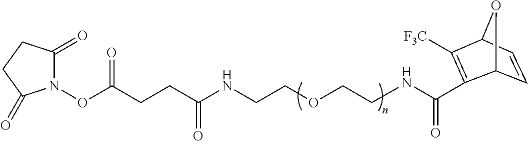

vi

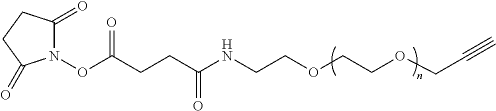

v

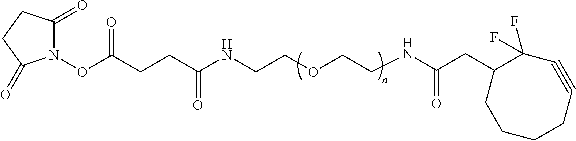

iv

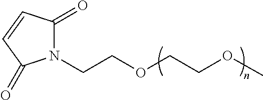

vii

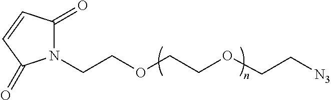

viii

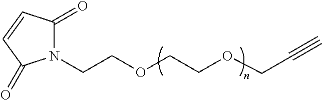

ix

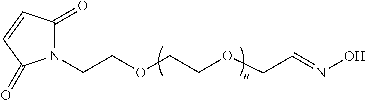

x

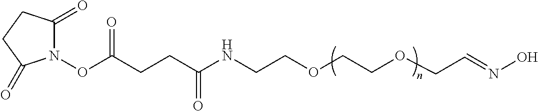

xi

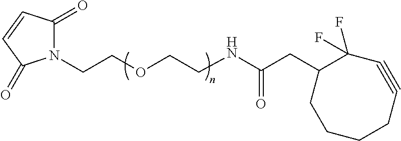

xii

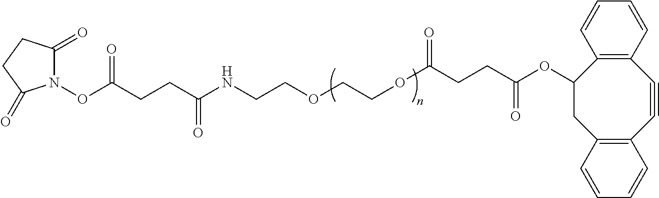

xiii

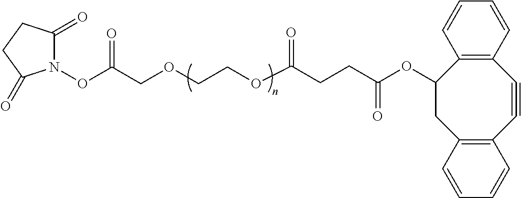

xiv

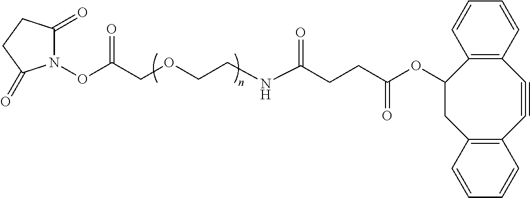

xv

TABLE 6b

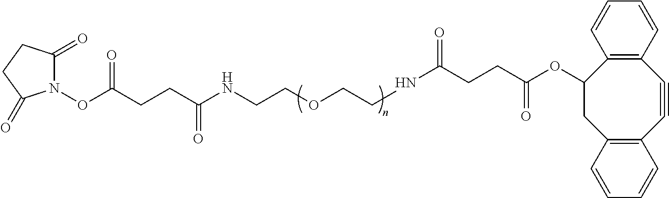

xvi

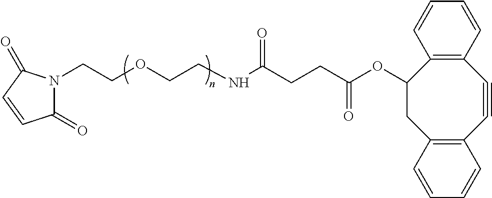

xvii

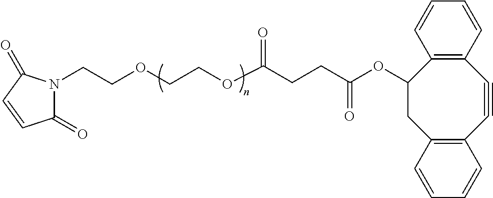

xviii

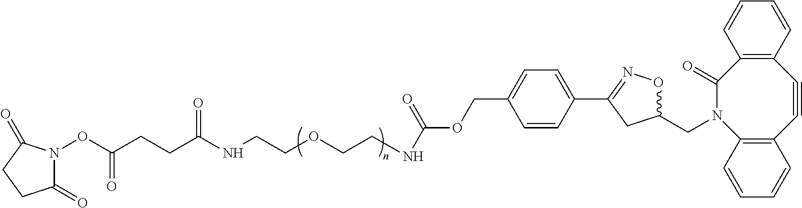

xix

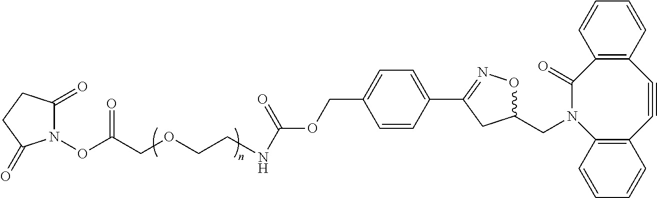

xx

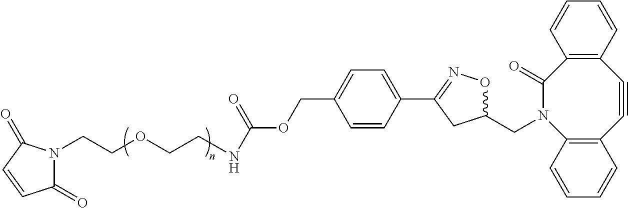

xxi

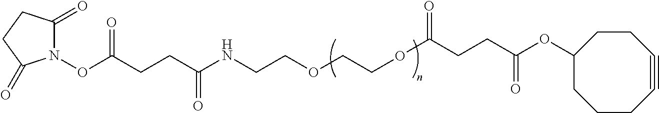

xxii

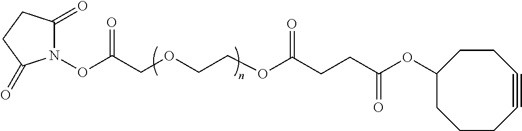

xxiii

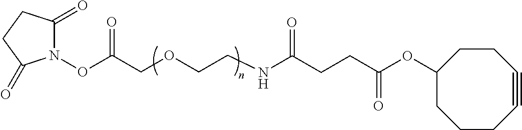

xxiv

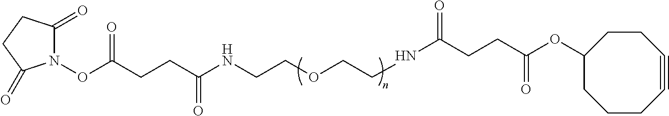

xxv

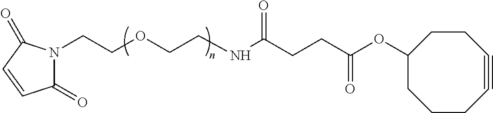

xxvi

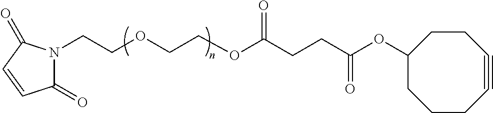

xxvii

In certain embodiments, the present invention provides a PEG-conjugated cationic polymer of formula III-a or a salt thereof:

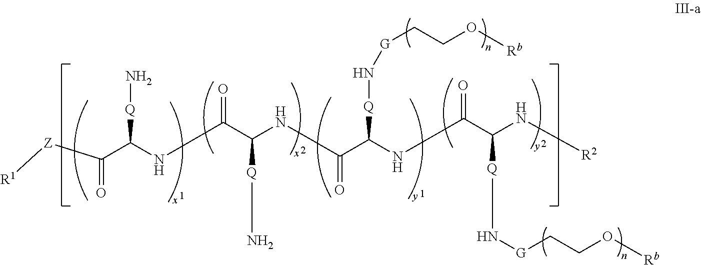

wherein each of R¹, Q, Z, G, x¹, x², n, R^{b} and R² is as defined above and as described in classes and subclasses herein, both singly and in combination;
   y¹ is 1-200;
   y² is 1-200.

In certain embodiments, y¹ is about 1 to about 200. In certain embodiments, y¹ is about 25. In other embodiments, y¹ is about 5. In certain embodiments, y¹ is about 10. In other embodiments, y¹ is about 15. In other embodiments, y¹ is about 20. In other embodiments y¹ is about 1 to about 25. In other embodiments, y¹ is about 50. According to yet another embodiment, y¹ is about 25-75. In other embodiments, y¹ is about 100. In other embodiments, y¹ is selected from 10±5, 15±5, 25±5, 50±5, 75±10, 100±10, or 125±10.

In certain embodiments, y² is about 1 to about 200. In certain embodiments, y² is about 25. In other embodiments, y² is about 5. In certain embodiments, y² is about 10. In other embodiments, y² is about 15. In other embodiments, y² is about 20. In other embodiments y² is about 1 to about 25. In other embodiments, y² is about 50. According to yet another embodiment, y² is about 25-75. In other embodiments, y² is about 100. In other embodiments, y² is selected from 10±5, 15±5, 25±5, 50±5, 75±10, 100±10, or 125±10.

In some embodiments, the present invention provides a cationic polymer of formula III-a, or a salt thereof, wherein each variable is as defined and described herein, both singly and in combination.

In some embodiments, the present invention provides a polyplex, having a polynucleotide encapsulated therein, comprising a cationic polymer of formula III-a.

In some embodiments, the present invention provides a polyplex having a polynucleotide encapsulated therein, comprising a cationic polymer of formula III-a, or a salt thereof, wherein each variable is as defined and described herein, both singly and in combination.

In certain embodiments, the present invention provides a method for preparing for a PEG-conjugated polyplex having a polynucleotide encapsulated therein, comprising a cationic polymer of formula III-a or a salt thereof:

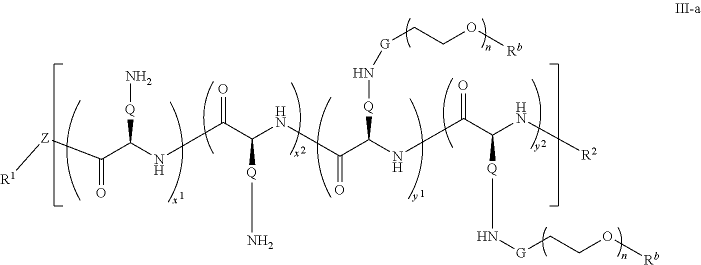

wherein each of R¹, Q, Z, G, x¹, x², y¹, y², n, R^{b} and R² is as defined above and as described in classes and subclasses herein, both singly and in combination;
   comprising the steps of:
(1) providing a polyplex having a polynucleotide encapsulated therein, comprising a cationic polymer of formula I-a, as defined above and described in classes and subclasses herein;
(2) optionally adjusting the pH of the polyplex solution to pH 4.0-9.0
(3) conjugating a compound of formula IV:

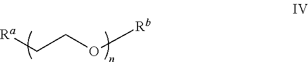

wherein each of R^{a}, R^{b} and n is as defined above and as described in classes and subclasses herein, both singly and in combination;
to the polyplex by reaction of an electrophile of formula IV and at least one amine group of formula I-a to afford a cationic polymer of formula III-a

In certain embodiments, the present invention provides a compound of formula III-b or a salt thereof:

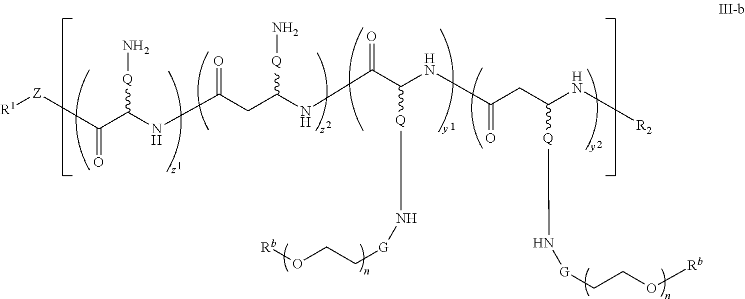

wherein each of R¹, Q, Z, G, y¹, y², n, R^{b} and R² is as defined above and as described in classes and subclasses herein, both singly and in combination;
   z¹ is 0-250;
   z² is 0-250.

In certain embodiments, z¹ is about 1 to about 200. In certain embodiments, z¹ is about 25. In other embodiments, z¹ is about 5. In certain embodiments, z¹ is about 10. In other embodiments, z¹ is about 15. In other embodiments, z¹ is about 20. In other embodiments z¹ is about 1 to about 25. In other embodiments, z¹ is about 50. According to yet another embodiment, z¹ is about 25-75. In other embodiments, z¹ is about 100. In other embodiments, z¹ is selected from 10±5, 15±5, 25±5, 50±5, 75±10, 100±10, or 125±10.

In certain embodiments, z² is about 1 to about 200. In certain embodiments, z² is about 25. In other embodiments, z² is about 5. In certain embodiments, z² is about 10. In other embodiments, z² is about 15. In other embodiments, z² is about 20. In other embodiments z² is about 1 to about 25. In other embodiments, z² is about 50. According to yet another embodiment, z² is about 25-75. In other embodiments, z² is about 100. In other embodiments, z² is selected from 10±5, 15±5, 25±5, 50±5, 75±10, 100±10, or 125±10.

In some embodiments, the present invention provides a cationic polymer of formula III-b, or a salt thereof, wherein each variable is as defined and described herein, both singly and in combination.

In some embodiments, the present invention provides a polyplex, having a polynucleotide encapsulated therein, comprising a cationic polymer of formula III-b.

In some embodiments, the present invention provides a polyplex having a polynucleotide encapsulated therein, comprising a cationic polymer of formula III-b, or a salt thereof, wherein each variable is as defined and described herein, both singly and in combination.

In certain embodiments, the present invention provides method of preparation for a PEG-conjugated polyplex having a polynucleotide encapsulated therein, comprising a cationic polymer of formula III-b or a salt thereof:

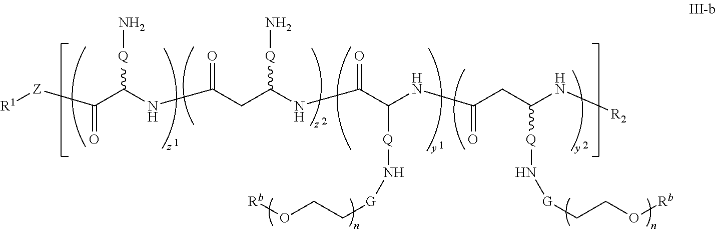

wherein each of R¹, Q, Z, G, z¹, z², y¹, y², n, R^{b} and R² is as defined above and as described in classes and subclasses herein, both singly and in combination;
   from a compound of formula IV:

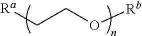

wherein each of R^{a}, R^{b} and n is as defined above and as described in classes and subclasses herein, both singly and in combination;
   comprising the steps of:
(1) providing a polyplex having a polynucleotide encapsulated therein, comprising a cationic polymer of formula I-b, as defined above and described in classes and subclasses herein;
(2) optionally adjusting the pH of the polyplex solution to pH 4.0-9.0; and
(3) conjugating the PEG to the polyplex by the reaction of an electrophile of Formula IV and an amine group of Formula I-b to afford a cationic polymer of Formula III-b.

In some embodiments, the present invention provides a composition comprising a compound of formula III and at least one compound selected from a compound of formula III-a and/or III-b.

In certain embodiments, the present invention provides a polyplex having a polynucleotide encapsulated therein, wherein the polyplex comprises a compound of formula III and at least one compound selected from a compound of formula III-a and/or III-b.

D. Targeting Group Attachment

PEG-conjugated polyplexes described herein can be modified to enable active cell-targeting to maximize the benefits of current and future therapeutic agents. Because these polyplexes typically possess diameters greater than 20 nm, they exhibit dramatically increased circulation time when compared to stand-alone drugs due to minimized renal clearance. This unique feature of nanovectors leads to selective accumulation in diseased tissue, especially cancerous tissue due to the enhanced permeation and retention effect (“EPR”). The EPR effect is a consequence of the disorganized nature of the tumor vasculature, which results in increased permeability of polymer therapeutics and drug retention at the tumor site. In addition to passive cell targeting by the EPR effect, these polyplexes are designed to actively target tumor cells through the chemical attachment of targeting groups to the polyplex periphery. The incorporation of such groups is most often accomplished through end-group functionalization of the PEG block using chemical conjugation techniques. Like viral particles, polyplexes functionalized with targeting groups utilize receptor-ligand interactions to control the spatial distribution of the polyplexses after administration, further enhancing cell-specific delivery of therapeutics. In cancer therapy, targeting groups are designed to interact with receptors that are over-expressed in cancerous tissue relative to normal tissue such as folic acid, oligopeptides, sugars, and monoclonal antibodies. See Pan, D.; Turner, J. L.; Wooley, K. L. Chem. Commun. 2003, 2400-2401; Gabizon, A.; Shmeeda, H.; Horowitz, A. T.; Zalipsky, S. Adv. Drug Deliv. Rev. 2004, 56, 1177-1202; Reynolds, P. N.; Dmitriev, I.; Curiel, D. T. Vector. Gene Ther. 1999, 6, 1336-1339; Derycke, A. S. L.; Kamuhabwa, A.; Gijsens, A.; Roskams, T.; De Vos, D.; Kasran, A.; Huwyler, J.; Missiaen, L.; de Witte, P. A. M. T J. Nat. Cancer Inst. 2004, 96, 1620-30; Nasongkla, N., Shuai, X., Ai, H.,; Weinberg, B. D. P., J.; Boothman, D. A.; Gao, J. Angew. Chem. Int. Ed. 2004, 43, 6323-6327; Jule, E.; Nagasaki, Y.; Kataoka, K. Bioconj. Chem. 2003, 14, 177-186; Stubenrauch, K.; Gleiter, S.; Brinkmann, U.; Rudolph, R.; Lilie, H. Biochem. J. 2001, 356, 867-873; Kurschus, F. C.; Kleinschmidt, M.; Fellows, E.; Dornmair, K.; Rudolph, R.; Lilie, H.; Jenne, D. E. FEBS Lett. 2004, 562, 87-92; and Jones, S. D.; Marasco, W. A. Adv. Drug Del. Rev. 1998, 31, 153-170.

The R^{b} moiety can be used to attach targeting groups for cell specific delivery including, but not limited to, proteins, oliogopeptides, antibodies, monosaccarides, oligosaccharides, vitamins, or other small biomolecules. Such targeting groups include, but or not limited to monoclonal and polyclonal antibodies (e.g. IgG, IgA, IgM, IgD, IgE antibodies), sugars (e.g. mannose, mannose-6-phosphate, galactose), proteins (e.g. Transferrin), oligopeptides (e.g. cyclic and acylic RGD-containing oligopedptides), and vitamins (e.g. folate).

In other embodiments, the R^{b} moiety of any of Formulae III, III-a, III-b, or IV is conjugated to biomolecules which promote cell entry and/or endosomal escape. Such biomolecules include, but are not limited to, oligopeptides containing protein transduction domains such as the HIV Tat peptide sequence (GRKKRRQRRR) or oligoarginine (RRRRRRRRR). Oligopeptides which undergo conformational changes in varying pH environments such oligohistidine (HHHHH) also promote cell entry and endosomal escape.

Compounds having R^{b} moieties suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the R^{b} groups of a compound of Formulae III, III-a, III-b, or IV to a macromolecule via Click chemistry or metal free click chemistry.

According to one embodiment, the R^{b} moiety is an azide-containing group. According to another embodiment, the R^{b} moiety is an alkyne-containing group. In certain embodiments, the R^{b} moiety has a terminal alkyne moiety. In other embodiments, the R^{b} moiety is an alkyne moiety having an electron withdrawing group. Accordingly, in such embodiments, the R^{b} moiety is

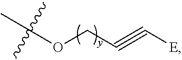

wherein E is an electron withdrawing group and y is 0-6. Such electron withdrawing groups are known to one of ordinary skill in the art. In certain embodiments, E is an ester. In other embodiments, the R^{b} moiety is

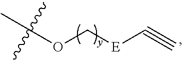

wherein E is an electron withdrawing group, such as a —C(O)O— group and y is 0-6.

In other embodiments, the R^{b} moiety is suitable for metal free click chemistry (also known as copper free click chemistry). Examples of such chemistries include cyclooctyne derivatives (Codelli, et. al. J. Am. Chem. Soc., 2008, 130, 11486-11493; Jewett, et. al. J. Am. Chem. Soc., 2010, 132, 3688-3690; Ning, et. al. Angew. Chem. Int. Ed., 2008, 47, 2253-2255), difluoro-oxanorbornene derivatives (van Berkel, et. al. Chem Bio Chem, 2007, 8, 1504-1508), or nitrile oxide derivatives (Lutz, et. al. Macromolecules, 2009, 42, 5411-5413). Without wishing to be bound by any particular theory, it is believed that the use of metal free click conditions offers certain advantages for the encapsulation of polynucleotides. Such functionalized PEG derivatives suitable for metal free click chemistry are described in detail in U.S. Ser. No. 61/312,842, filed Mar. 11, 2010, the entirety of which is hereby incorporated herein by reference.

Certain metal-free click moieties are known in the literature. Examples include 4-dibenzocyclooctynol (DIBO)

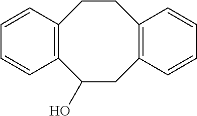

(from Ning et. al; Angew Chem Int Ed, 2008, 47, 2253); difluorinated cyclooctynes (DIFO or DFO)

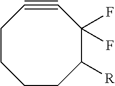

(from Codelli, et. al.; J. Am. Chem. Soc. 2008, 130, 11486-11493.); or biarylazacyclooctynone (BARAC)

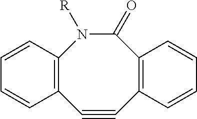

(from Jewett et. al.; J. Am. Chem. Soc. 2010, 132, 3688).

In certain embodiments, the present invention provides a targeted PEG-conjugated cationic polymer of formula V or a salt thereof:

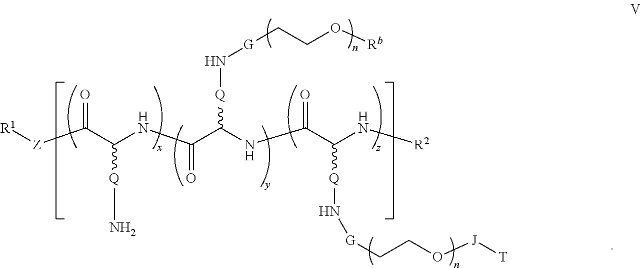

wherein each of R¹, Q, Z, G, x , y, n, R^{b} and R² is as defined above and as described in classes and subclasses herein, both singly and in combination;
   z is 1-200;
   each J is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   each T is independently a targeting group.

In certain embodiments, z is about 1 to about 200. In certain embodiments, z is about 25. In certain embodiments, z is about 10. In certain embodiments, z is about 20. In certain embodiments, z is about 15. In other embodiments z is about 1 to about 25. In other embodiments, z is about 50. According to yet another embodiment, z is about 25-75. In other embodiments, z is about 100. In other embodiments, z is selected from 10±5, 15±5, 25±5, 50±5, 75±10, 100±10, or 125±10.

In certain embodiments, the J group is a valence bond as described above. In certain embodiments, the J group is a methylene group. In other embodiments, the J group is a carbonyl group. In certain embodiments, the J group of Formula V-a is a valence bond. In other embodiments, the J group is represented by a moiety in Table 7.

TABLE 7

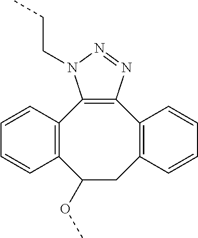

aa

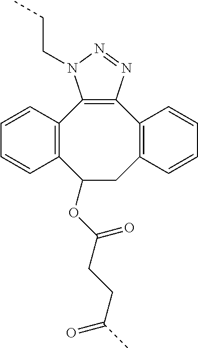

bb

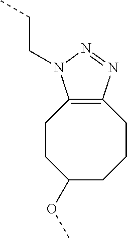

cc

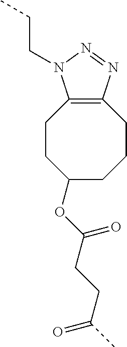

dd

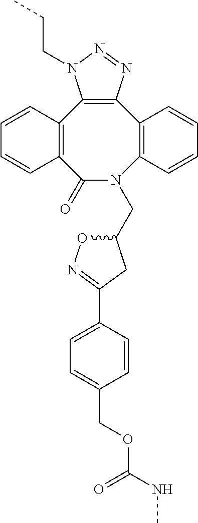

ee

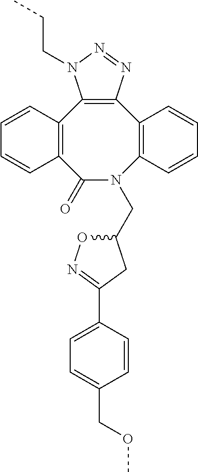

gg

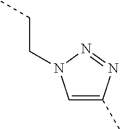

hh

In some embodiments, the present invention provides a cationic polymer of formula V, or a salt thereof, wherein each variable is as defined and described herein, both singly and in combination.

In certain embodiments, the present invention provides a targeted PEG-conjugated polyplex having a polynucleotide encapsulated therein, comprising a cationic polymer of formula V or a salt thereof.

In some embodiments, the present invention provides a polyplex having a polynucleotide encapsulated therein, comprising a cationic polymer of formula V, or a salt thereof, wherein each variable is as defined and described herein, both singly and in combination.

It will be appreciated by one skilled in the art the copolymer of formula V is a mixed, random copolymer comprised of side chain groups containing free amines or ammonium salts; conjugated PEG chains; and conjugated PEG chains with a terminal targeting group moiety. Furthermore, it is understood that x of formula V represents the number of free amines or ammonium salts; that y of formula V represents the number of repeats having pendant PEG chains; and that z of formula V represents the number of repeats that have a pendant PEG chain possessing a terminal targeting group.

In certain embodiments, the present invention provides a method of preparation for a targeted PEG-conjugated polyplex having a polynucleotide encapsulated therein, comprising a cationic polymer of formula V or a salt thereof:

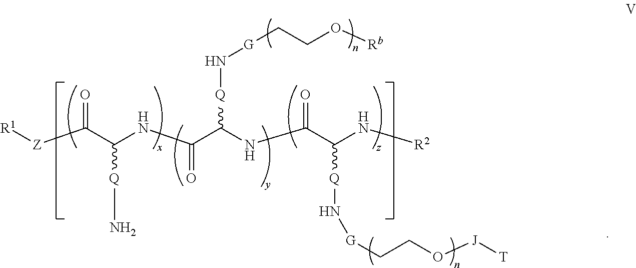

wherein each of R¹, Q, Z, G, x , y, z, n, J, T, R^{b} and R² is as defined above and as described in classes and subclasses herein, both singly and in combination;
from a PEG-conjugated polyplex having a polynucleotide encapsulated therein, comprising a cationic polymer of formula III or a salt thereof:

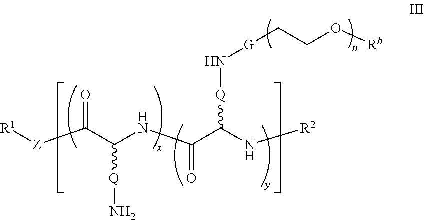

wherein each of R¹, Q, Z, G, x , y, n, R^{b} and R² is as defined above and as described in classes and subclasses herein, both singly and in combination;
   comprising the steps of:
(1) providing a PEG-conjugated polyplex having a polynucleotide encapsulated therein, comprising a cationic polymer of formula III;
(2) performing a Click reaction between the R^{b} group of formula III with a suitable click-ready targeting group to provide the targeted, PEG-conjugated polyplex of Formula V.

In certain embodiments, the present invention provides a targeted PEG-conjugated cationic polymer of formula V-a or a salt thereof:

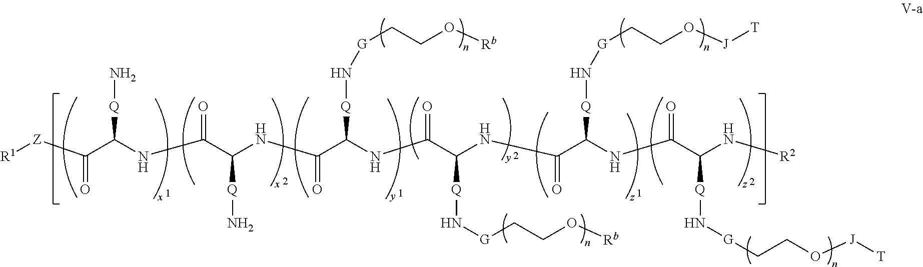

wherein each of R¹, Q, Z, G, x¹, x², y¹, y², z¹, z², n, J, T, R^{b} and R² is as defined above and as described in classes and subclasses herein, both singly and in combination.

In some embodiments, the present invention provides a cationic polymer of formula V-a, or a salt thereof, wherein each variable is as defined and described herein, both singly and in combination.

In certain embodiments, the present invention provides a targeted PEG-conjugated polyplex having a polynucleotide encapsulated therein, comprising a cationic polymer of formula V-a or a salt thereof.

In some embodiments, the present invention provides a polyplex having a polynucleotide encapsulated therein, comprising a cationic polymer of formula V-a, or a salt thereof, wherein each variable is as defined and described herein, both singly and in combination.

In certain embodiments, the present invention provides a method of preparation for a targeted PEG-conjugated polyplex having a polynucleotide encapsulated therein, comprising a cationic polymer of formula V-a or a salt thereof:

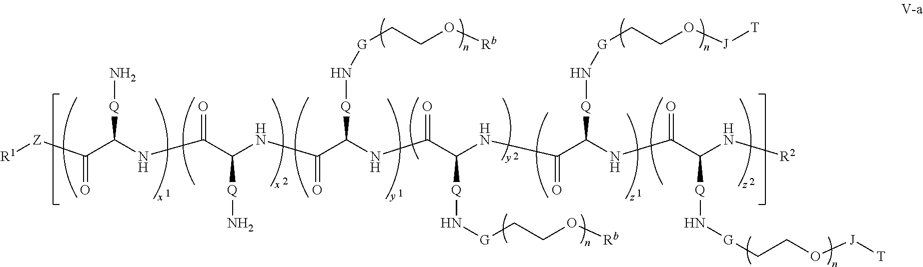

wherein each of R¹, Q, Z, G, x¹, x², y¹, y², z¹, z², n, J, T, R^{b} and R² is as defined above and as described in classes and subclasses herein, both singly and in combination;
   from a PEG-conjugated polyplex having a polynucleotide encapsulated therein, comprising a cationic polymer of formula III-a or a salt thereof:

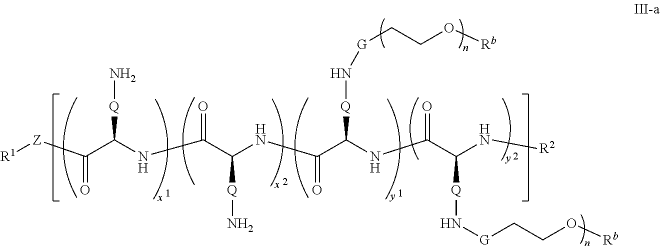

wherein each of R¹, Q, Z, G, x¹, x², y¹, y², n, R^{b} and R² is as defined above and as described in classes and subclasses herein, both singly and in combination;
   comprising the steps of:
(1) providing a PEG-conjugated polyplex having a polynucleotide encapsulated therein, comprising a cationic polymer of formula III-a; and
(2) performing a Click reaction between the R^{b} group of formula III-a with a suitable click-ready targeting group to provide the targeted, PEG-conjugated polyplex of formula V-a.

In certain embodiments, the present invention provides a targeted PEG-conjugated cationic polymer of formula V-b or a salt thereof:

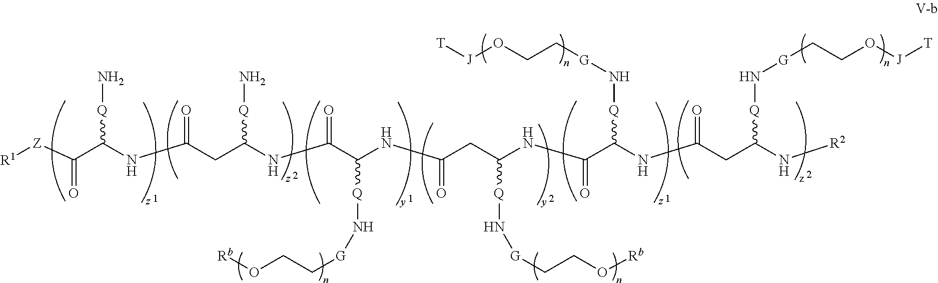

wherein each of R¹, Q, Z, G, x¹, x², y¹, y², z¹, z², n, J, T, R^{b} and R² is as defined above and as described in classes and subclasses herein, both singly and in combination.

In some embodiments, the present invention provides a cationic polymer of formula V-b, or a salt thereof, wherein each variable is as defined and described herein, both singly and in combination.

In certain embodiments, the present invention provides a targeted PEG-conjugated polyplex having a polynucleotide encapsulated therein, comprising a cationic polymer of formula V-b or a salt thereof:

In some embodiments, the present invention provides a polyplex having a polynucleotide encapsulated therein, comprising a cationic polymer of formula V-b, or a salt thereof, wherein each variable is as defined and described herein, both singly and in combination.

In certain embodiments, the present invention provides method of preparation for a targeted PEG-conjugated polyplex having a polynucleotide encapsulated therein, comprising a cationic polymer of formula V-b or a salt thereof:

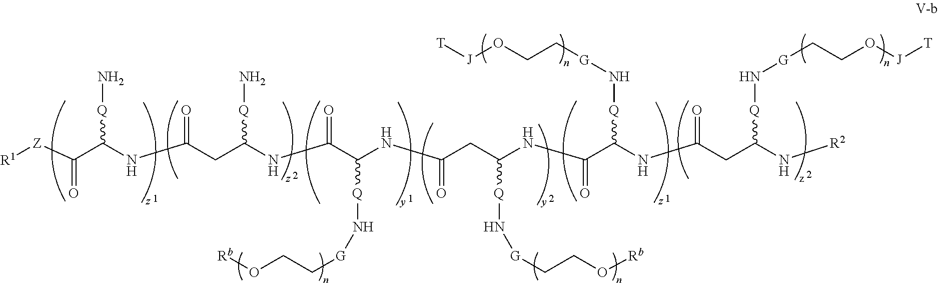

wherein each of R¹, Q, Z, G, x¹, x², y¹, y², z¹, z², n, J, T, R^{b} and R² is as defined above and as described in classes and subclasses herein, both singly and in combination.
   from a PEG-conjugated polyplex having a polynucleotide encapsulated therein, comprising a cationic polymer of formula III-b or a salt thereof:

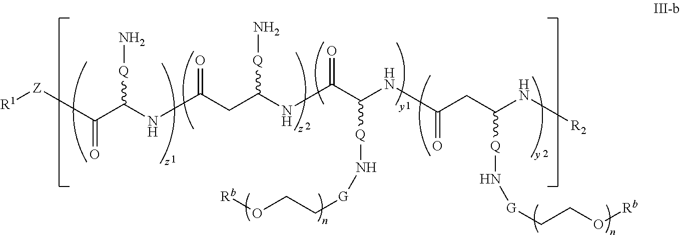

wherein each of R¹, Q, Z, G, z¹, z², y¹, y², n, R^{b} and R² is as defined above and as described in classes and subclasses herein, both singly and in combination;
   comprising the steps of:
(1) providing a PEG-conjugated polyplex having a polynucleotide encapsulated therein, comprising a cationic polymer of formula III-b; and
(2) performing a Click reaction between the R^{b} group of formula III-b with a suitable click-ready targeting group to provide the targeted, PEG-conjugated polyplex of Formula V-b.

It will be appreciated by one skilled in the art the each of the copolymers of formulae V-a and V-b is a mixed, random copolymer comprised of side chain groups containing free amines or ammonium salts; conjugated PEG chains; and conjugated PEG chains with a terminal targeting group moiety. Furthermore, it is understood that x¹ and x² of formulae V-a and V-b represent the number of free amines or ammonium salts; that y¹ and y² of formulae V-a and V-b represent the number of repeats having pendant PEG chains; and that z¹ and z² of formulae V-a and V-b represent the number of repeats that have a pendant PEG chain possessing a terminal targeting group.

As generally described above, a suitable click-ready targeting group is comprised of a targeting group conjugated to a moiety capable of undergoing click chemistry. Such targeting groups are described in detail in United States patent application publication number 2009/0110662, published Apr. 30, 2009, the entirety of which is hereby incorporated by reference.

In certain embodiments, the present invention provides a targeted, PEG-conjugated cationic polymer of formula VI or a salt thereof:

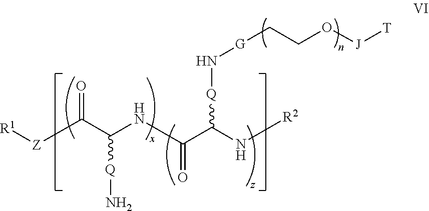

wherein each of R¹, Q, Z, G, x, z, n, J, T, and R² is as defined above and as described in classes and subclasses herein, both singly and in combination.

In some embodiments, the present invention provides a cationic polymer of formula VI, or a salt thereof, wherein each variable is as defined and described herein, both singly and in combination.

In certain embodiments, the present invention provides a targeted, PEG-conjugated polyplex having a polynucleotide encapsulated therein, comprising a cationic polymer of formula VI or a salt thereof.

In some embodiments, the present invention provides a polyplex having a polynucleotide encapsulated therein, comprising a cationic polymer of formula VI, or a salt thereof, wherein each variable is as defined and described herein, both singly and in combination.

In certain embodiments, the present invention provides a method of preparing a PEG-conjugated polyplex having a polynucleotide encapsulated therein, comprising a cationic polymer of formula VI or a salt thereof:

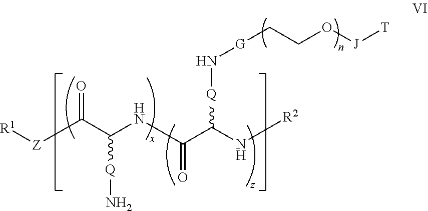

wherein each of R¹, Q, Z, G, x, z, n, J, T, and R² is as defined above and as described in classes and subclasses herein, both singly and in combination;
   from a compound of formula VII:

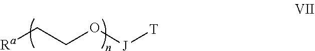

wherein each of R^{a}, J, n, and T is as defined above and as described in classes and subclasses herein, both singly and in combination;
   comprising the steps of:
(1) providing a polyplex having a polynucleotide encapsulated therein, comprising a cationic polymer of formula I, as defined above and described in classes and subclasses herein;
(2) optionally adjusting the pH of the polyplex solution to pH 4.0-9.0; and
(3) conjugating the PEG to the polyplex by the reaction of an electrophile of formula VII and an amine group of formula I to afford a cationic polymer of formula VI.

In certain embodiments, the present invention provides a targeted, PEG-conjugated cationic polymer of formula VI-a or a salt thereof:

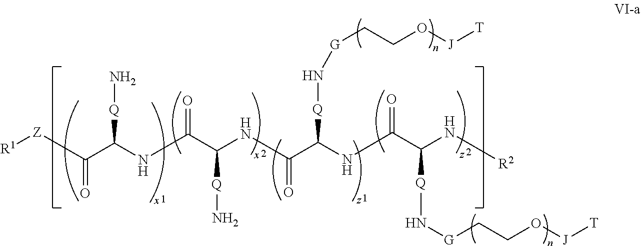

wherein each of R¹, Q, Z, G, x¹, x², z¹, z²₅ n, J, T, and R² is as defined above and as described in classes and subclasses herein, both singly and in combination.

In some embodiments, the present invention provides a polyplex having a polynucleotide encapsulated therein, comprising a cationic polymer of formula VI-a, or a salt thereof, wherein each variable is as defined and described herein, both singly and in combination.

In certain embodiments, the present invention provides a targeted, PEG-conjugated polyplex having a polynucleotide encapsulated therein, comprising a cationic polymer of formula VI-a or a salt thereof

In some embodiments, the present invention provides a polyplex having a polynucleotide encapsulated therein, comprising a cationic polymer of formula VI-a, or a salt thereof, wherein each variable is as defined and described herein, both singly and in combination.

In certain embodiments, the present invention provides method of preparation for a PEG-conjugated polyplex having a polynucleotide encapsulated therein, comprising a cationic polymer of formula VI-a or a salt thereof:

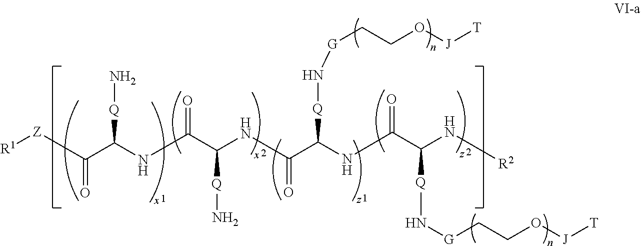

wherein each of R¹, Q, Z, G, x¹, x², z¹, z², n, J, T, and R² is as defined above and as described in classes and subclasses herein, both singly and in combination;
from a compound of formula VII:

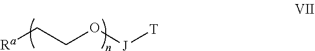

wherein each of R^{a}, J, n, and T is as defined above and as described in classes and subclasses herein, both singly and in combination.
   comprising the steps of:
(1) providing a polyplex having a polynucleotide encapsulated therein, comprising a cationic polymer of formula I-a, as defined above and described in classes and subclasses herein;
(2) optionally adjusting the pH of the polyplex solution to pH 4.0-9.0; and
(3) conjugating the PEG to the polyplex by the reaction of the electrophile of formula VII and an amine group of formula I-a to afford a cationic polymer of formula VI-a.

In certain embodiments, the present invention provides a targeted, PEG-conjugated cationic polymer of formula VI-b or a salt thereof:

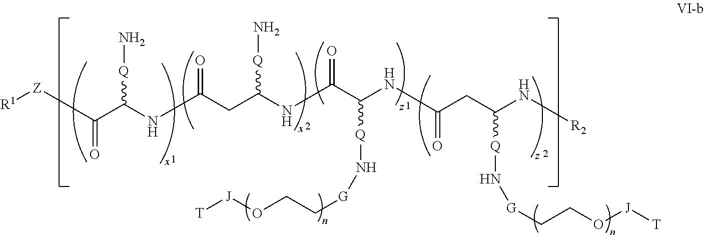

wherein each of R¹, Q, Z, G, x¹, x², z¹, z², n, J, T, and R² is as defined above and as described in classes and subclasses herein, both singly and in combination.

In some embodiments, the present invention provides a cationic polymer of formula VI-b, or a salt thereof, wherein each variable is as defined and described herein, both singly and in combination.

In certain embodiments, the present invention provides a targeted, PEG-conjugated polyplex having a polynucleotide encapsulated therein, comprising a cationic polymer of formula VI-b or a salt thereof.

In some embodiments, the present invention provides a polyplex having a polynucleotide encapsulated therein, comprising a cationic polymer of formula VI-b, or a salt thereof, wherein each variable is as defined and described herein, both singly and in combination.

In certain embodiments, the present invention provides method of preparation for a PEG-conjugated polyplex having a polynucleotide encapsulated therein, comprising a cationic polymer of formula VI-b or a salt thereof:

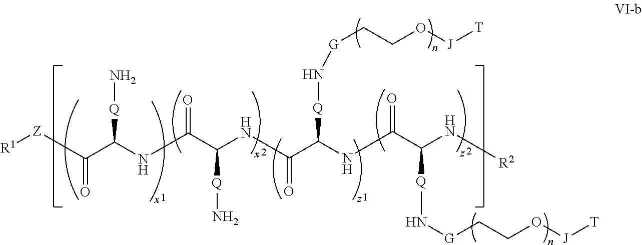

wherein each of R¹, Q, Z, G, x¹, x², z¹, z², n, J, T, and R² is as defined above and as described in classes and subclasses herein, both singly and in combination;
from a compound of formula VII:

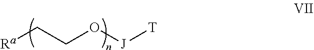

wherein each of R^{a}, J, n, and T is as defined above and as described in classes and subclasses herein, both singly and in combination;
   comprising the steps of:
(1) providing a polyplex having a polynucleotide encapsulated therein, comprising a cationic polymer of formula I-b, as defined above and described in classes and subclasses herein;
(2) optionally adjusting the pH of the polyplex solution to pH 4.0-9.0; and
(3) conjugating the PEG to the polyplex by the reaction of an electrophile of Formula VII and an amine group of Formula I-b to afford a cationic polymer of Formula VI-b.

In some embodiments, the present invention provides a composition comprising a compound of formula VI and at least one compound selected from a compound of formula VI-a and/or VI-b.

In certain embodiments, the present invention provides a polyplex having a polynucleotide encapsulated therein, wherein the polyplex comprises a compound of formula VI and at least one compound selected from a compound of formula VI-a and/or VI-b.

In certain embodiments, the T group of is a targeting group as described above. In some embodiments, the T group is an EGFR targeting peptide. In some embodiments, the T group is transferrin. In other embodiments, the T group is an EGF protein, or fragment thereof.

### 4. Uses, Methods, and Compositions

As described herein, polyplexes of the present invention can encapsulate a wide variety of therpaeutic agents useful for treating a wide variety of diseases. In certain embodiments, the present invention provides a nucleotide-loaded polyplex, as described herein, wherein said polyplex is useful for treating the disorder for which the nucleotide is known to treat. According to one embodiment, the present invention provides a method for treating one or more disorders selected from pain, inflammation, arrhythmia, arthritis (rheumatoid or osteoarthritis), atherosclerosis, restenosis, bacterial infection, viral infection, depression, diabetes, epilepsy, fungal infection, gout, hypertension, malaria, migraine, cancer or other proliferative disorder, erectile dysfunction, a thyroid disorder, neurological disorders and hormone-related diseases, Parkinson's disease, Huntington's disease, Alzheimer's disease, a gastro-intestinal disorder, allergy, an autoimmune disorder, such as asthma or psoriasis, osteoporosis, obesity and comorbidities, a cognitive disorder, stroke, AIDS-associated dementia, amyotrophic lateral sclerosis (ALS, Lou Gehrig's disease), multiple sclerosis (MS), schizophrenia, anxiety, bipolar disorder, tauopothy, a spinal cord or peripheral nerve injury, myocardial infarction, cardiomyocyte hypertrophy, glaucoma, an attention deficit disorder (ADD or ADHD), a sleep disorder, reperfusion/ischemia, an angiogenic disorder, or urinary incontinence, comprising administering to a patient a PEG-conjugated polyplex, wherein said polyplex encapsulates a therapeutic agent suitable for treating said disorder.

In certain embodiments, the present invention provides a method for treating one or more disorders selected from autoimmune disease, an inflammatory disease, a metabolic disorder, a psychiatric disorder, diabetes, an angiogenic disorder, tauopothy, a neurological or neurodegenerative disorder, a spinal cord injury, glaucoma, baldness, or a cardiovascular disease, comprising administering to a patient an optionally targeted, PEG-covered polyplex wherein said polyplex encapsulates a therapeutic polynucleotide suitable for treating said disorder.

In certain embodiments, nucleotide-loaded polyplexes of the present invention are useful for treating cancer. Accordingly, another aspect of the present invention provides a method for treating cancer in a patient comprising administering to a patient an optionally targeted, PEG-covered polyplex wherein said polyplex encapsulates a therapeutic polynucleotide suitable for treating said cancer. In certain embodiments, the present invention relates to a method of treating a cancer selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia, comprising administering a polyplex in accordance with the present invention wherein said polyplex encapsulates a therapeutic polynucleotide suitable for treating said cancer.

Compositions

In certain embodiments, the invention provides a composition comprising a polyplex of this invention or a pharmaceutically acceptable derivative thereof and a pharmaceutically acceptable carrier, adjuvant, or vehicle. In certain embodiments, a composition of this invention is formulated for administration to a patient in need of such composition. In certain embodiments, a composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In certain embodiments, pharmaceutically acceptable compositions of the present invention are enterically coated.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the drug can be administered to a patient receiving these compositions.

It will be appreciated that dosages typically employed for the encapsulated drug are contemplated by the present invention. In certain embodiments, a patient is administered a drug-loaded polyplex of the present invention wherein the dosage of the drug is equivalent to what is typically administered for that drug. In other embodiments, a patient is administered a drug-loaded polyplex of the present invention wherein the dosage of the drug is lower than is typically administered for that drug.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In order that the invention described herein may be more fully understood, the following examples are set forth. It will be understood that these examples are for illustrative purposes only and are not to be construed as limiting this invention in any manner.

### EXAMPLES

### Example 1

### Preparation of Bifunctional PEGs of the Present Invention

As described generally above, multiblock copolymers of the present invention are prepared using the heterobifunctional PEGs described herein and in U.S. patent application Ser. No. 11/256,735, filed Oct. 24, 2005, published as WO2006/047419 on May 4, 2006 and published as US 20060142506 on Jun. 29, 2006, the entirety of which is hereby incorporated herein by reference. The preparation of multiblock polymers in accordance with the present invention is accomplished by methods known in the art, including those described in detail in U.S. patent application Ser. No. 11/325,020, filed Jan. 4, 2006, published as WO2006/74202 on Jul. 13, 2006 and published as US 20060172914 on Aug. 3, 2006, the entirety of which is hereby incorporated herein by reference.

### Example 2

### Gel Retardation Experiments

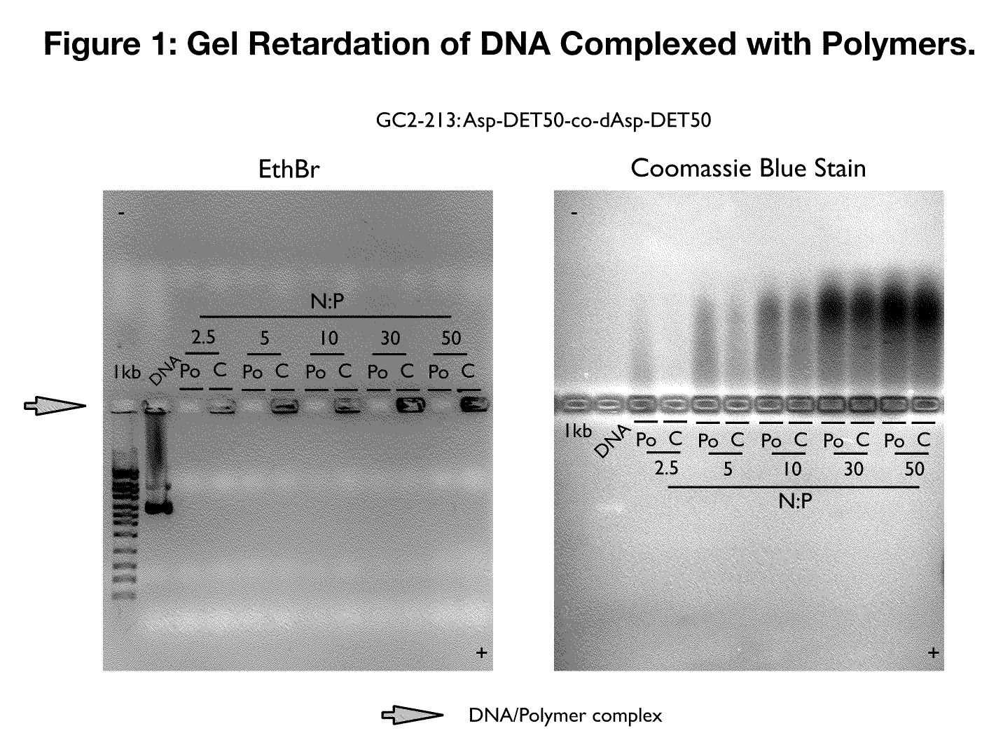

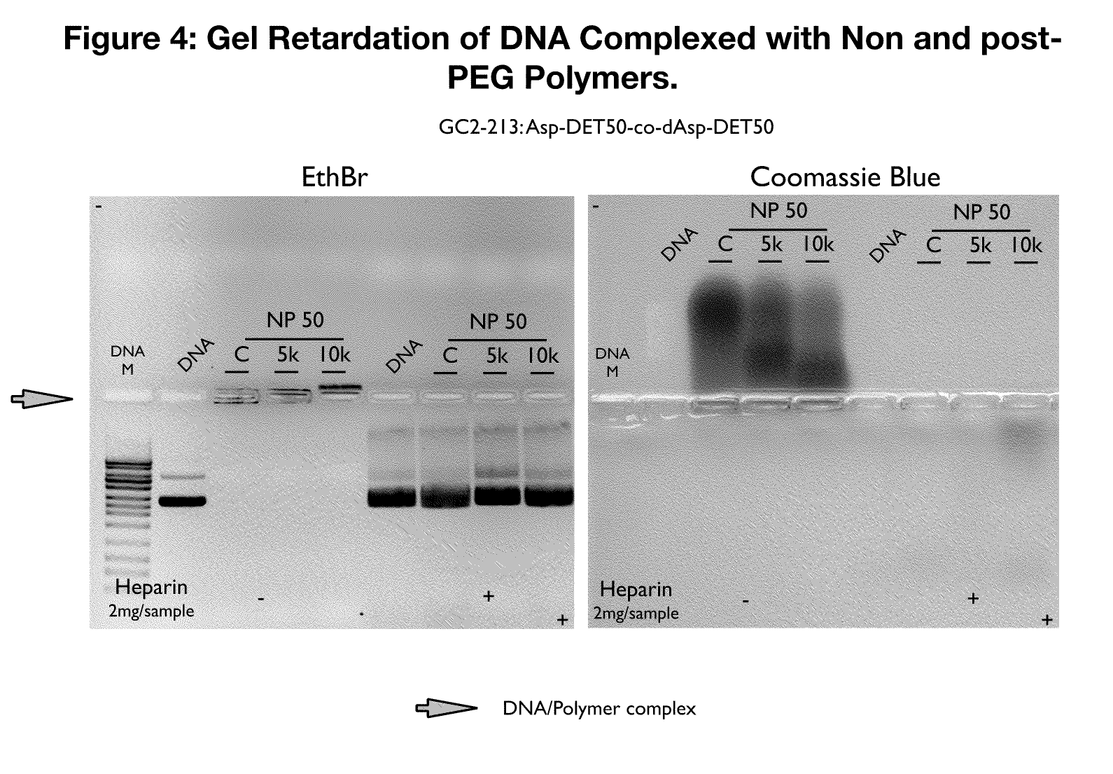

Poly(D/L Asp-DET)/DNA polyplexes were prepared by adding equal volumes of Poly(D/L Asp-DET) solution (dissolved in dH₂O and filter sterilized using a 0.22 μm PES filter) and plasmid DNA solution (200 μg/mL in dH₂O) at the appropriate N:P ratio. Polymer was added to DNA solution, for a final volume of 200 μL, and incubated at room temperature for at least 30 min to allow polyplex formation. Gel loading dye was added to each polymer/DNA complex and samples run on a 1% agarose/ethidium bromide gel in 1×TAE Buffer for 30 min at 200V, FIGS. 1 and 4. The agarose/ethidium bromide gel was post-stained with Coomassie blue for 30 min and then destained overnight using H₂O.

### Example 3

### Nucleic Acid/Polymer Complexation and Polyplex Post-PEG Procedure

Polymers were prepared at a N:P ratio of 50 in H₂O, based on a final amount of 20 μg Luciferase plasmid DNA. The polymers were filter sterilized using a 0.22 μm PES filter and then complexed with 100 uL plasmid DNA at N:P ratio 50, in a final volume of 200 μL, for 30 min at room temperature. 0.5 uL of 3.23M KOH was added to the polyplex solution to increase the pH to between 7-8. Fifty uL of 5K or 10K maleimide PEG (60 mg/mL stock solutions) was added to polyplexes and incubated at 37 C with shaking for three hours. Post-PEG polyplexes were resolved on 1% agarose/ethidium bromide gel in 1×TAE Buffer for 30 min at 200V, FIGS. 1 and 4. The agarose/ethidium bromide gel was stained with Coomassie blue for 30 min and destained overnight using H₂O.

### Example 4

### Polymer/DNA Complex Size Analysis

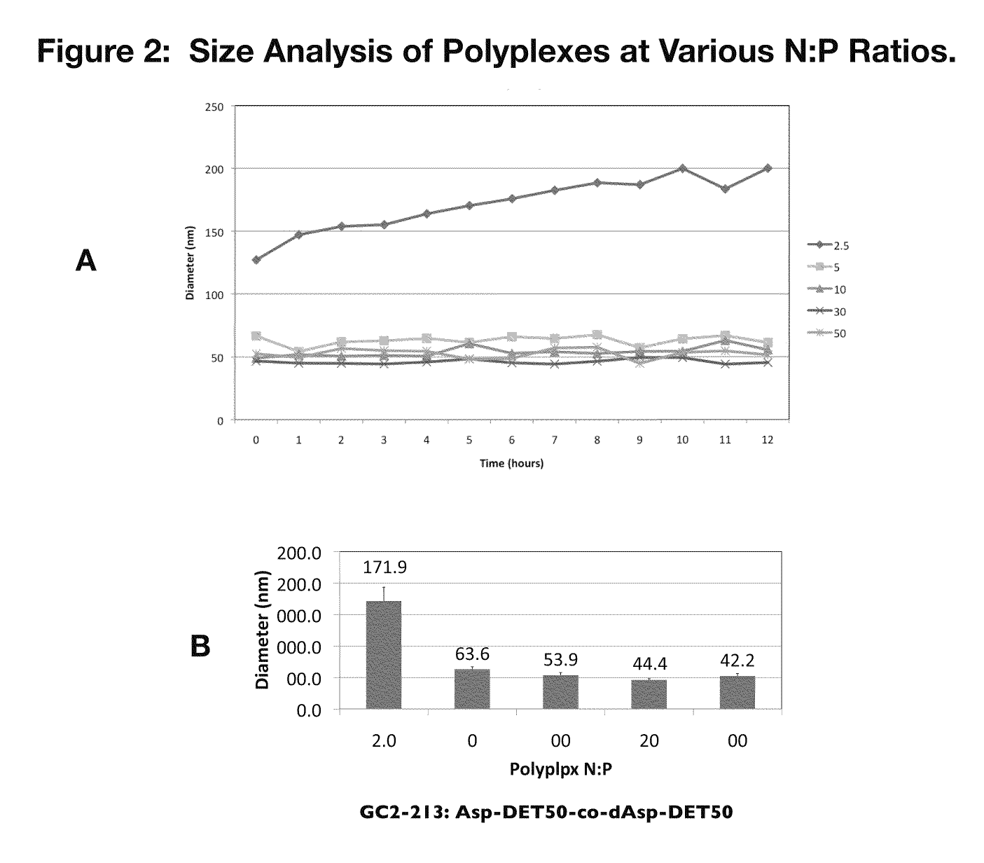

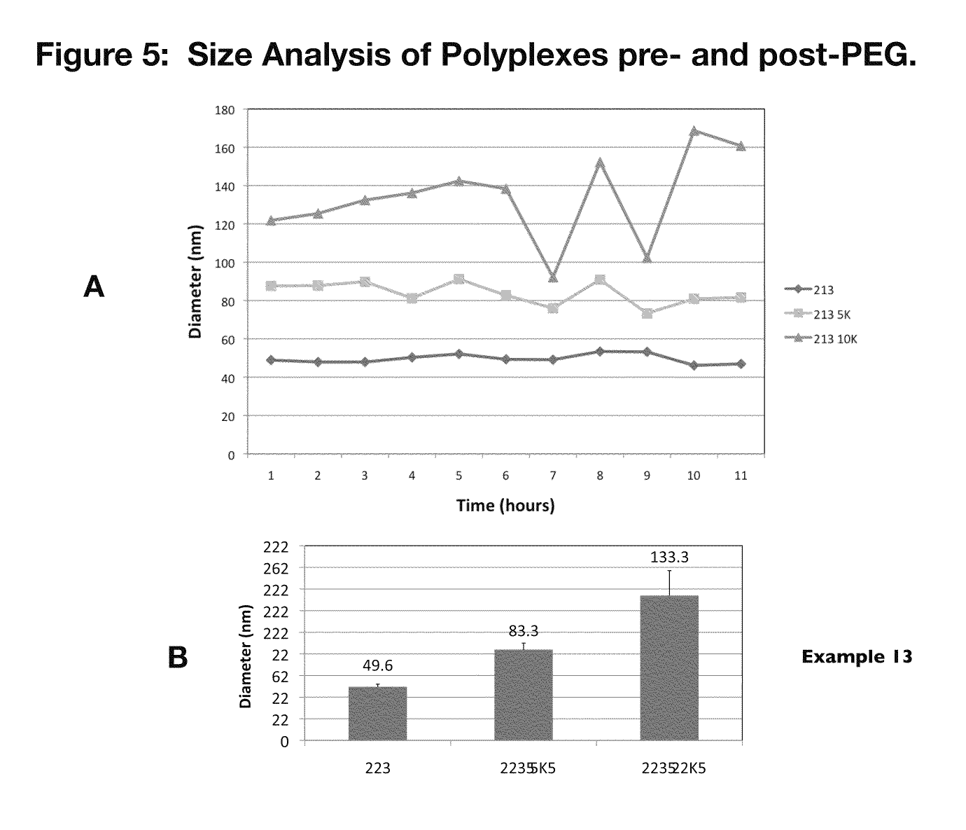

Non- and PEG polyplexes were prepared as described above. Dynamic Light Scattering analysis was performed using a DynaPro Dynamic Light Scattering Plate Reader (Wyatt Technology Corporation, Santa Barbara, Calif.). One hundred and twenty uL of each sample was loaded into a 96 well plate and sizes determined every hour with ten 30 sec acquisitions at 37° C., FIGS. 2 and 5.

### Example 5

### Polymer Titration Experiments

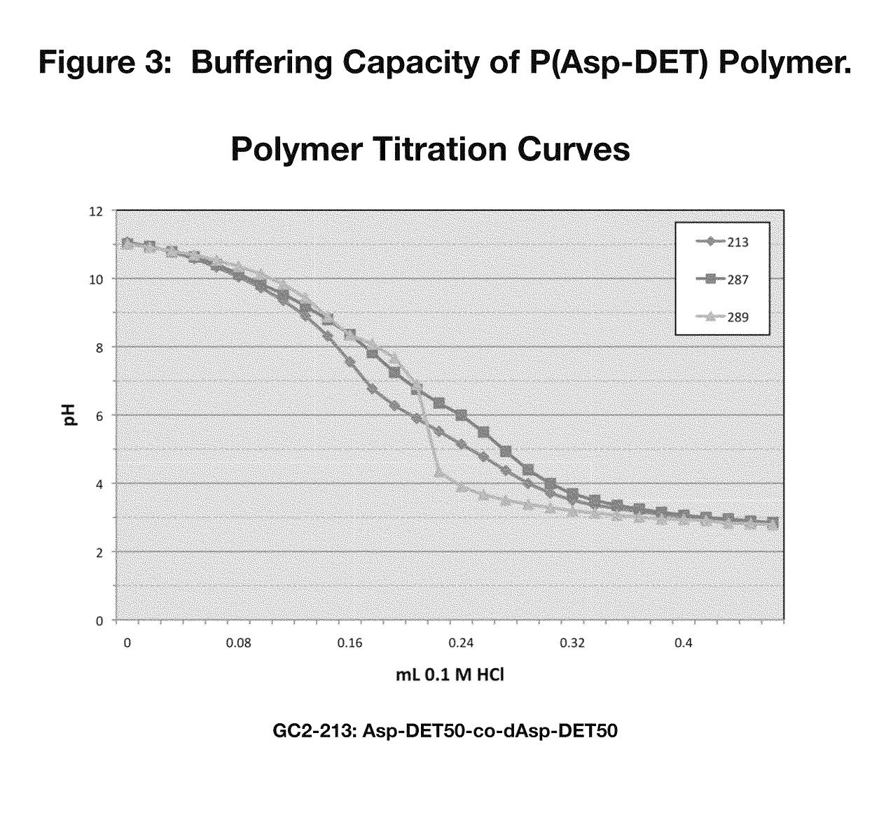

Three mg of polymer was diluted in a 10 mL final volume of 150 mM NaCl. The polymer solution was titrated with 1N HCl and plotted as a function of pH, FIG. 3.

### Example 6

### TEM

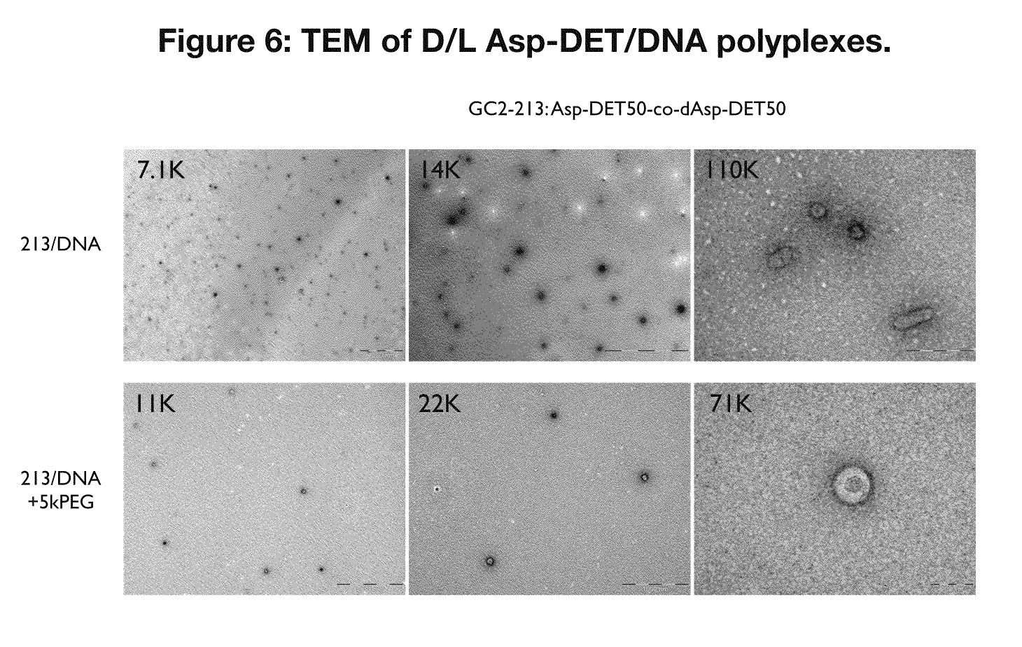

Non- and PEG polyplexes were prepared as described above. Five uL of each sample was spotted onto formvar grids for 1-5 min, washed with H₂O, incubated with 5% uranyl acetate for 1 min and washed again in H₂O. Images were taken using a Morgagni 268D electron microscope, FIG. 6.

### Example 7

### Erythrocyte Aggregation Assay

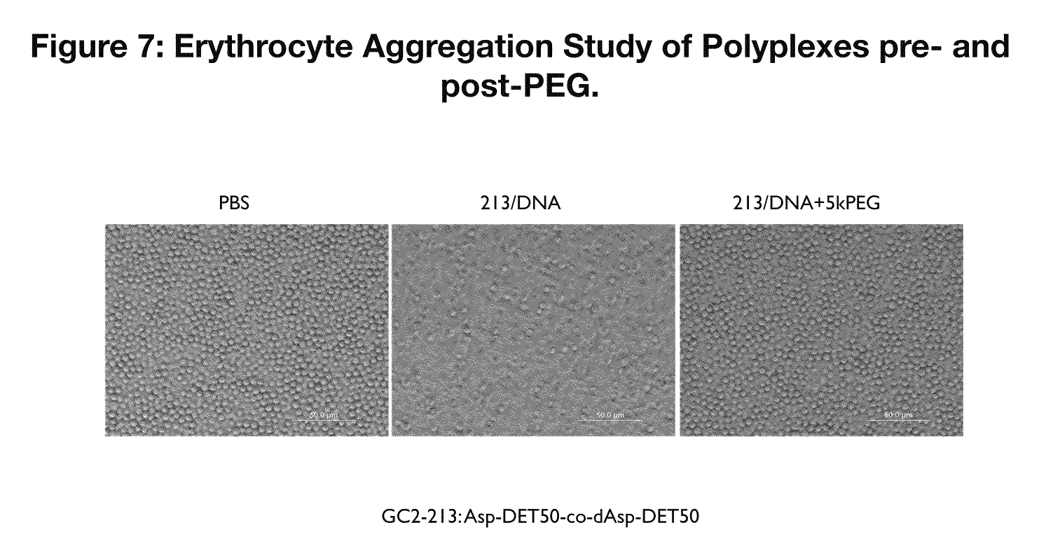

Non- and PEG polyplexes were prepared as described above. Thirty μL of each sample was spiked with 5M NaCl for final 150 mM concentration. Samples were then incubated with erythrocytes (60 uL) in 96 well plates and incubated at 37° C. for 1 hour, FIG. 7.

### Example 8

### Transfections and Plasmid Visualization Experiments

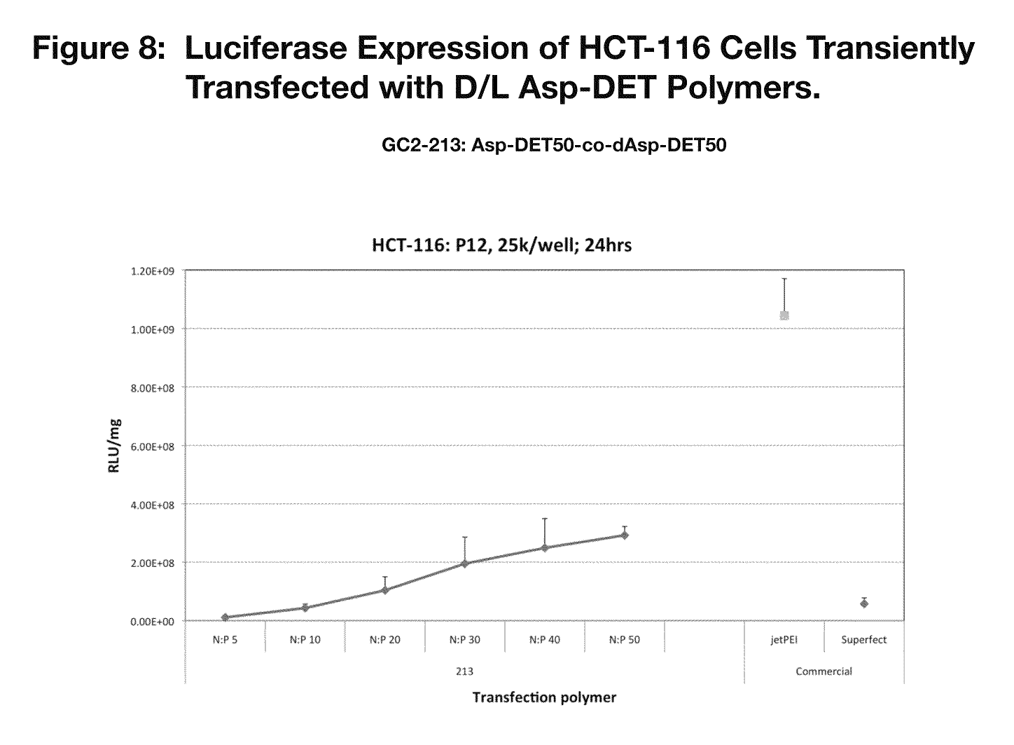

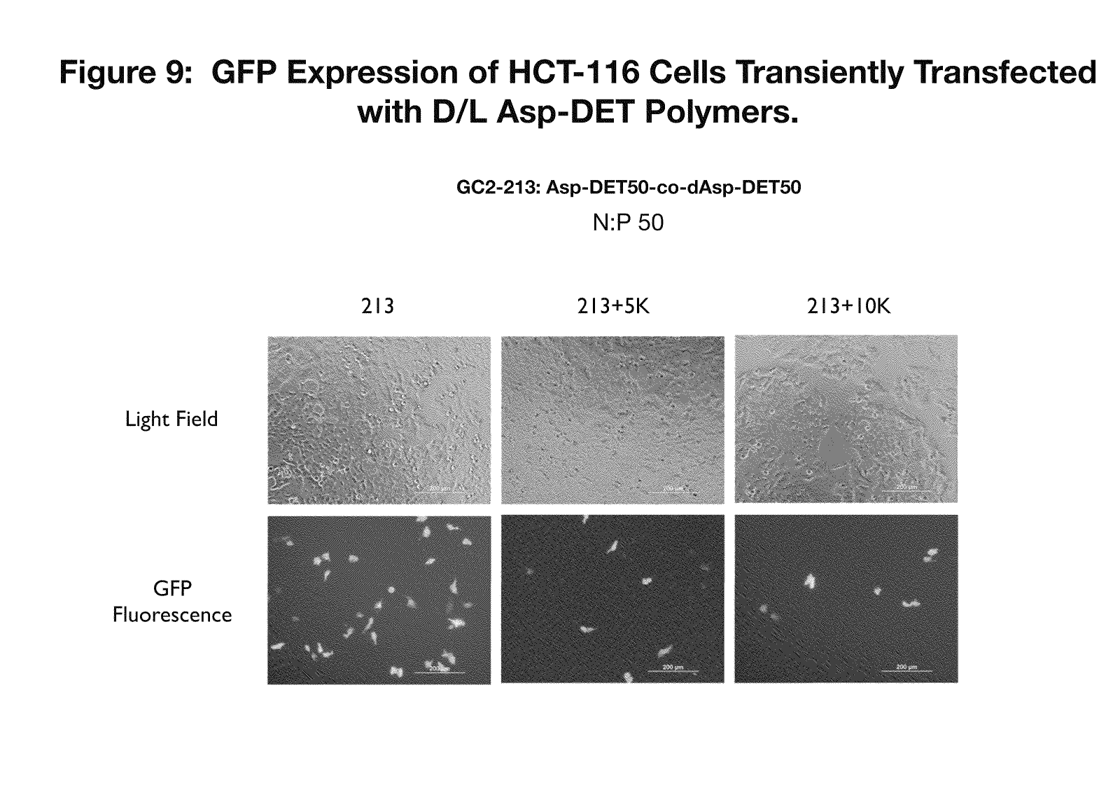

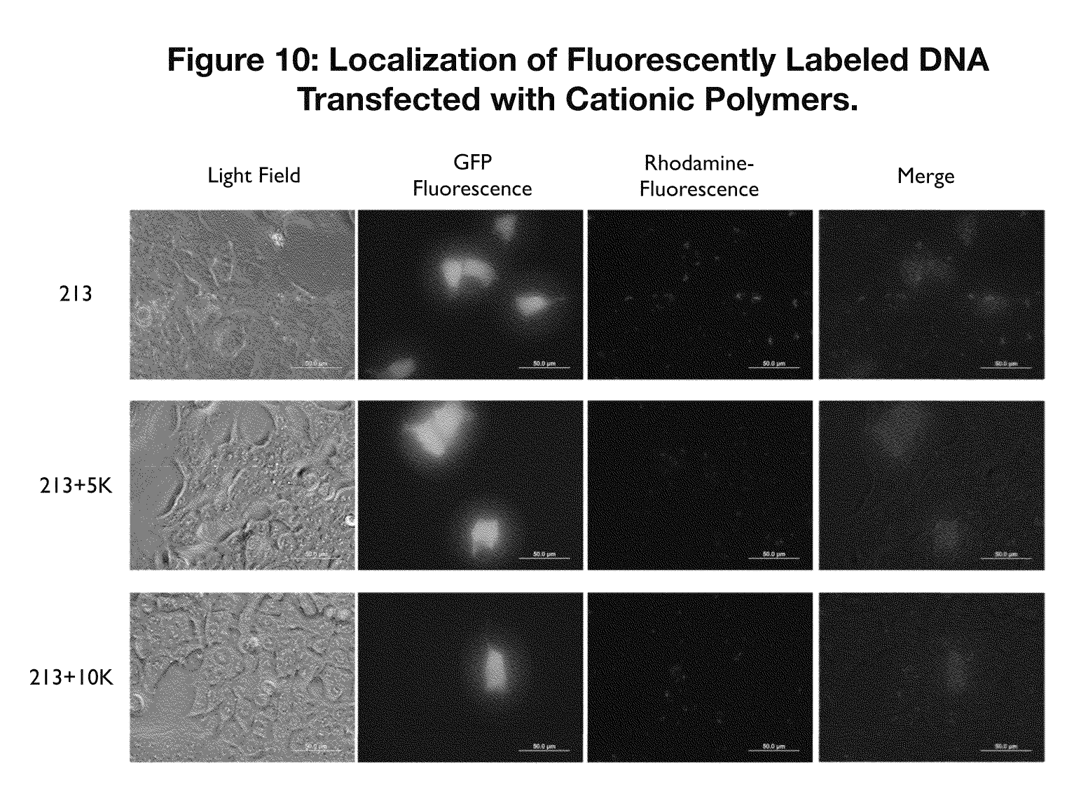

HCT-116 colon cancer cells, obtained from ATCC, were maintained in McCoy's media supplemented with 10% FBS, 2 mM L-glutamine, and 100 units/mL penicillin/streptomycin. Twenty-five thousand HCT-116 cells, in a total volume of 100 μL McCoy's media, were seeded in each well of a 96-well format plate the day before transfection. On the day of transfection, non- and PEG polyplexes were prepared as described above. HCT-116 cells were transfected with either an EGFP plasmid (pZs-Green; Clontech, Mountain View, Calif.) or pGL4-luciferase plasmid, (Promega, Madison, Wis.). Transfection complexes (2.5 μL) were added to the cells and incubated at 37° C. After 24 hours incubation, the cells were either visualized with an Olympus IX71 microscope or luciferase activity was determined using a standard luciferase assay kit (Promega). Protein quantitation was also determined using the Bradford Assay (Bio-Rad Labs, Hercules, Calif.). Experiments with the commercially available transfection reagents jetPEI (Polyplus Transfection Inc, New York, N.Y.) and Superfect (Qiagen, Valencia, Calif.) were also performed using the manufacturers' recommended protocols. Furthermore, transfection experiments for each polymer and commercial transfection reagent was performed in triplicate, and the luciferase activity was normalized to the quantity of protein in each well. FIG. 8 shows a comparison of luciferase transfection efficiencies for P[Asp(DET)] versus commercial reagents. FIG. 9 demonstrates transfection of EGFP between P[Asp(DET)] and 5 k and 10K PEG P[Asp(DET)] polymers. For plasmid visulalization experiments, EGFP plasmid (pZs-Green; Clontech, Mountain View, Calif.) was fluorescently labeled with 5-carboxy-X-rhodamine using the Label IT®Tracker™ Kit (Minis, Madison, Wis.). Twenty-four hours after transfection, cells were visualized with an Olympus IX71 microscope, FIG. 10.

### Example 9

### In Vivo Polymer/DNA Delivery Experiments

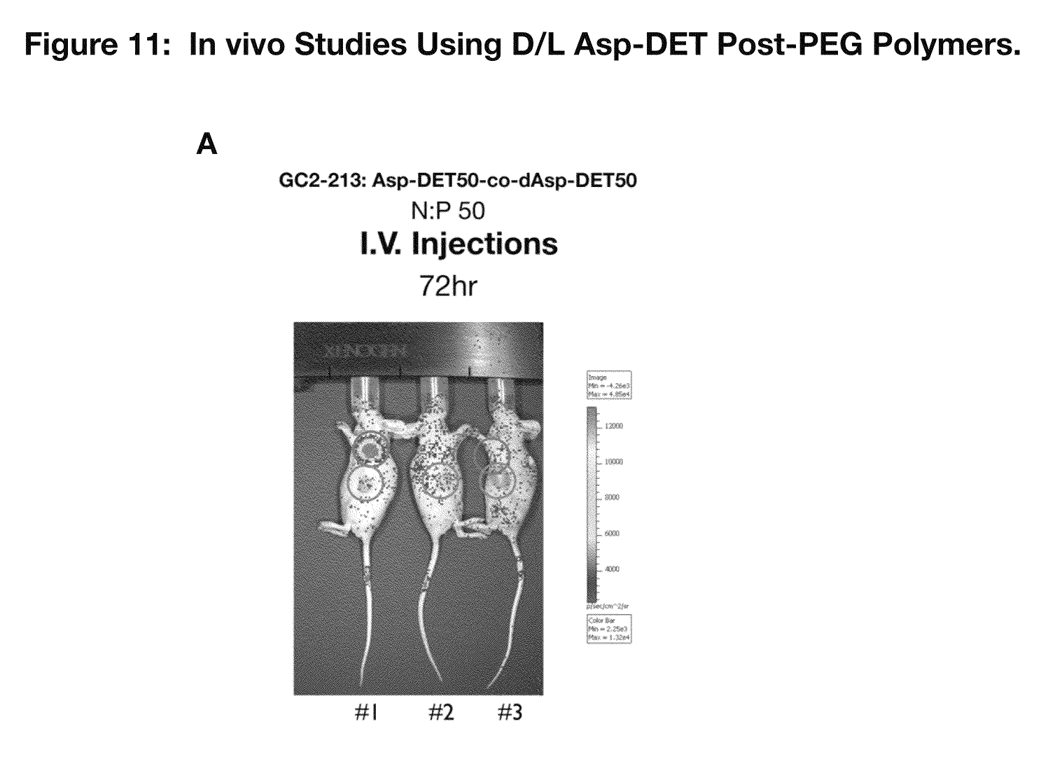

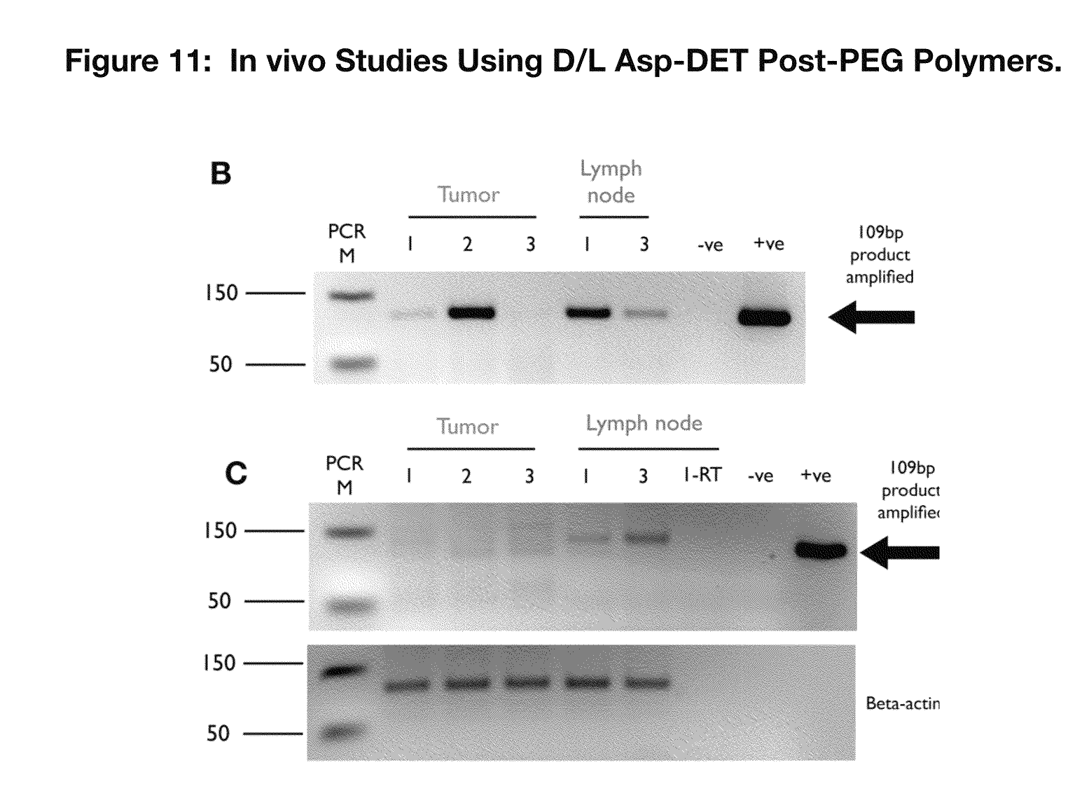

On the day of experiment, 250 uL of PEG polyplexes containing pGL4-luciferase plasmids were prepared as described above. Twenty% glusose was added to samples for a final 5% glucose concentration. The entire glucose/PEG/polyplex sample was administered by tail vein IV administration to tumor bearing nude mice, FIG. 11A. At various time points, mice were anesthetized and imaged using the IVIS Spectrum system (Caliper Life Sciences, Hopkinton, Mass.). At the completion of the experiment, mice were anesthetized, sacrificed by cervical dislocation and various tissues collected. DNA and RNA samples were extracted from tissue samples using the Qiagen AllPrep DNA/RNA Kit. RT-PCR and PCR was performed using pGL4 specific primers, FIG. 11B & C.

### Example 10

### Gel Retardation of DNA Complexed with Polymers

Twenty μg of pGL4 plasmid DNA was complexed with Poly(D/L Asp-DET) at N:P ratios between 2.5 and 50 for 30 min at room temperature. Samples were then resolved on a 1% agarose/ethidium bromide gel FIG. 1. DNA retardation was observed in both DNA/polymers samples at N:P ratios of 2.5. Wells containing intact naked DNA served as controls. Po; polymer only, C; complex, 1 kb; One kb DNA ladder. Agarose/ethidium bromide gels were post-stained with Coomassie blue. Free polymer was detected in all samples with an overall decrease in the amount of free polymer in complexed samples.

### Example 11

### Size Analysis of Polyplexes at Various N:P Ratios

Dynamic light scattering analysis of polyplex size for the D/L polymer between N:P ratios of 2.5 and 50 ranged from ˜170 to 53 nm, FIG. 2A. Time course experiments at 37° C. demonstrated no change in polyplex size for N:P ratios greater than 5, FIG. 2B.

### Example 12

### Buffering Capacity of P[Asp(DET)] Polymer

Asp-DET polymers exhibit buffering capacity within the critical pH buffering area of the curve corresponding to the transition from the endosome to the lysosome (pH 5-7), FIG. 3.

### Example 13

### Gel Retardation of DNA Complexed with Non- and Post-PEG Polymers

Twenty μg of pGL4 plasmid DNA was complexed with Poly(D/L Asp-DET) at N:P 50 for 30 min at room temperature. Polyplexes were the pH adjusted to 7-8 and then incubated with 5 k or 10 k PEG for three hours at 37° C. Samples were then resolved on a 1% agarose/ethidium bromide gel, FIG. 4. DNA retardation was observed in all polyplex samples. Wells containing intact naked DNA served as controls. Po; polymer only, C; complex, 1 kb; One kb DNA ladder. Agarose/ethidium bromide gels were post-stained with Coomassie blue. The degree of PEGylation of free polymer could be determined by Coomassie blue staining of gels.

### Example 14

### Size Analysis of Polyplexes Pre- and Post-PEG

Dynamic light scattering analysis of pre- and post-PEG polyplexes at N:P 50, FIG. 5A. Time course experiments at 37 C demonstrated no change in polyplex sizes for Polyplex alone and 5 k PEG-polyplexes while 10 K PEG-Polyplexes increase in size over time, FIG. 5B.

### Example 15

### TEM of D/L Asp-DET/DNA Polyplexes

P(Asp-DET) polymers interacted with plasmid DNA to form unform and spherical structures which were less than 200 nm in size. Post-PEG polyplexes showed similar morphology and were also smaller than 200 nm, FIG. 6.

### Example 16

### Erythrocyte Aggregation Study of Polyplexes Pre- and Post-PEG

P(Asp-DET)/DNA polyplexes incubated with erythrocytes resulted in extensive cell lysis. In contrast, incubation with post-PEG polyplexes resulted in no change to erythrocytes, similar to the PBS incubated control, FIG. 7.

### Example 17

### Luciferase and GFP Expression of HCT-116 Cells Transiently Transfected with D/L Asp-DET Polymers

HCT-116 cells were transfected in triplicate in 96-well plates with P(Asp-DET) polymers that were complexed with firefly luciferase pGL4 plasmid DNA, at the indicated N:P ratios at a final DNA concentration of 0.25 μg per well. Commercial reagents were used according to the manufacturer's protocol. Twenty-four hr after transfection, luciferase activity for each sample was determined and was normalized to protein content. All results are representative of triplicate experiments. Luciferase activity for D/L mix configuration increased with increased N:P ratios, FIG. 8.

### Example 18

### Luciferase and GFP Expression of HCT-116 Cells Transiently Transfected with D/L Asp-DET Polymers

HCT-116 cells were also transfected in triplicate in 96-well plates with pre and post-PEG polymers that were complexed with a GFP expressing plasmid DNA pZs-Green, N:P 50 ratio at a final DNA concentration of 0.25 μg per well, FIG. 9. Twenty-four hr after transfection, cells were imaged using phase contrast (top panel) and fluorescence for GFP expression (bottom panel), ×10. Cells transfected with the various polyplexes showed little cytotoxicity. Non-PEG polyplexes showed high levels of GFP expression, while 5 k and 10 k post-PEG polyplexes showed lower levels of GFP expression.

### Example 19

### Localization of Fluorescently Labeled DNA Transfected with Cationic Polymers

HCT-116 cells were transfected with pre- and post-PEG polyplexes containing rhodamine-labeled pZs-Green plasmid DNA, FIG. 10. Twenty four hours after transfection, cells were observed by phase contrast (left panel) or fluorescent microscopy (middle panels). Cells expressing pZs-Green GFP protein (green) also contained various amounts of rhodamine-labeled DNA (red) in both the nucleus and cytoplasm. Merged images appear in the right panels. ×40 magnification.

### Example 20

### In Vivo Studies Using D/L Asp-DET Post-PEG Polyplexes

5K Post-PEG[Asp(DET)]/DNA N:P 50 polyplexes, containing 20 μg of pGL4-luciferase plasmid, was administered to HCT-116 tumor bearing nude mice by tail vein administration. IVIS images of mice 72 hours after IV injection, FIG. 11A. HCT-116 tumors are circled red and lymph nodes are circled purple. PCR, FIG. 11B and RT-PCR, FIG. 11C results of tumor and lymph node tissues. Plasmid DNA accumulation was demonstrated in both tumors and lymph nodes while gene expression was observed in lymph nodes.

### Example 21

### In Vivo Studies Using D/L Asp-DET Post-PEG Polyplexes

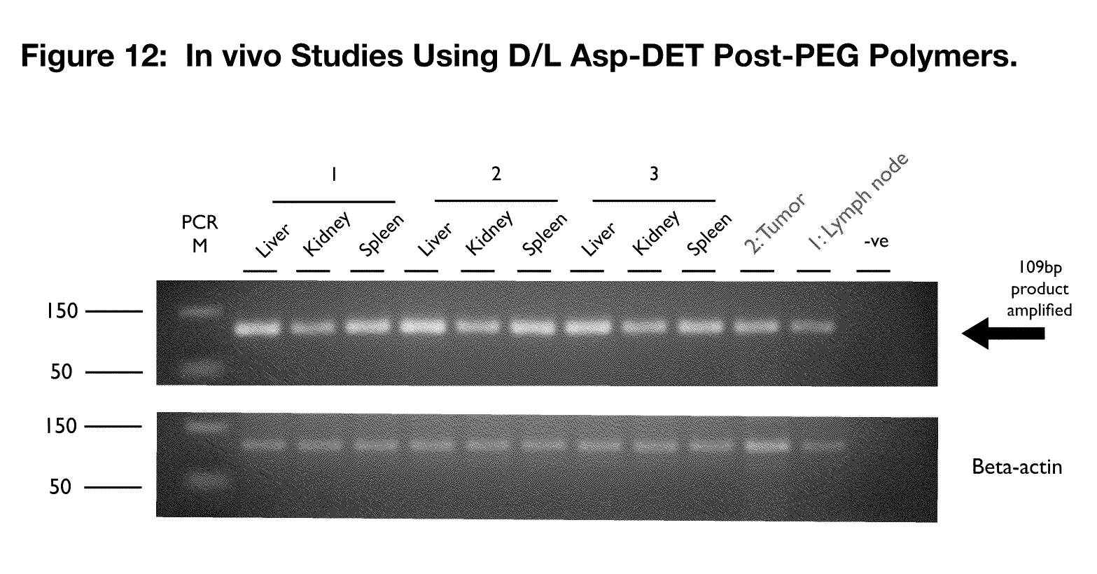

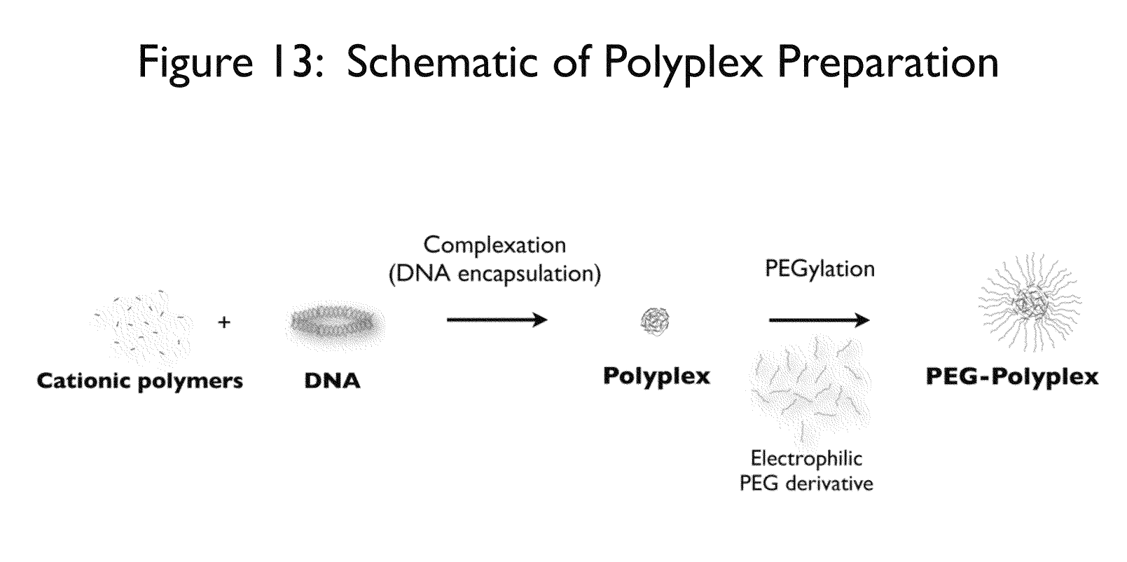

PCR results of various organs from treated nude mice detected high plasmid DNA levels in liver and kidney and moderate levels in spleen, FIG. 12.

### Example 23

### Synthesis of Asp(OBzl)NCA

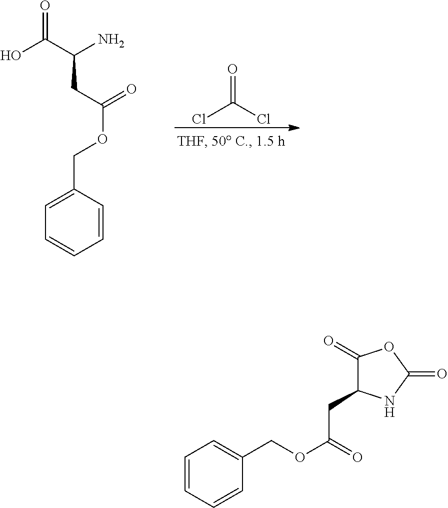

H-Asp(OBzl)-OH (14.0 g, 62.7 mmol) was suspended in 225 mL of anhydrous THF and heated to 50° C. Phosgene (20% in toluene) (40 mL, 80 mmol) was added the amino acid suspension. The amino acid dissolved to give a clear solution over the course of approx. 15 min and was left reacting for another 25 min. The solution was concentrated on the rotovap, the white solid redissolved in a toluene/THF mixture (100 mL/50 mL) and the clear solution rotovaped to dryness. The white solid obtained was redissolved into 100 mL of THF, transferred to a beaker, and dry hexanes were added to precipitate the product. The white solid was isolated by filtration and rinsed twice with dry hexanes (2×200 mL) The NCA was isolated by filtration and dried in vacuo. 14.3 g (65% yield) of Asp(OBzl) NCA was isolated as a white solid. ¹H NMR (d₆-DMSO) δ 9.00 (1H), 7.48-7.25 (5H), 5.13 (2H), 4.69 (1H), 3.09 (1H), 2.92 (1H) ppm

### Example 24

### Synthesis of D-Asp(OBzl)NCA

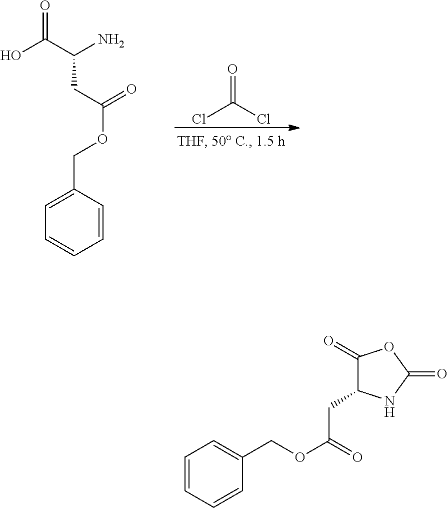

H-D-Asp(OBzl)-OH (30.0 g, 134 mmol) was suspended in 450 mL of anhydrous THF and heated to 50° C. Phosgene (20% in toluene) (100 mL, 100 mmol) was added the amino acid suspension. The amino acid dissolved over the course of approx. 50 min and was left reacting for another 30 min. The solution was concentrated on the rotovap, the white solid redissolved in a toluene/THF mixture (250 mL/50 mL) and the clear solution rotovaped to dryness. The white solid obtained was redissolved into 250 mL of THF, transferred to a beaker, and dry hexanes were added to precipitate the product. The white solid was isolated by filtration and rinsed twice with dry hexanes (2×400 mL) The NCA was isolated by filtration and dried in vacuo. 26.85 g (83.2% yield) of D-Asp(OBzl) NCA was isolated as a white solid. ¹H NMR (d₆-DMSO) δ 9.00 (1H), 7.48-7.25 (5H), 5.13 (2H), 4.69 (1H), 3.09 (1H), 2.92 (1H) ppm.

### Example 25

### Preparation of Poly[DAsp(OBzl)-co-LAsp(OBzl)]-Ac

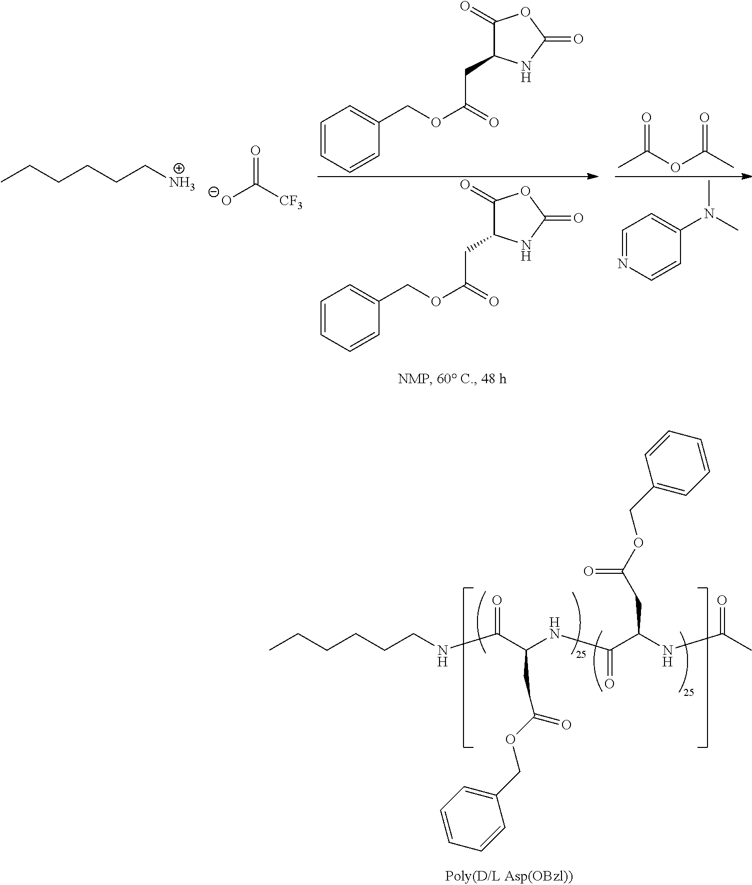

Poly(DLAsp(OBzl)) was synthesized as depicted in Scheme 2. A stock solution of hexylamine/DFA (0.5M in NMP) was prepared. Asp(OBzl) NCA (9 g, 36.1 mmol), DAsp(OBzl) NCA (9 g, 36.1 mmol) were added to a 500 mL 2 neck flask, the flask was evacuated under reduced pressure, and subsequently backfilled with nitrogen gas (repeated twice). Dry N-methylpyrrolidone (NMP) (180 mL) was introduced by cannula, hexylamine/DFA (1.45 mL of stock solution) was syringed in and the solution was heated to 60° C. The reaction mixture was allowed to stir for 4 days at 60° C. under nitrogen gas until disappearance of the starting material by HPLC. The solution was cooled to room temperature and DIPEA (1.0 mL), DMAP (100 mg), and acetic anhydride (1.0 mL) were added. Stirring was continued for 1 hour at room temperature. The polymer was then placed in a 3500 g/mol molecular weight cut-off dialysis bag, dialyzed three times against methanol, three times against deionized water and freeze-dried. A white solid was obtained (7.2 g, 48.6% yield). ¹H NMR (d₆-DMSO) δ 8.61-7.95 (46H), 7.62-6.99 (263H), 5.25-4.79 (108H), 4.76-4.36 (50H), 3.02-2.71 (45H), 2.68-2.51 (39H), 1.86-1.72 (3H), 1.38-1.25 (2H), 1.25-1.08 (5H), 0.83-0.71 (3H) ppm. ¹³C NMR (d₆-DMSO) δ 170.11, 169.90, 135.80, 128.23, 127.83, 127.77, 65.68, 49.78, 35.82, 33.46,33.07, 30.84, 28.72, 25.82, 24.52, 21.92 ppm, PDI=1.1 (DMF/THF GPC), Mn ˜10,000 g/mol (MALDI-TOF MS).

### Example 26

### Synthesis of Poly(DET)

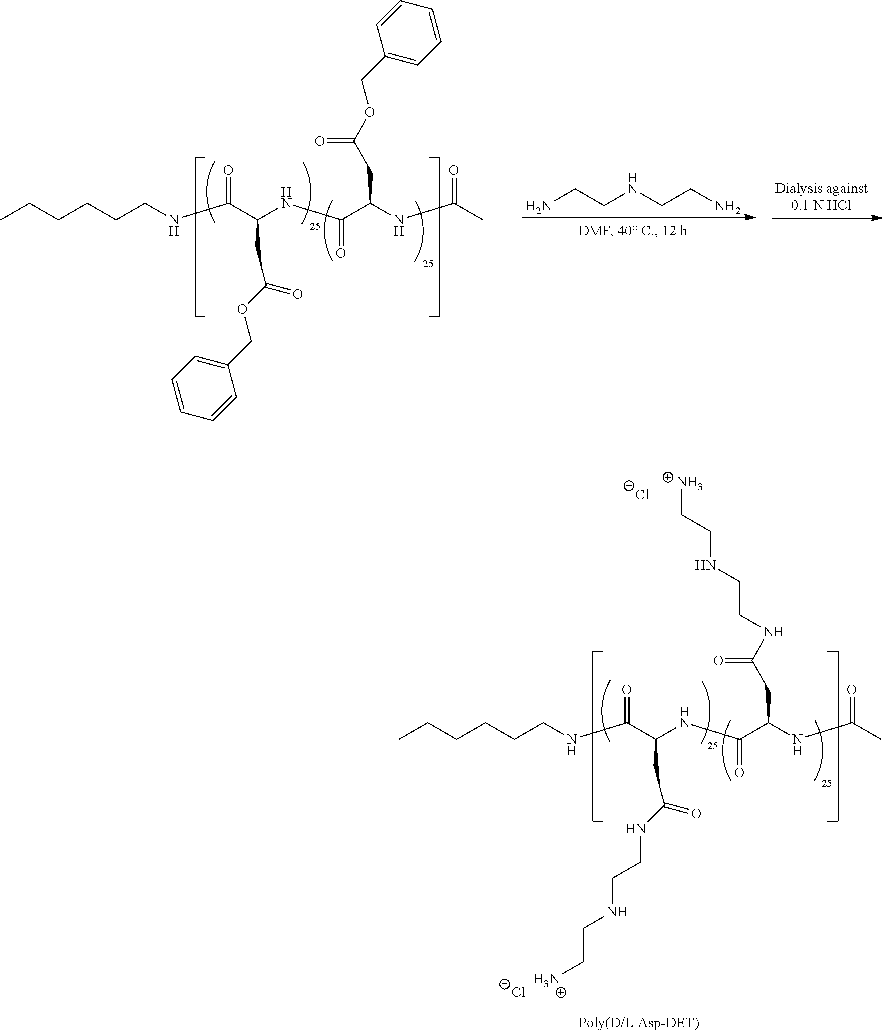

Poly(DLAsp(OBzl)) (2 g, 0.2 mmol) was introduced into an oven-dried two-neck flask and three vacuum/N₂ cycles were done. DET, (26 mL, 242 mmol) and dry DMF (26 mL) were syringed in the reaction flask. The reaction was stirred at 40° C. overnight under inert atmosphere. The reaction solution was then introduced into a 1,000 molecular weight cut-off dialysis bag and dialyzed three times against 0.1 M HCl and three times against deionized water. The solution was filtered through a 0.45 μm filter and the solution was freeze-dried. A highly hygroscopic white fluffy solid was recovered (0.4 g, 17% yield) ¹H NMR (D₂O) δ 4.28-3.97 (23H), 3.76-3.40 (447H), 3.38-3.14 (740H), 3.13-2.99 (254H), 2.99-2.61 (370H), 2.11-1.99 (4H), 1.92 (2H), 1.55-1.44 (2H), 1.35-1.22 (7H), 0.95-0.79 (3H) ppm.

### Example 27

### mPEG-Carboxylic Acid, 5K

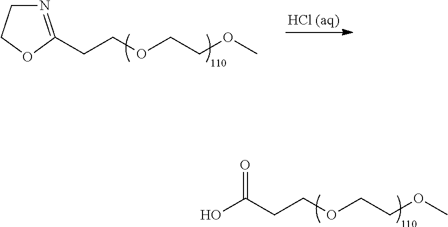

The desired PEG (1 g) was dissolved in 10 mL of 3 N HCl (aq) and stirred at reflux for 4 hours. The solution was cooled then extracted with CHCl₃ (4×300 mL). The combined organic layers dried over MgSO₄, and filtered. The solvent was removed and the resulting liquid was diluted with a minimal amount of methanol and precipitated in to diethyl ether. A white powder was isolated following filtration.

### Example 28

### mPEG-NHS Ester, 5K

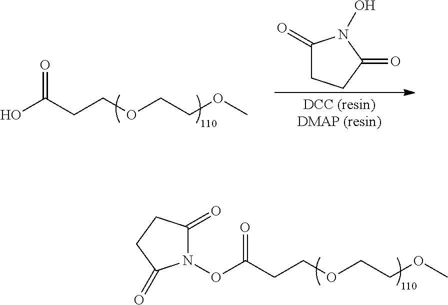

The desired PEG (1 g, 0.15 mmol) was dissolved in dichloromethane (10 mL) then carbodiimide resin (0.58 g, 0.77 mmol) and N-hydroxysuccinimide (0.3 g, 2.6 mmol) were added. The reaction was stirred at room temperature overnight then filtered. The solvent was removed and the resulting liquid was diluted with a minimal amount of methanol and precipitated in to diethyl ether. A white powder was isolated following filtration.

### Example 29

### mPEG-Succinic Acid, 10K

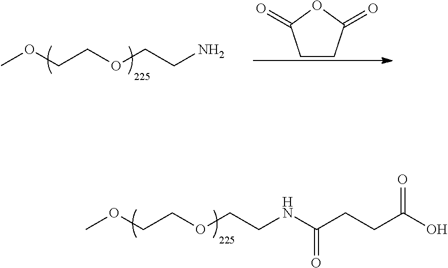

The mPEG-NH2 (1 g, 0.15 mmol) was dissolved in saturated potassium carbonate solution (10 mL) then succinic anhydride (0.83 g, 0.83 mmol). The reaction was stirred at room temperature overnight then extracted with CH₂Cl₂ (4×300 mL). The combined organic layers dried over MgSO₄, and filtered. The solvent was removed and the resulting liquid was diluted with a minimal amount of methanol and precipitated in to diethyl ether. A white powder was isolated following filtration.

### Example 30

### mPEG-NHS Ester, 10K

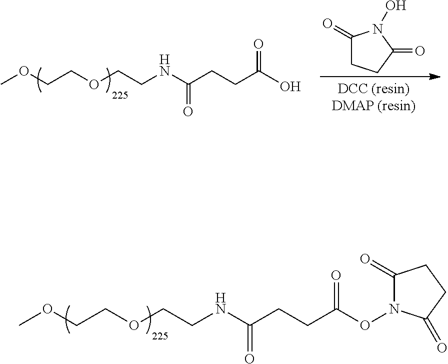

The mPEG-succinic acid (1 g, 0.08 mmol) was dissolved in dichloromethane (15 mL) then carbodiimide resin (0.6 g, 0.77 mmol) and N-hydroxysuccinimide (0.2 g, 1.7 mmol) were added. The reaction was stirred at room temperature overnight then filtered. The solvent was removed and the resulting liquid was diluted with a minimal amount of methanol and precipitated in to diethyl ether. A white powder was isolated following filtration.

### Example 31

### N3-PEG-Succinic Acid, 12K

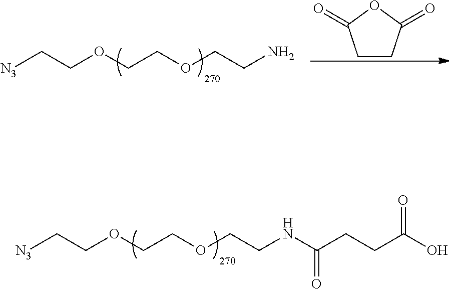

The N3-PEG-NH2 (1 g, 0.15 mmol) was dissolved in saturated potassium carbonate solution (10 mL) then succinic anhydride (0.83 g, 0.83 mmol). The reaction was stirred at room temperature overnight then extracted with CH₂Cl₂ (4×300 mL). The combined organic layers dried over MgSO₄, and filtered. The solvent was removed and the resulting liquid was diluted with a minimal amount of methanol and precipitated in to diethyl ether. A white powder was isolated following filtration.

### Example 32

### N3-PEG-NHS Ester, 12K

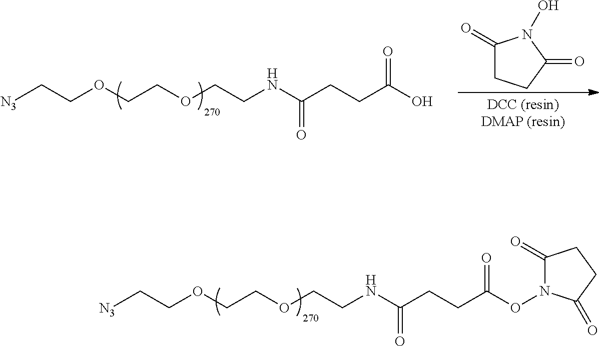

The N3-PEG-succinic acid (1 g, 0.08 mmol) was dissolved in dichloromethane (15 mL) then carbodiimide resin (0.6 g, 0.77 mmol) and N-hydroxysuccinimide (0.2 g, 1.7 mmol) were added. The reaction was stirred at room temperature overnight then filtered. The solvent was removed and the resulting liquid was diluted with a minimal amount of methanol and precipitated in to diethyl ether. A white powder was isolated following filtration.

### Example 33

### mPEG-Oxanorbornene, 5K

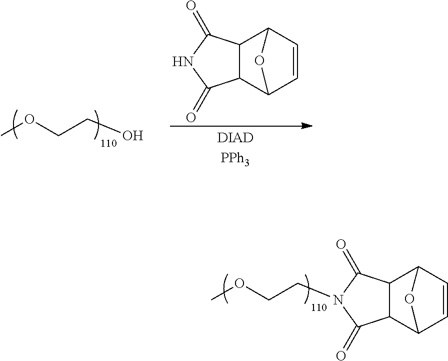

The mPEG (2 g, 0.4) was dissolved in dichloromethane (25 mL). Triphenylphosphine (0.42 g, 1.6 mmol) followed by the oxanorbornene (0.26 g, 1.6 mmol) then DIAD (0.24 mL, 1.2 mmol) was added to the solution then stirred for 8 hours. The solvent was removed and the viscous liquid containing the desired polymer was loaded onto 100 g silica gel which was rinsed with 3% MeOH in CHCl₃ (1 L) followed by 10% MeOH in CHCl₃ (1 L) which contained the polymer product. The solvent was removed and the resulting liquid was diluted with a minimal amount of methanol and precipitated into diethyl ether. A white powder was isolated following filtration.

### Example 34

### mPEG-Maleimide, 5K

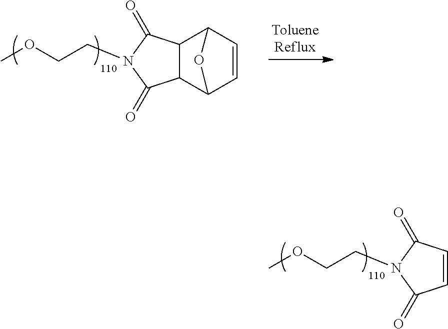

The mPEG-oxanorbornene (2 g) was dissolved in toluene (20 mL) and refluxed for 4 hours. After allowing the solution to cool, the polymer was precipitated in to diethyl ether. A white powder was isolated following filtration.

### Example 35

### Nucleic acid/polymer Complexation and PEGylation with Maleimide Chemistry

Polymer (Example 26) were prepared at a N:P ratio of 50 in H₂O, based on a final amount of 20 μg Luciferase plasmid DNA (See FIG. 13 for schematic). The polymers were filter sterilized using a 0.22 μm PES filter and then complexed with 100 uL plasmid DNA at N:P ratio 50, in a final volume of 200 μL, for 30 min at room temperature. 0.5 uL of 3.23M KOH was added to the polyplex solution to increase the pH to between 7-8. Fifty uL of 5K or 10K maleimide PEG (From Example 34, 60 mg/mL stock solutions) was added to polyplexes and incubated at 37 C with shaking for three hours. Post-PEG polyplexes were resolved on 1% agarose/ethidium bromide gel in 1×TAE Buffer for 30 min at 200V, FIG. 1 and FIG. 4. The agarose/ethidium bromide gel was stained with Coomassie blue for 30 min and destained overnight using H2O.

### Example 36

### Nucleic Acid/Polymer Complexation and Polyplex Post-PEG Procedure with NHS Ester Chemistry

Poly(d/l Asp-DET)/DNA polyplexes were prepare by adding equal volumes of Poly(d/l Asp-DET) (From Example 26) solution (dissolved in dH₂O) and plasmid DNA solution (200 μg/mL in dH₂O) at N:P 10 ratio. (See FIG. 13 for schematic) Polymer was added to DNA solution, for a final volume of 200 μL, and incubated at room temperature for at least 30 minutes to allow polyplex formation. Fifty uL of 12 k succinimide-PEG (From Example 32, 60 mg/mL stock solution in H₂O) was added to polyplexes and incubated at room temperature with shaking for three hours to create PEG-polyplexes. Fifty uL of dH₂O was added to non-PEG polyplexes to achieve equal volumes for all samples.

### Example 37

### Gel Retardation Experiments

Polymers were prepared at an N:P ratio of 50 in H2O, based on a final amount of 20 μg Luciferase plasmid DNA (pGL4; Promega, Madison, Wis.). The polymers were filter sterilized using a 0.22 μm PES filter and then complexed with 100 uL plasmid DNA at N:P ratios between 2.5 and 50, in a final volume of 200 μL, for 30 min at room temperature. Gel loading dye was added to each polymer/DNA complex and samples run on a 1% agarose/ethidium bromide gel in 1×TAE Buffer for 30 min at 200V, FIGS. 1 and 4. FIG. 1 shows the agarose/ethidium bromide gel was post-stained with Coomassie blue for 30 min and then destained overnight using H2O. DNA retardation was observed in both DNA/polymers samples at N:P ratios of 2.5. Wells containing intact naked DNA served as controls. Po; polymer only, C; complex, 1 kb; One kb DNA ladder. Agarose/ethidium bromide gels were post-stained with Coomassie blue. Free polymer was detected in all samples with an overall decrease in the amount of free polymer in complexed samples. FIG. 4 shows the results when twenty μg of pGL4 plasmid DNA was complexed with Poly(D/L Asp-DET) at N:P 50 for 30 min at room temperature. Polyplexes were the pH adjusted to 7-8 and then incubated with 5 k or 10 k PEG for three hours at 37 C. Samples were then resolved on a 1% agarose/ethidium bromide gel. DNA retardation was observed in all polyplex samples. Wells containing intact naked DNA served as controls. Po; polymer only, C; complex, 1 kb; One kb DNA ladder. Agarose/ethidium bromide gels were post-stained with Coomassie blue. The degree of PEGylation of free polymer could be determined by Coomassie blue staining of gels.

### Example 38

### Polymer/DNA Complex Size Analysis

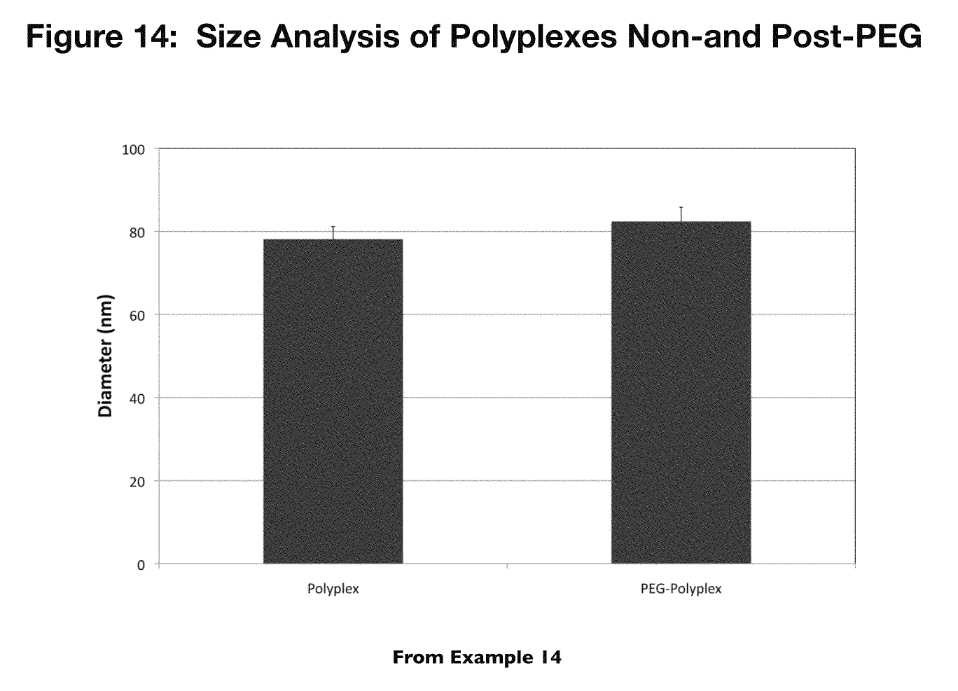

Non-and PEG polyplexes were prepared as described above. Dynamic Light Scattering analysis was performed using a DynaPro Dynamic Light Scattering Plate Reader (Wyatt Technology Corporation, Santa Barbara, Calif.). One hundred and twenty μL of each sample was loaded into a 96 well plate and sizes determined every hour with ten 30 sec acquisitions at 37° C., FIGS. 2, 5, and 14. For FIG. 2A: Dynamic light scattering analysis of polyplex size for the D/L polymer between N:P ratios of 2.5 and 50 ranged from ˜170 to 53 nm. FIG. 2B: Time course experiments at 37° C. demonstrated no change in polyplex size for N:P ratios greater than 5. For FIG. 5A: Dynamic light scattering analysis of pre- and post-PEG polyplexes at N:P 50 (from Example 35) FIG. 5B: Time course experiments at 37 C demonstrated no change in polyplex sizes for Polyplex alone and 5 k PEG-polyplexes while 10K PEG-Polyplexes increase in size over time. For FIG. 14: Dynamic light scattering analysis of non- and post-PEG polyplexes at N:P 10, prepared according to Example 36.

### Example 39

### Polymer Titration Experiments

Three mg of polymer was diluted in a 10 mL final volume of 150 mM NaCl. The polymer solution (from Example 26) was titrated with 1N HCl and plotted as a function of pH, FIG. 3. Asp-DET polymers exhibit buffering capacity within the critical pH buffering area of the curve corresponding to the transition from the endosome to the lysosome (pH 5-7).

### Example 40

### Transmission Electron Microscopy of Polyplexes

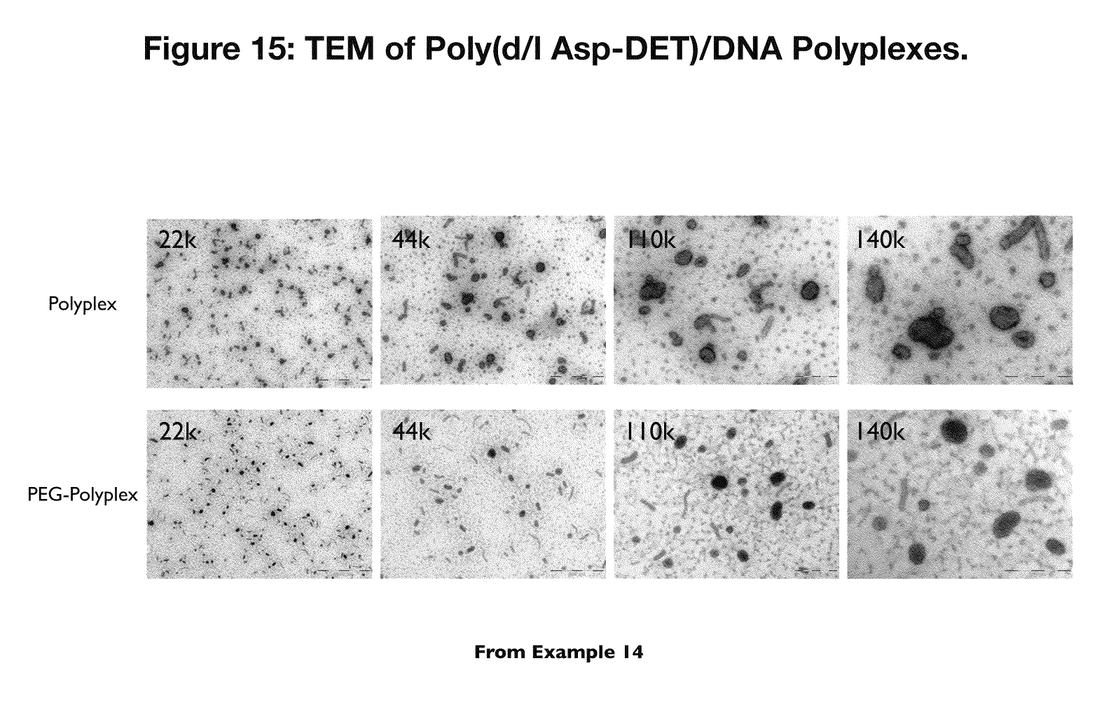

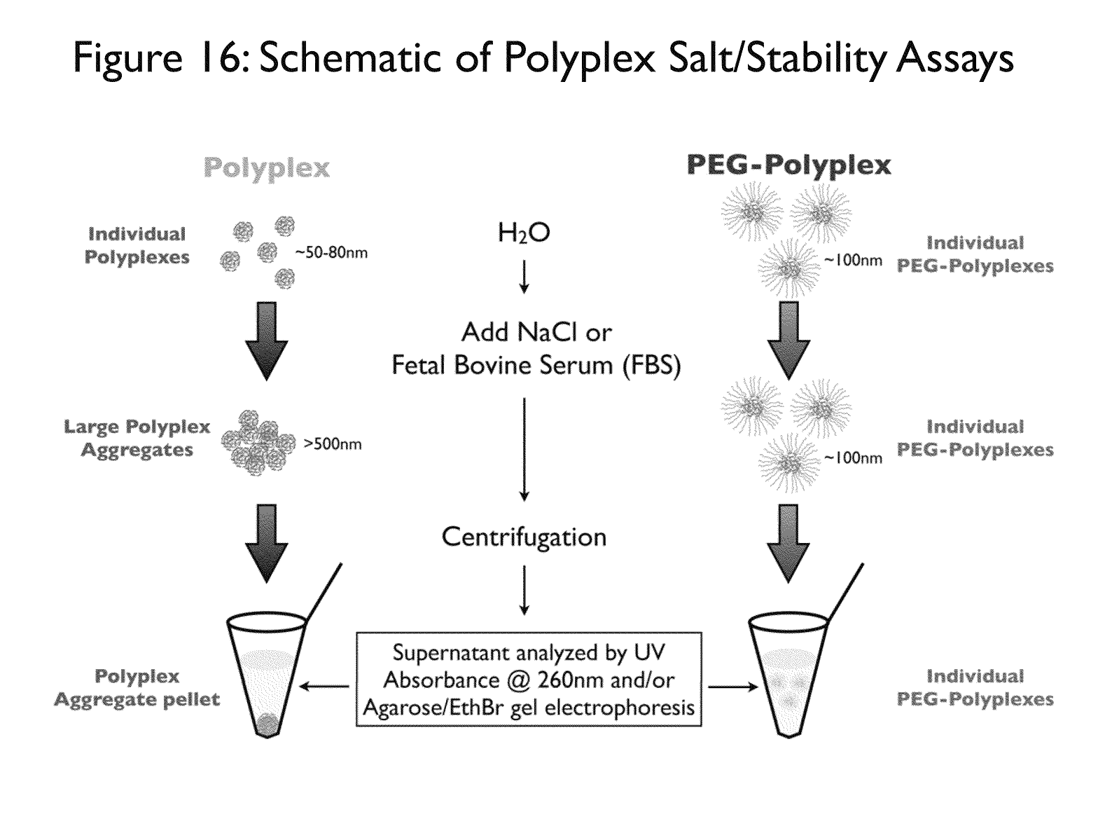

Non- and PEG polyplexes were prepared as in Example 35. Five uL of each sample was spotted onto formvar grids for 1-5 min, washed with H2O, incubated with 5% uranyl acetate for 1 min and washed again in H2O. Images were taken using a Morgagni 268D electron microscope, FIG. 6. FIG. 15 shows results when polyplexes are prepared according to Example 36. Poly(d/l Asp-DET) polymers interacted with plasmid DNA to form uniform structures which were less than 150 nm in size. Post-PEG polyplexes showed similar morphology and were also smaller than 150 nm.

### Example 41

### Erythrocyte Aggregation Assay

Non- and PEG polyplexes were prepared as described above as in Example 35. Thirty μL of each sample was spiked with 5M NaCl for final 150 mM concentration. Samples were then incubated with erythrocytes (60 uL) in 96 well plates and incubated at 37 C for 1 hour. Results shown in FIG. 7 demonstrate that P(Asp-DET)/DNA polyplexes incubated with erythrocytes resulted in extensive cell lysis. In contrast, incubation with post-PEG polyplexes resulted in no change to erythrocytes, similar to the PBS incubated control.

### Example 42

### Transfections and Plasmid Visualization Experiments

HCT-116 colon cancer cells, obtained from ATCC, were maintained in McCoy's media supplemented with 10% FBS, 2 mM L-glutamine, and 100 units/mL penicillin/streptomycin. Twenty-five thousand HCT-116 cells, in a total volume of 100 μL McCoy's media, were seeded in each well of a 96-well format plate the day before transfection. On the day of transfection, non- and PEG polyplexes were prepared as described above. HCT-116 cells were transfected with either an EGFP plasmid (pZs-Green; Clontech, Mountain View, Calif.) or pGL4-luciferase plasmid, (Promega, Madison, Wis.). Transfection complexes (2.5 μL) were added to the cells and incubated at 37° C. After 24 hours incubation, the cells were either visualized with an Olympus IX71 microscope or luciferase activity was determined using a standard luciferase assay kit (Promega). Protein quantitation was also determined using the Bradford Assay (Bio-Rad Labs, Hercules, Calif.). Experiments with the commercially available transfection reagents jetPEI (Polyplus Transfection Inc, New York, N.Y.) and Superfect (Qiagen, Valencia, Calif.) were also performed using the manufacturers' recommended protocols. Furthermore, transfection experiments for each polymer and commercial transfection reagent was performed in triplicate, and the luciferase activity was normalized to the quantity of protein in each well. FIG. 8 shows a comparison of luciferase transfection efficiencies for P[Asp(DET)] versus commercial reagents. FIG. 9 demonstrates transfection of EGFP between P[Asp(DET)] and 5 k and 10K PEG P[Asp(DET)] polymers. For plasmid visulalization experiments, EGFP plasmid (pZs-Green; Clontech, Mountain View, Calif.) was fluorescently labeled with 5-carboxy-X-rhodamine using the Label IT®Tracker™ Kit (Minis, Madison, Wis.). Twenty-four hours after transfection, cells were visualized with an Olympus IX71 microscope and results shown in FIG. 10. Cells were observed by phase contrast (left panel) or fluorescent microscopy (middle panels). Cells expressing pZs-Green GFP protein (green) also contained various amounts of labeled DNA (red) in both the nucleus and cytoplasm. Merged images appear in the right panels. ×40 magnification.

### Example 43

### In Vivo Polymer/DNA Delivery Experiments

On the day of experiment, 250 uL of PEG polyplexes containing pGL4-luciferase plasmids were prepared as described above in Example 35. Twenty% glucose was added to samples for a final 5% glucose concentration. The entire glucose/PEG/polyplex sample was administered by tail vein IV administration to tumor bearing nude mice, FIG. 11. At various time points, mice were anesthetized and imaged using the IVIS Spectrum system (Caliper Life Sciences, Hopkinton, Mass.). At the completion of the experiment, mice were anesthetized, sacrificed by cervical dislocation and various tissues collected. DNA and RNA samples were extracted from tissue samples using the Qiagen AllPrep DNA/RNA Kit. RT-PCR and PCR was performed using pGL4 specific primers, FIG. 11A shows IVIS images of mice 72 hours after IV injection. HCT-116 tumors are circled red and lymph nodes are circled purple. FIG. 11B shows PCR and FIG. 11C shows RT-PCR results of tumor and lymph node tissues. Plasmid DNA accumulation was demonstrated in both tumors and lymph nodes while gene expression was observed in lymph nodes. FIG. 12 shows PCR results of various organs from treated nude mice detected high plasmid DNA levels in liver and kidney and moderate levels in spleen.

### Example 44

### Salt Addition and Centrifugation Studies

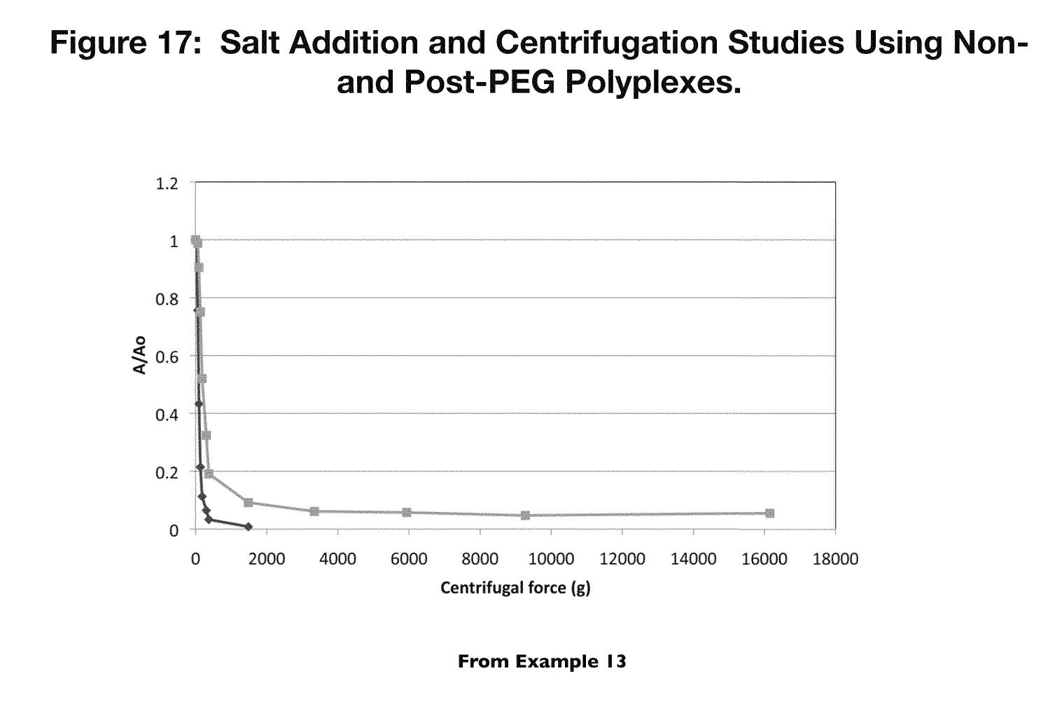

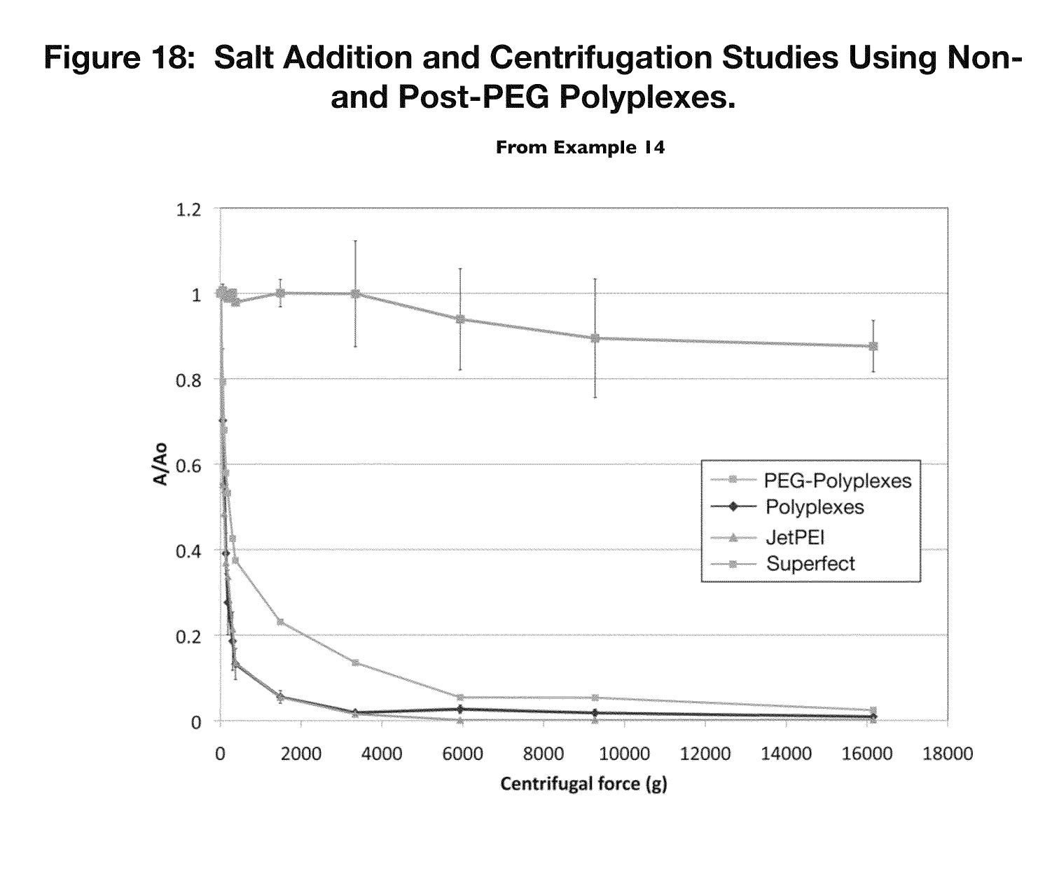

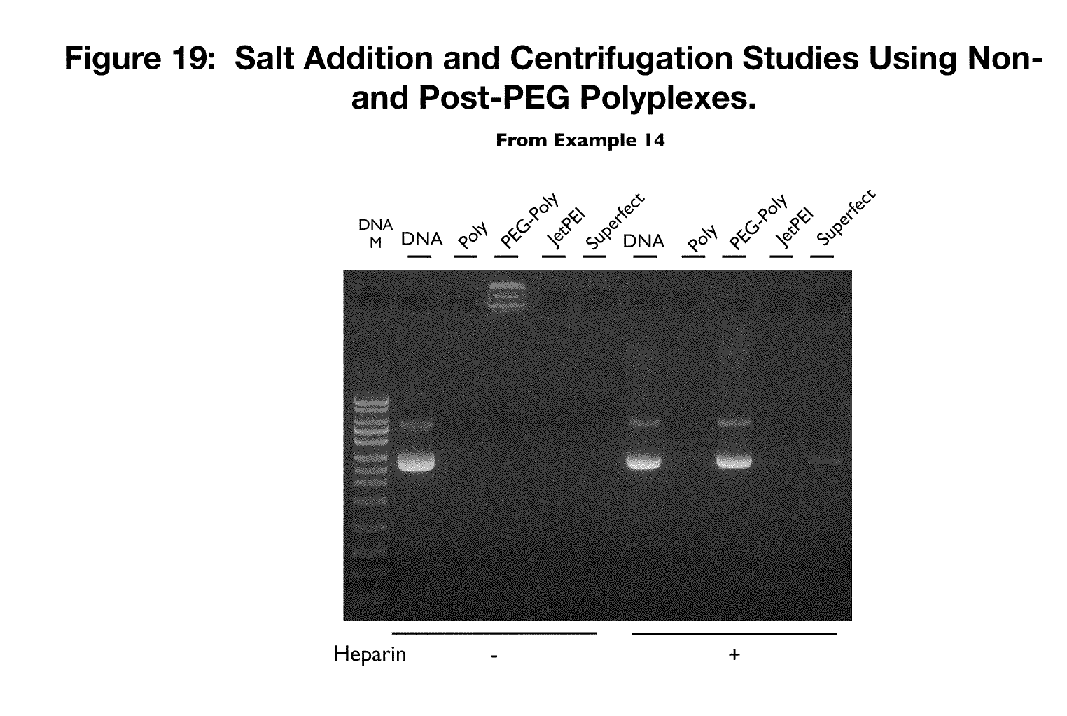

Non- and PEG-polyplex samples (as described above in Examples 35 and 36), along with complexes made with JetPEI and Superfect, were spiked with 5M NaCl for a final 150 mM concentration. Experiments using JetPEI (Polyplus-transfection Inc. New York, N.Y.) and Superfect (Qiagen, Valencia, Calif.) were also performed using the manufacturers' recommended protocols. Samples were incubated, initial UV absorbance at 260 nm measured, and samples centrifuged at intervals of increasing g forces for 1 minute. After each centrifugation step, supernatant UV absorbance was determined at 260 nm. A/Ao ratios were calculated for each centrifugation step. Ao; initial sample absorbance value at 260 nm, A; absorbance of sample supernatant after each centrifugation. A schematic representing this experiment is shown in FIG. 16. Data are the average ±SD (n=2). FIG. 17 shows the results of this experiment with polyplexes and PEG-polyplexes made according to Example 35. FIG. 18 shows the results for polyplexes and PEG-polyplexes made according to Example 36. It is important to note that the PEG-polyplexes prepared according to Example 36 remain in solution following centrifugation and are therefore stable in solution and have not aggregated following salt addition. After the final centrifugation, supernatant samples were resolved on a 1% agarose/ethidium bromide gel, shown in FIG. 19. Heparin was added to duplicate samples to dissociate DNA from polymers. Poly; Poly(d/l Asp-DET)/DNA polyplex, DNA M; 1 kb DNA ladder. It is also important to note that the PEG-polyplexes prepared according to Example 36 still contain intact DNA following the salt-induced aggregation study, and the intact DNA is in the supernatant following centrifugation.

### Example 45

### Serum Addition and Centrifugation Studies

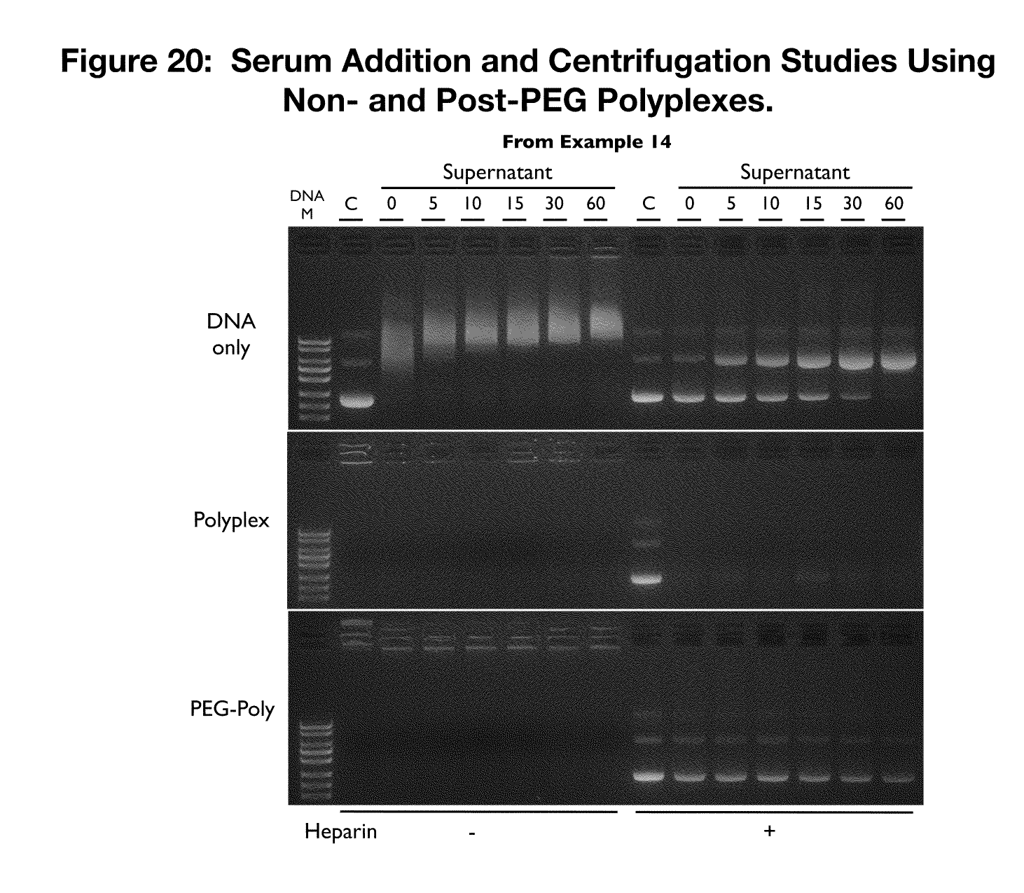

DNA, polyplexes, and PEG-polyplexes prepared (according to Example 36) were incubated with an equal volume of FBS at 37° C. for up to 1 hour. Samples were then centrifuged and the supernatant was analyzed by agarose gel electrophoresis. Equal volumes of supernatant samples were loaded per well. Heparin was added to duplicate samples to dissociate DNA from polymers. Results are shown in FIG. 20. C; Samples in H₂O, Supernatant; Sample supernatant following serum incubation (min) and centrifugation. Poly; Poly(d/l Asp-DET)/DNA polyplex, DNA M; 1 kb DNA ladder. These results indicate that DNA only rapidly associates with plasma proteins, but is quickly degraded. The polyplexes (non-PEG version) aggregates quickly and is spun out of solution. However, the PEG-Polyplex remains in solution following addition of serum, and the DNA within the polyplex remains intact.

### Example 46

### Luciferase Expression of Cells Transiently Transfected with Poly(d/l Asp-DET) Polymers

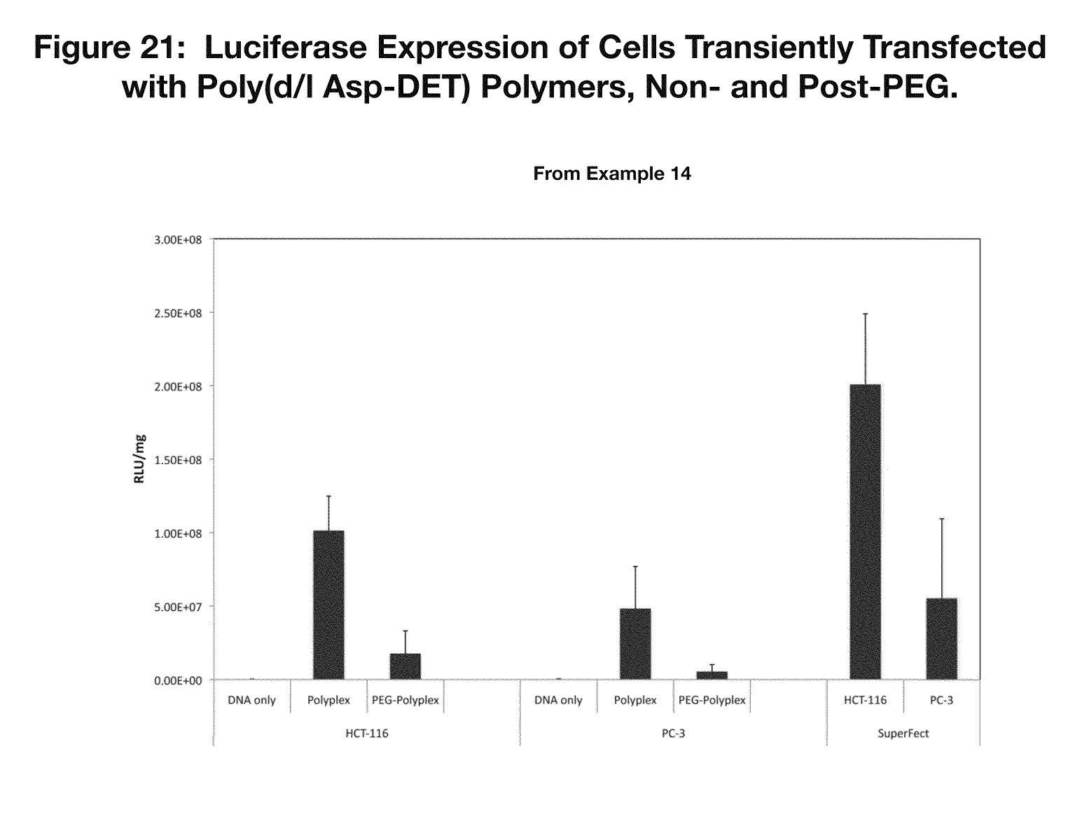

Human HCT-116 colon cancer cells and PC-3 prostate cancer cells were purchased from ATCC (Rockville, Md.). HCT-116 cells were maintained in McCoy's media supplemented with 10% FBS, 2 mM L-glutamine, and 100 units/mL penicillin/streptomycin, while PC-3 cells were maintained in RPMI 1640, 10% FBS, 2 mM L-glutamine, and 100 units/mL penicillin/streptomycin. Media and supplements were purchased from Cellgro (Mediatech Inc. Manassas, Va.). All cells were cultured at 37° C. in a 5% CO₂ humidified atmosphere. The day before transfection, 25000 HCT-116 cells and 10000 PC-3 cells were plated in 96-well culture plates, in a total volume of 100 μL media. On the day of transfection, polyplexes were prepared as described above in Example 36 with pGL4-luciferase plasmid. Transfection complex (2.5 uL for polyplex and 3.12 uL for PEG-polyplex) was added to the cells and incubated at 37° C. Twenty-four hours after incubation, luciferase activity was determined using a luciferase assay kit (Promega). Protein quantity was determined using the Bradford Assay (Bio-Rad Labs, Hercules, Calif.). Experiments with Superfect (Qiagen, Valencia, Calif.) were also performed using the manufacturers' recommended protocols. Transfection experiments for polyplexes and commercial transfection reagent was performed in triplicate, and the luciferase activity was normalized to the quantity of protein in each well. FIG. 21 demonstrates that the PEG-polyplexes transfect PC-3 and HCT-116 cells.

### Example 46a

### Poly(D/L Asp-DET) Polymer Titration

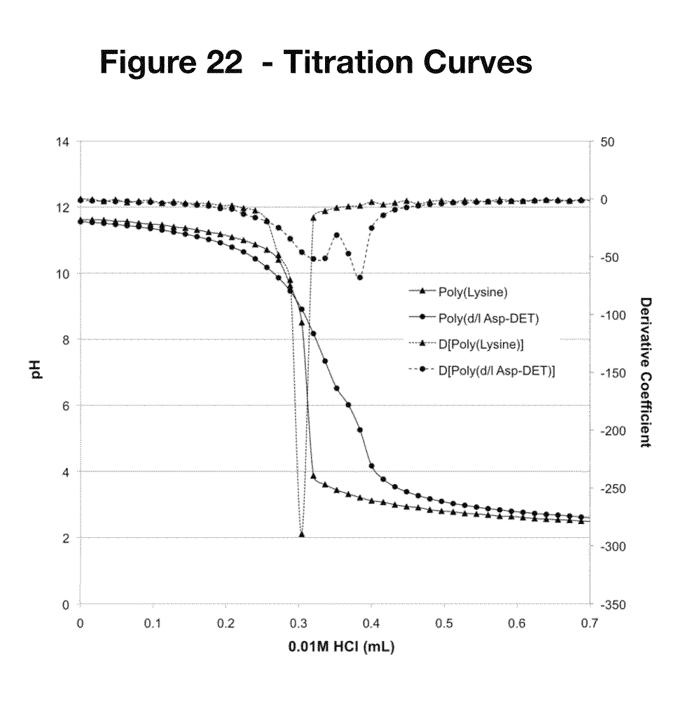

Poly(D/L Asp-DET) was dissolved to a concentration of 77 μM amines in 10 mL of 150 mM NaCl and titrated with 0.01N HCl. pH measurements were performed at 25° C. with a 702 SM Titrino (Metrohm AG, Switzerland). Poly-L-Lysine (MW 150000-300000, Sigma) was used as a control. The second derivative curves were determined from the obtained titration curves, FIG. 22.

### Example 47

### Formulation of Polymer/Nucleic Acid Polyplexes

Poly(D/L Asp-DET)/DNA polyplexes were prepared by adding equal volumes of Poly(D/L Asp-DET) solution (dissolved in dH₂O) and plasmid DNA solution (200 μg/mL in dH₂O) at the appropriate N:P ratio. Polymer was added to DNA solution, for a final volume of 200 μL, and incubated at room temperature for at least 30 min to allow polyplex formation. PEG-polyplexes were formed by incubating 200 μL of Poly(D/L Asp-DET)/DNA N:P 10 polyplexes with 50 μL of Azide-12 k PEG-NHS (60 mg/mL in dH₂O, refs) for 3 hr with shaking at room temperature. Un-reacted PEG was removed by ultrafiltration using a Vivaspin 500 100,000 MWCO filters (Sartorius Stedim Biotech GmbH, Germany), and PEG-polyplexes were diluted with dH₂O to a final volume of 200 μL to achieve equal volumes for all samples.

### Example 48

### Gel Retardation Experiments and Ethidium Bromide Exclusion Assays

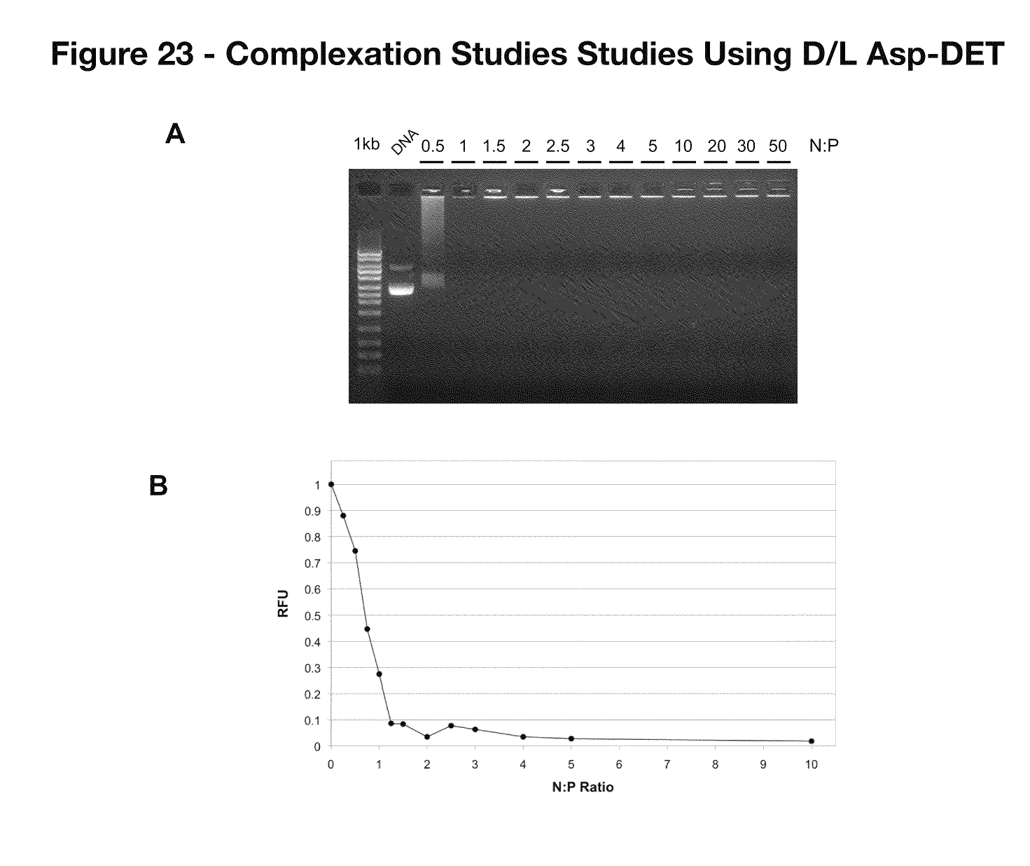

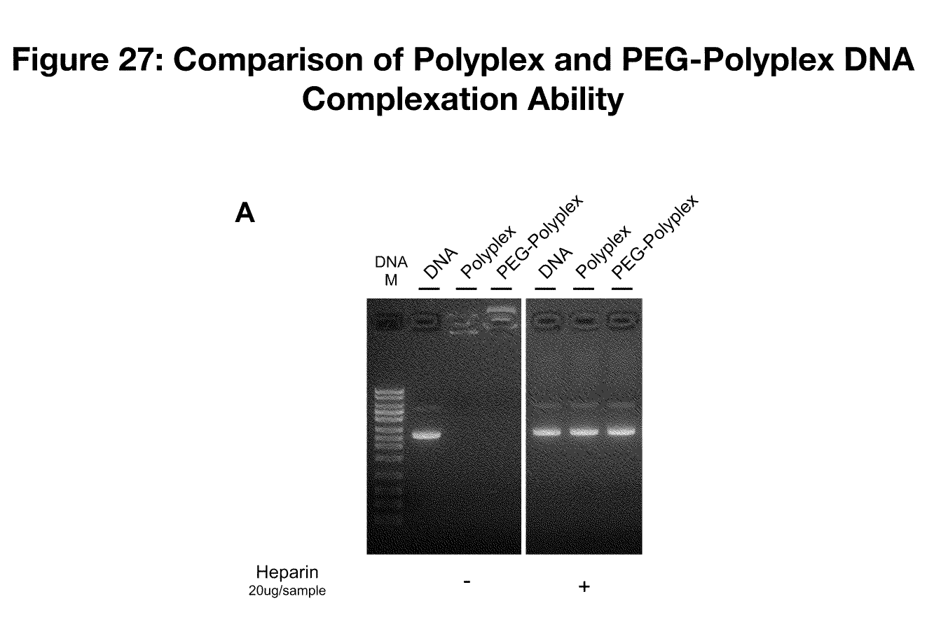

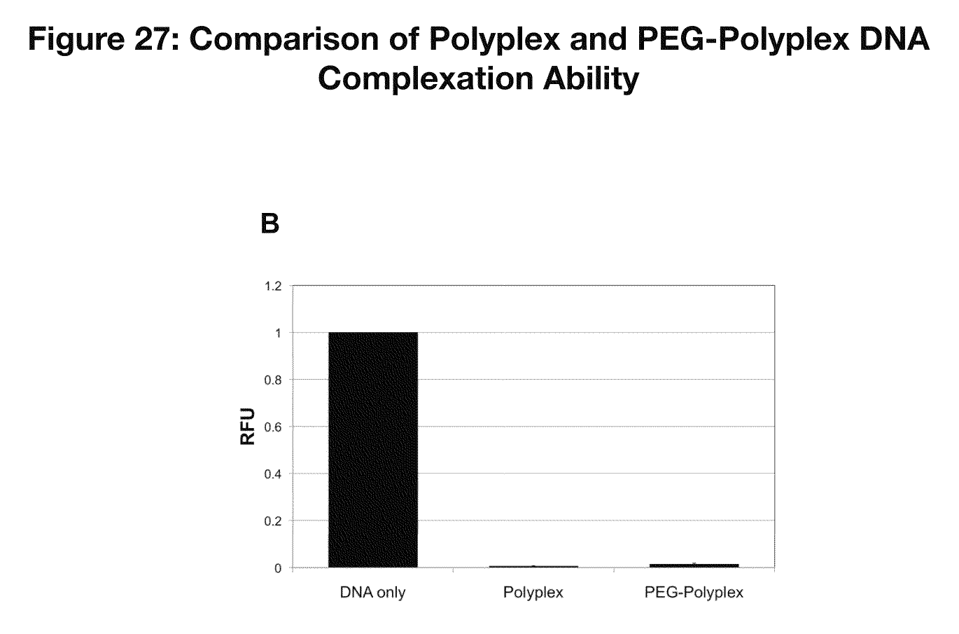

Polyplexes containing Luciferase plasmid DNA (pGL4; Promega, Madison, Wis.) were prepared (as described in Example 47) at various N:P ratios. Five μL of each formulation was run on a 1% agarose gel and visualized by ethidium bromide staining, FIG. 23A and 27A. For ethidium bromide exclusion assays, DNA only (100 μg/mL in H₂O) and polyplex solutions were diluted 1:4 with dH₂O to a final volume of 50 μL. Fifty μL of ethidium bromide (2 ug/mL in H2O), was added to the solutions, mixed and incubated at room temperature for 20 min. The fluorescence intensity of triplicate samples was measured at λ=580 nm (excitation at λ=540) with a spectrofluorometer (FLUORstar OPTIMA, BMG Labtech Inc.). Relative Fluorescence Units (RFU) were calculated using: RFU=(Flₛₐₘₚₗₑ-Fl₀)/(Fl_{DNA}-Fl₀), where Flₛₐₘₚₗₑ, Fl₀ and Fl_{DNA} represent the fluorescence intensity of the samples, background and free plasmid DNA, respectively, FIG. 23B and 27B.

### Example 48a

### Dynamic Light Scattering (DLS) and Zeta-Potential Measurements

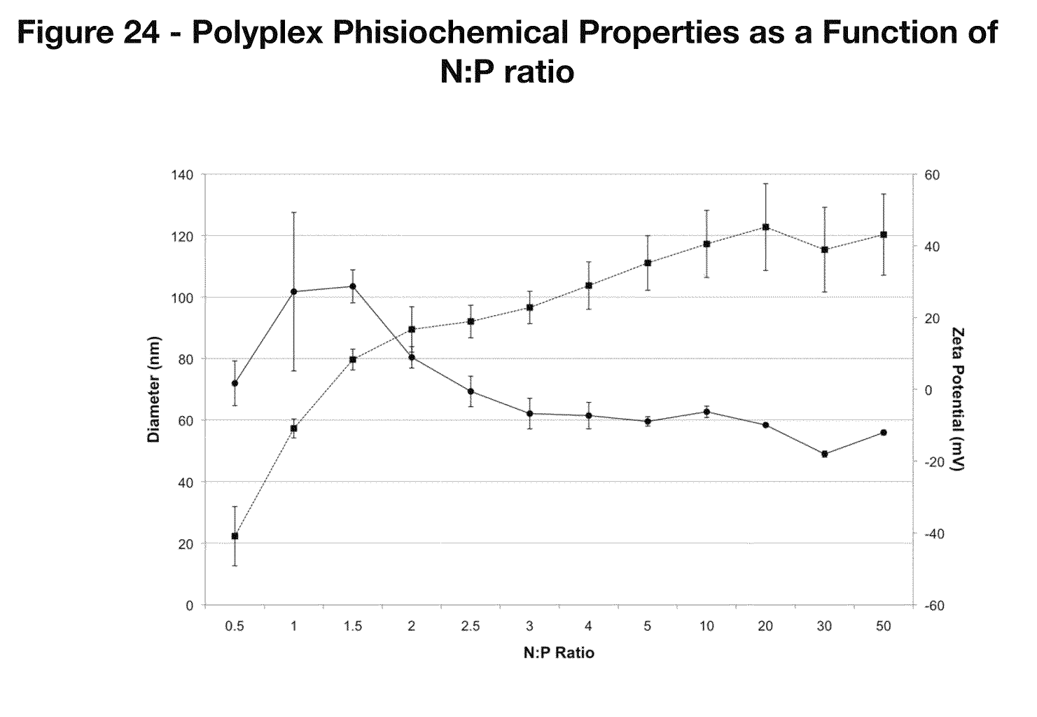

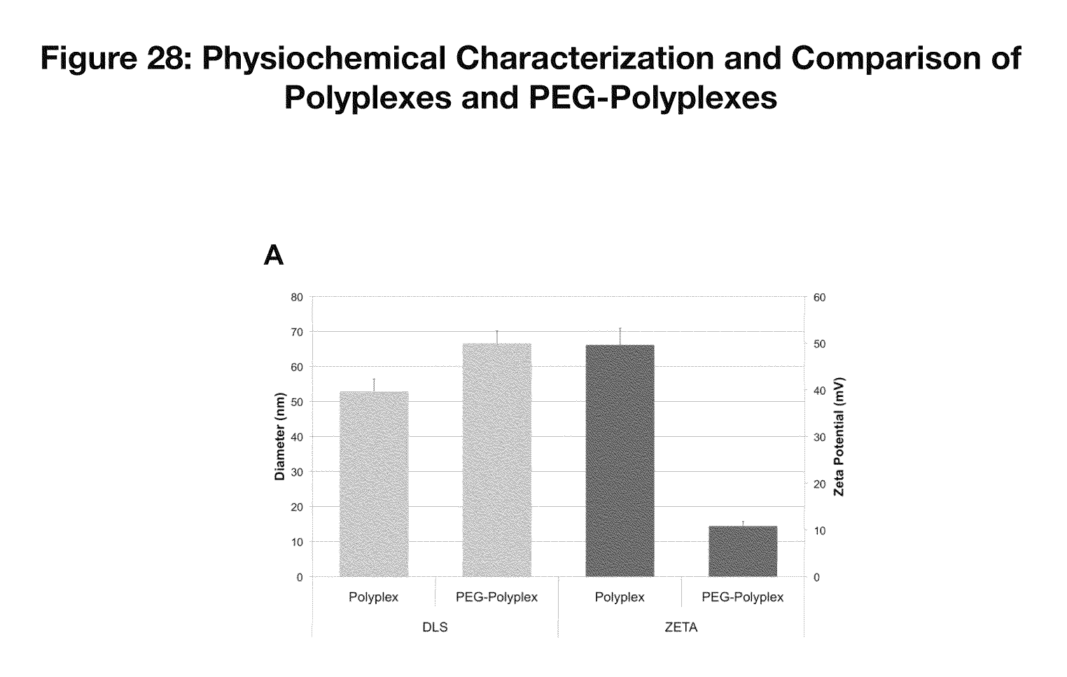

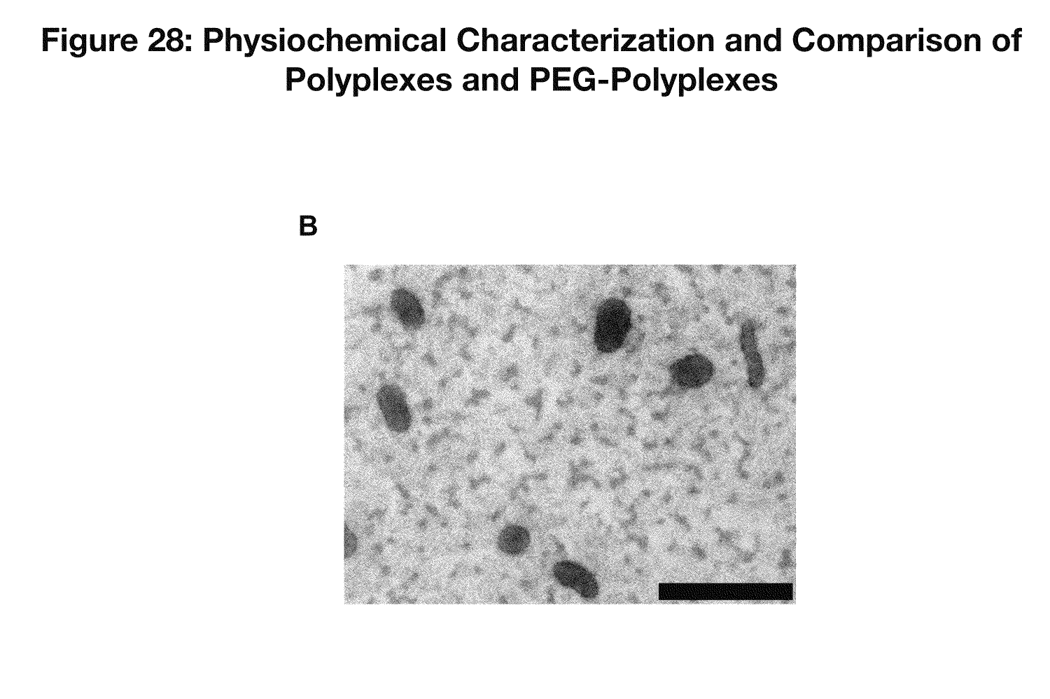

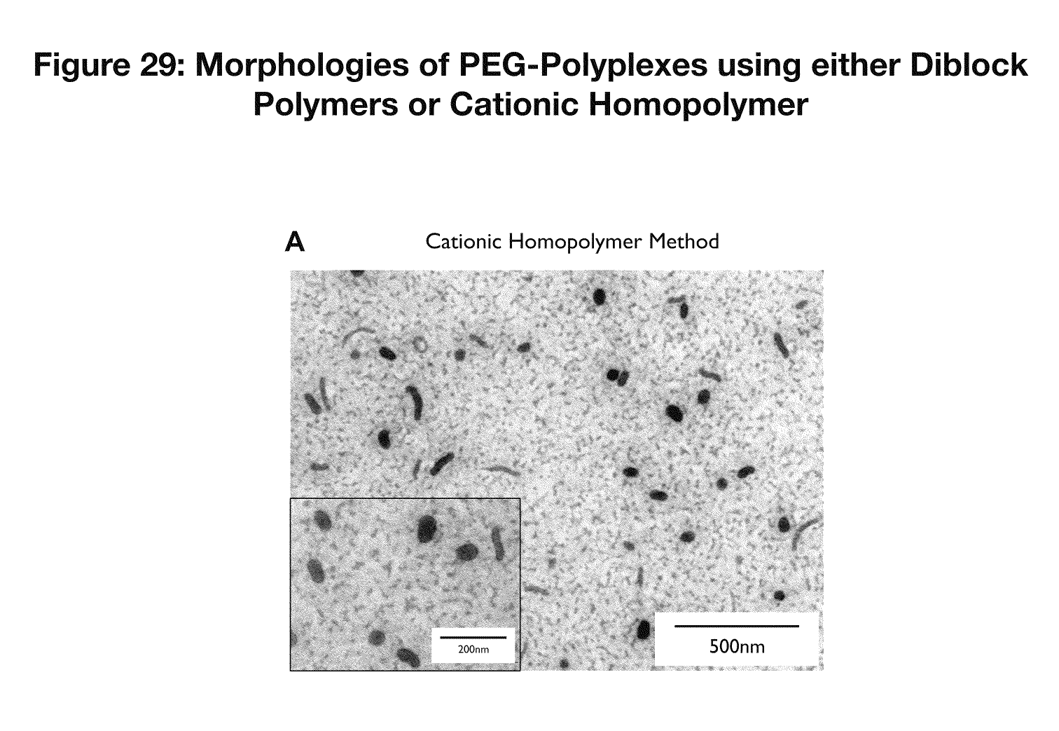

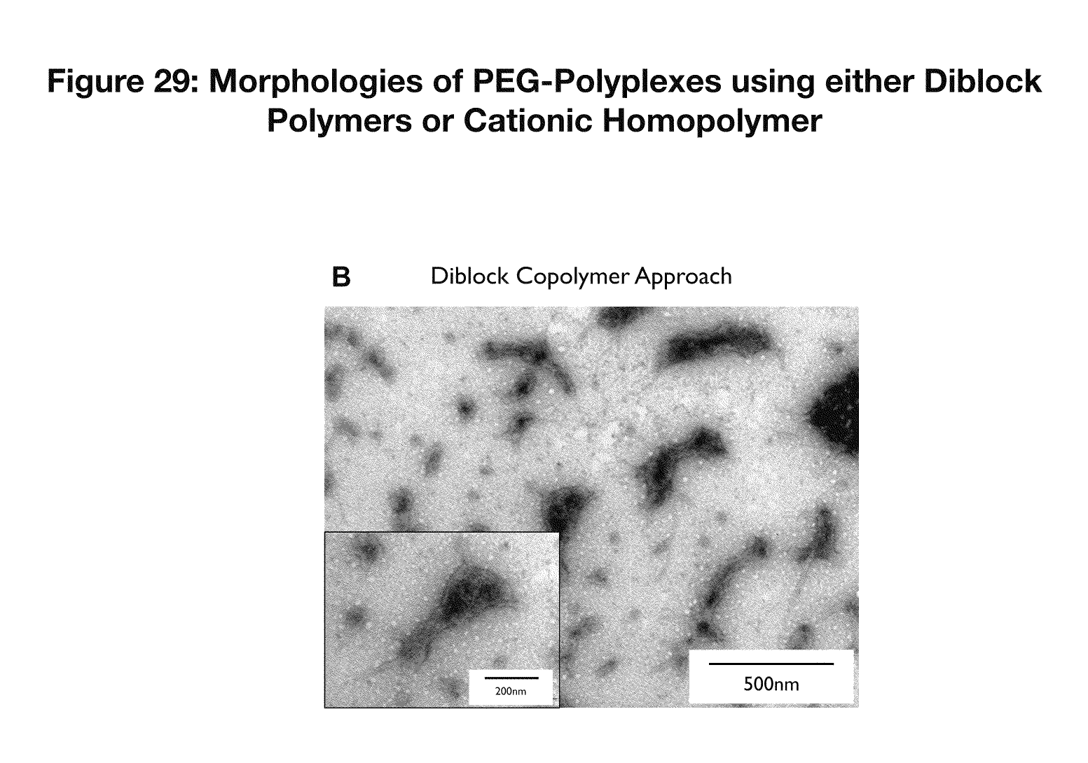

Polyplex sizes were measured using a DynaPro Dynamic Light Scattering Plate Reader (Wyatt Technology Corporation, Santa Barbara, Calif.), and determined every hr for eight hr with ten 30 sec acquisitions at 37° C. Zeta-potential measurements were determined using a Zetasizer Nano instrument (Malvern Instruments Ltd, UK), and represent the average of three runs at 25° C., FIGS. 24 and 28A.

### Example 49

### DNAse Protection Assay

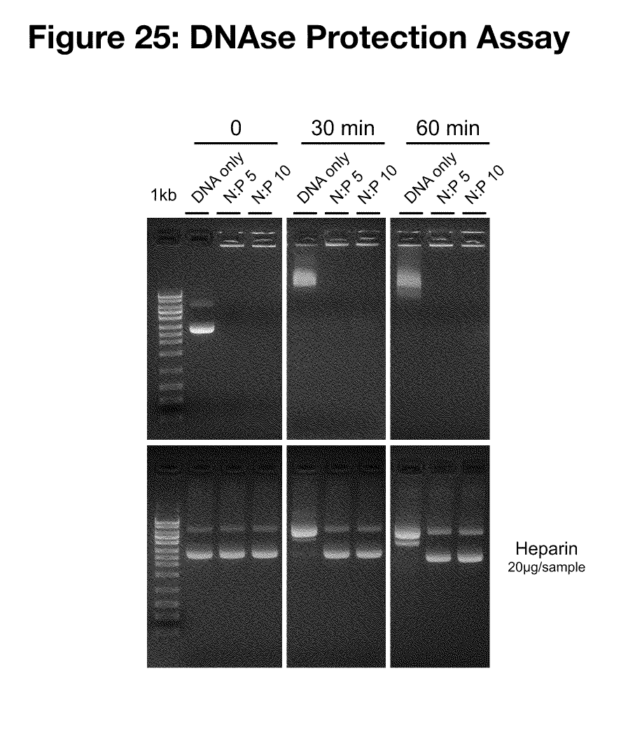

Five μL of each polyplex sample was incubated with 5 μL fetal bovine serum (FBS, Cellgro, Mediatech Inc. Manassas, Va.) at 37° C. for up to 1 hr. Five μL of heparin (2 mg/mL in dH2O) was added to each sample and incubated at room temperature for 10 min to displace polymers from DNA. Samples were electrophoresed in a 1% agarose gel as described for the gel retardation experiment (Example 48), FIG. 25.

### Example 50

### Flow Cytometry Cellular Uptake Experiments

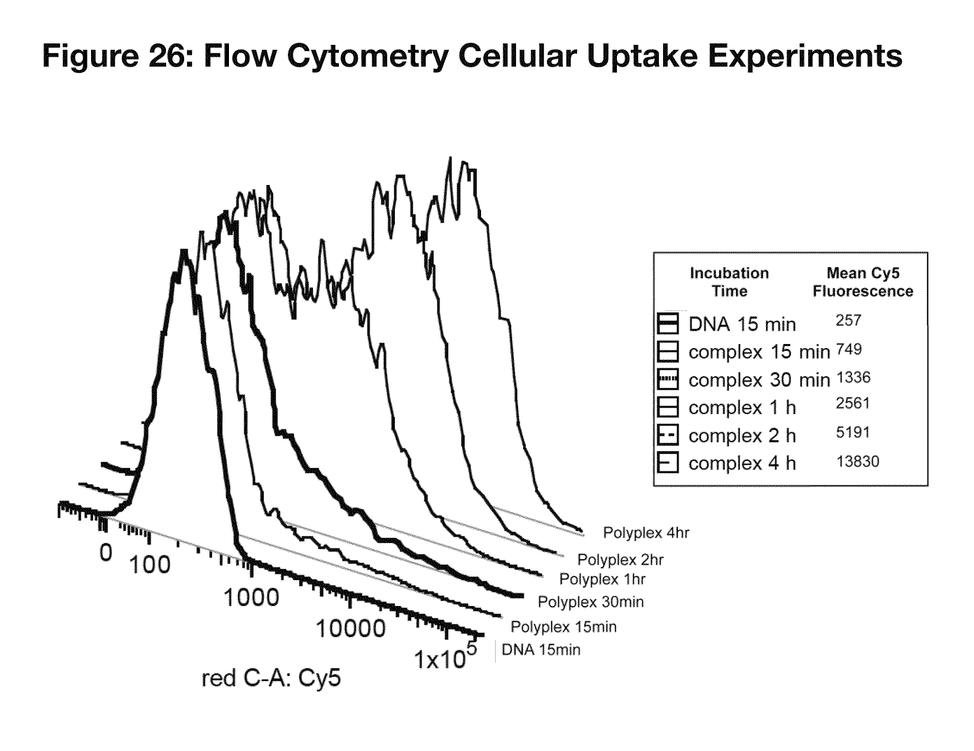

HCT-116 cells were seeded at 250000 cells per well in 12 well plates two days prior to transfection and grown in 1000 μL of media. On the day of transfection, 25 μL of N:P 10 polyplex, prepared as described in Example 47 using EGFP plasmid (pZs-Green; Clontech, Mountain View, Calif.) labeled with Cy5 (Mirus, Madison, Wis.), was added directly to media and incubated for up to 4 hr at 37° C. Cells incubated for 15 min at 37° C. with Cy5-plasmid DNA alone was used as a control. At each time point, the media was removed, cells washed once in PBS and CellScrub buffer (Genlantis, San Deigo, Calif.), trypsinized and resuspended in PBS with 1 μg of DAPI. A BD LSR-II (BD, NJ USA) flow cytometer was used to detect cell uptake of Cy5-plasmid DNA. FlowJo 8.3.3 software was used to analyze data, FIG. 26.

### Example 51

### Buffering Capacity of Poly(D/L Asp-DET) Polymer

Polymers dissolved in 150 mM NaCl were titrated with 0.01M HCl and first derivative analysis was performed on the pH-titration curves, FIG. 22. Poly(D/L Asp-DET) polymer exhibited two-step protonation (pH=7.8 and pH=5.4) draw these values onto the plot, get the exact value) while Poly(Lysine) only showed one protonation step (pH=8.9).

### Example 52

### Characterization of Poly(D/L Asp-DET) Polymer/DNA Complexation

Gel retardation of Poly(D/L Asp-DET)/DNA complexes demonstrating the effect of N:P ratio on DNA polyplex formation, FIG. 23A. Polyplex solutions were, prepared at different N:P ratios and 20 μg of pGL4 plasmid DNA. Samples were resolved on a 1% agarose gel and visualized by ethidium bromide. Ethidium bromide exclusion assay. Relative binding affinity of Poly(D/L Asp-DET) for plasmid DNA as measured by ethidium bromide fluorescence quenching. Data are the average ±SD (n=3), FIG. 23B.

### Example 53

### Size and Zeta-Potential of Polyplexes as a Function of N:P Ratio of Poly(Asp-DET)/DNA Polyplexes

DLS analysis of polyplex size for Poly(D/L Asp-DET) polymer between N:P ratios of 2.5 and 50 ranged from ˜170 to 53 nm (). Zeta-potential of polyplexes ranged from −40 mv to +45 mv (▪) (FIG. 24).

### Example 54

### Nuclease Protection of Plasmid DNA Complexed with Poly(D/L Asp-DET) Polymers

Half a ug of naked pGL4 plasmid DNA, or DNA complexed with Poly(D/L Asp-DET) at N:P ratio 5 and 10 was incubated with FBS at 37° C. At the indicated time points, samples were removed and incubated with or without heparin for 10 min at room temperature, and then resolved on 1% agarose/ethidium bromide gels, FIG. 25. Incubating naked DNA in 50% serum caused degradation within 30 min. In contrast, DNA complexed with Poly(D/L Asp-DET) polymer form polyplexes that protect DNA from degradation for at least one hr after incubation in serum.

### Example 55

### Internalization of Cy5-Labeled DNA Transfected with Poly(D/L Asp-DET) Polymers

Flow cytometry (Example 50) histogram of cell associated Cy5 fluorescence, FIG. 26. The leftmost peaks correspond to HCT-116 cells incubated with either media or DNA only. The right most peaks represent cell associated fluorescence after transfection with Poly(D/L Asp-DET)/Cy5-DNA polyplexes. Mean Cy5 fluorescence is shown in the table to the right.

### Example 56

### Comparison of Polyplex and PEG-Polyplex DNA Complexation Ability

Agarose gel retardation of Poly(D/L Asp-DET)/DNA Polyplexes at N:P 10 ratio. Samples were resolved on a 1% agarose gel and visualized by ethidium bromide, FIG. 27A. Both Polyplex and PEG-Polyplexes fully complexed 20 μg of plasmid DNA, and the addition of heparin demonstrated that all samples contained intact DNA. The relative binding affinity of Poly(D/L Asp-DET) Polyplexes or PEG-Polyplexes for plasmid DNA was measured by ethidium bromide fluorescence quenching, FIG. 27B. Data are the average ±SD (n=3). Addition of PEG to Polyplexes had minimal effect on Poly(D/L Asp-DET) polymer binding affinity to DNA.

### Example 57

### Physiochemical Characterization and Comparison of Polyplexes and PEG-Polyplexes

DLS and Zeta-potential analysis of Polyplexes and PEG-Polyplexes at N:P 10, FIG. 28A. DLS measurement determined PEG-polyplexes to be approximately 15 nm larger in diameter than Polyplexes. Addition of PEG to Polyplexes resulted in near neutral zeta-potential. TEM of Poly(D/L Asp-DET)/DNA N:P 10 PEG-Polyplexes, FIG. 28B. PEG-Polyplexes showed similar morphology to polyplexes and were also smaller than 100 nm. Bar=200 nm.

### Example 58

### Morphologies of PEG-Polyplexes Using Either Diblock Polymers or Cationi Homopolymer

PEG-Polyplexes using Cationic Poly(D/L Asp-DET) Homopolymer were formulated as described in Example 47. PEG-Polyplexes using Diblock Polymers were prepare by adding equal volumes of N3-PEG12k-b-P(Asp-DET)100 (D/L) solution (dissolved in dH₂O) and plasmid DNA solution (200 μg/mL in dH₂O) at the appropriate N:P ratio. Polymer was added to DNA solution, for a final volume of 200 μL, and incubated at room temperature for at least 30 min to allow polyplex formation. Samples were prepared for TEM as described in Example 6. TEM of Poly(D/L Asp-DET)/DNA N:P 10 PEG-Polyplexes, FIG. 28A showed uniform sized nanoparticles smaller than 100 nm. TEM of N3-PEG12k-b-P(Asp-DET)100 (D/L) N:P 5 PEG-Polyplexes is shown in FIG. 28B.

### Example 59

### Size Analysis and Morphologies of Polyplexes at Various N:P Ratios

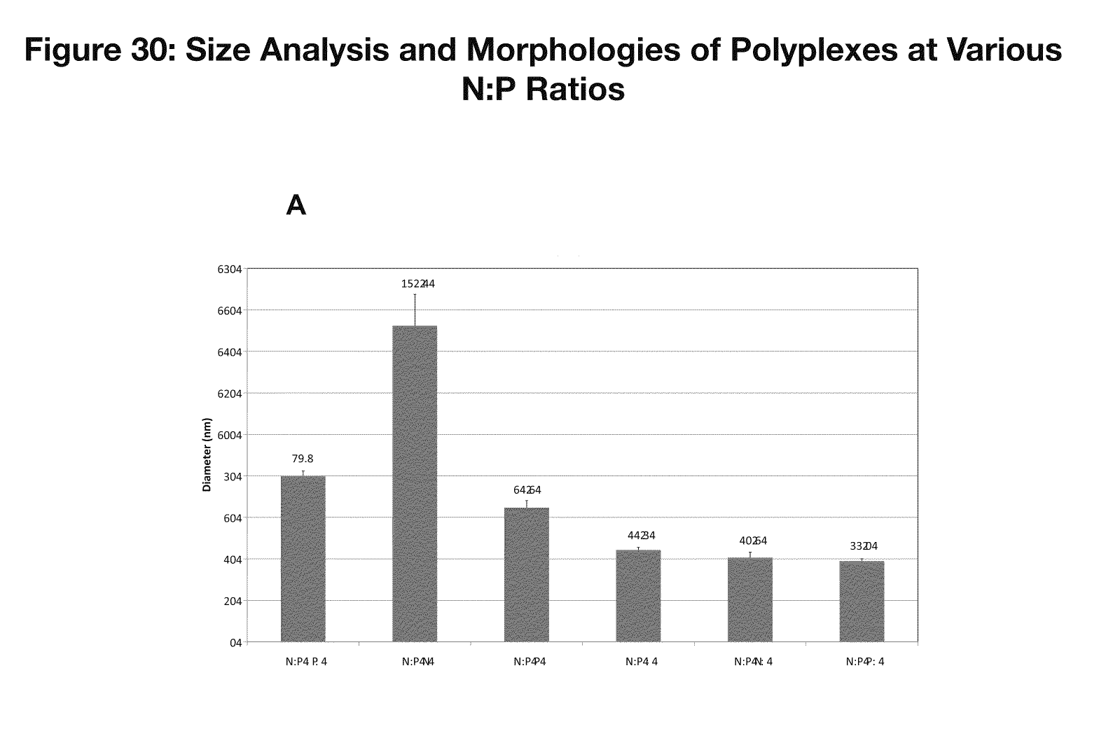

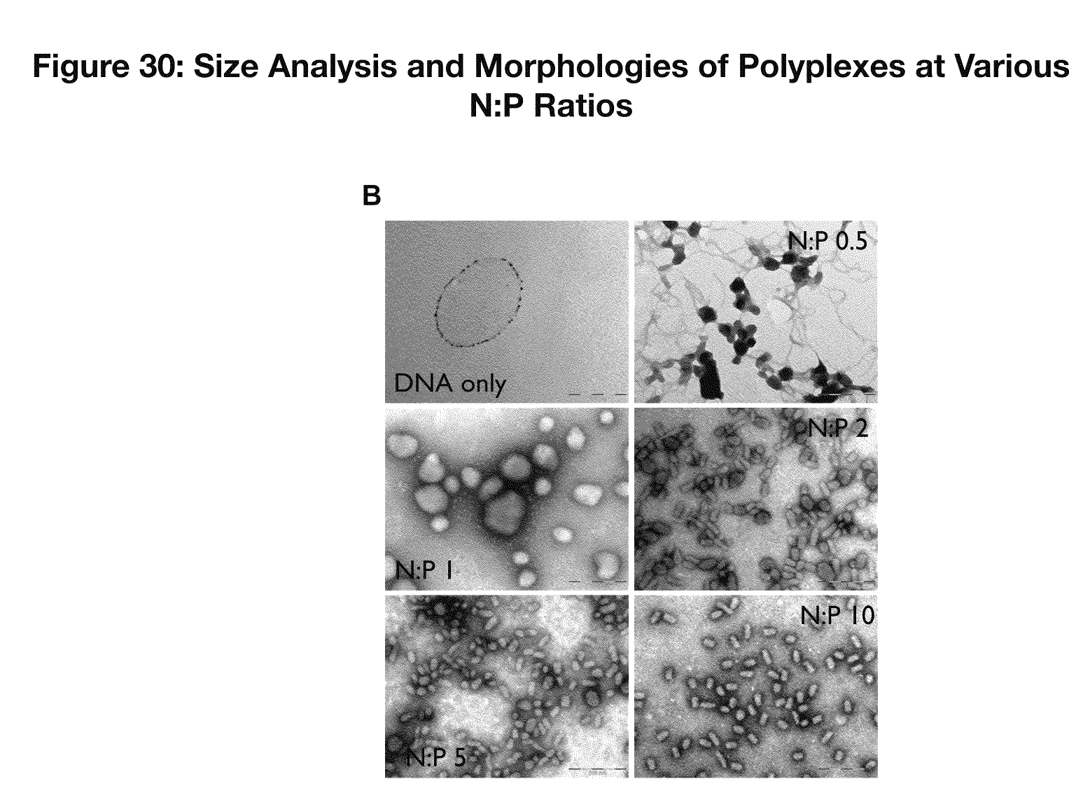

Dynamic light scattering analysis (Example 38 & 48A) of polyplex size for the Poly(D/L Asp-DET) polymer between N:P ratios of 0.5 and 20 ranged from ˜150 to ˜40 nm, FIG. 30A. TEM analysis (Example 40) of Poly(D/L Asp-DET) polymer interaction with plasmid DNA. Uniform sized structures less than 100 nm in size formed at N:P ratios greater than 1, FIG. 30B.

### Example 60

### In Vivo Studies Using D/L Asp-DET and Various PEG Polymers

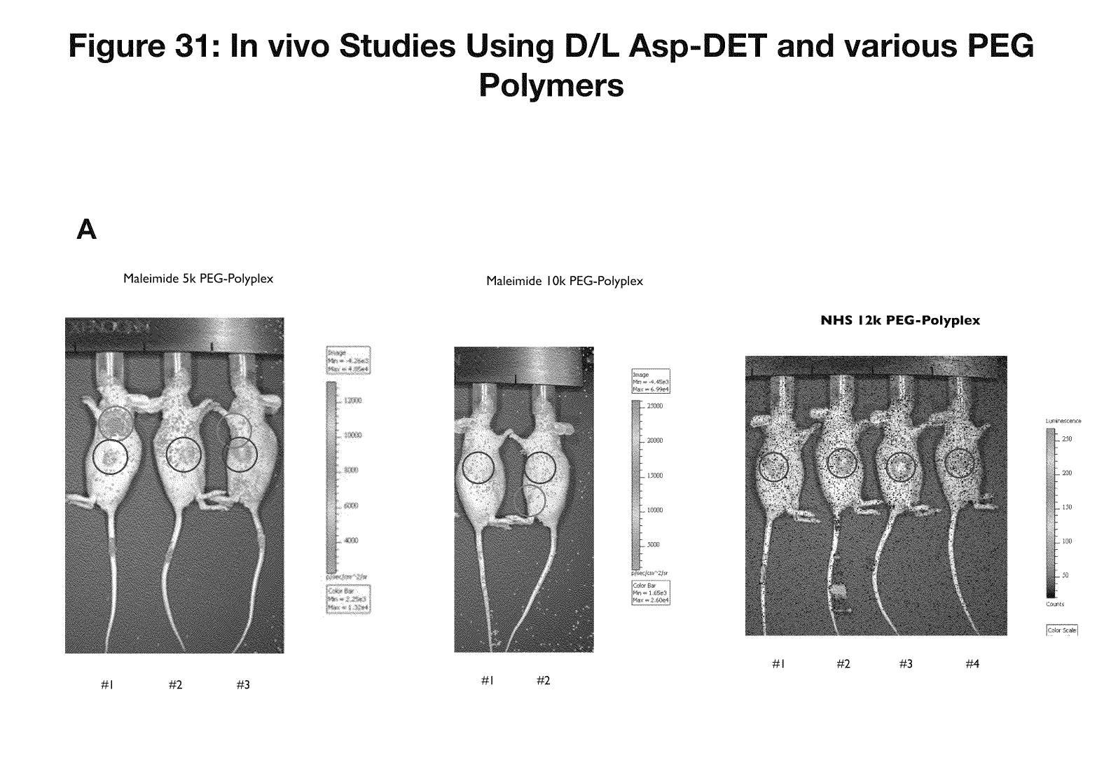

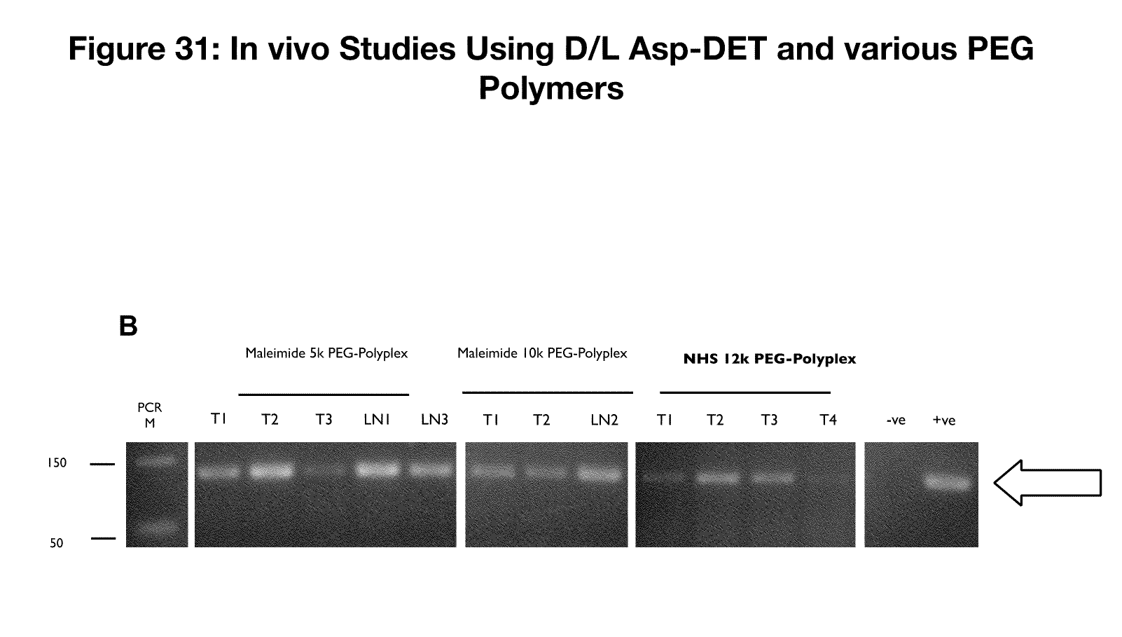

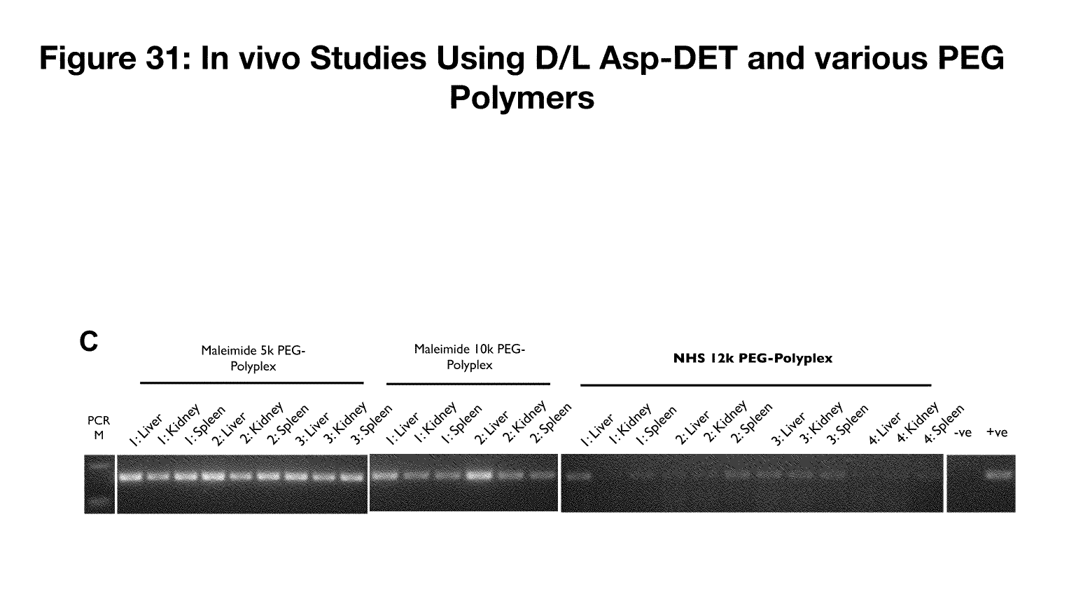

Various Post-PEG Poly(D/L Asp-DET)/DNA polyplexes, containing 20 ug of pGL4-luciferase plasmid, were administered to HCT-116 tumor bearing nude mice by tail vein administration. IVIS images of mice 72 hours after IV injection, FIG. 31A. HCT-116 tumors are circled dark grey and lymph nodes are circled light grey. PCR results of tumor and lymph node tissues, FIG. 31B and other organs, FIG. 31C. Plasmid DNA accumulation was demonstrated in both tumors and lymph nodes using all PEG-Polyplexes. Plasmid DNA accumulation in liver, kidney and spleen was the lowest using NHS 12 k PEG-Polyplexes.

### Example 61

### Luciferase Expression of Colon Cancer Cells Transiently Transfected with Poly(D/L Asp-DET)/DNA Polyplexes and PEG-Polyplexes

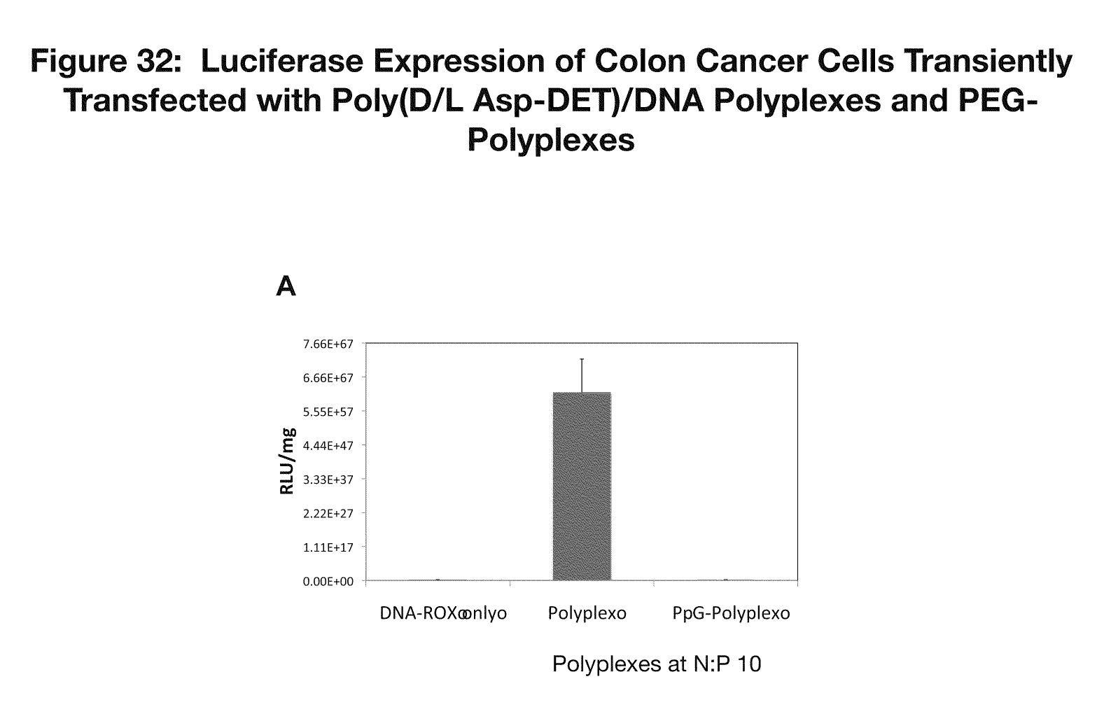

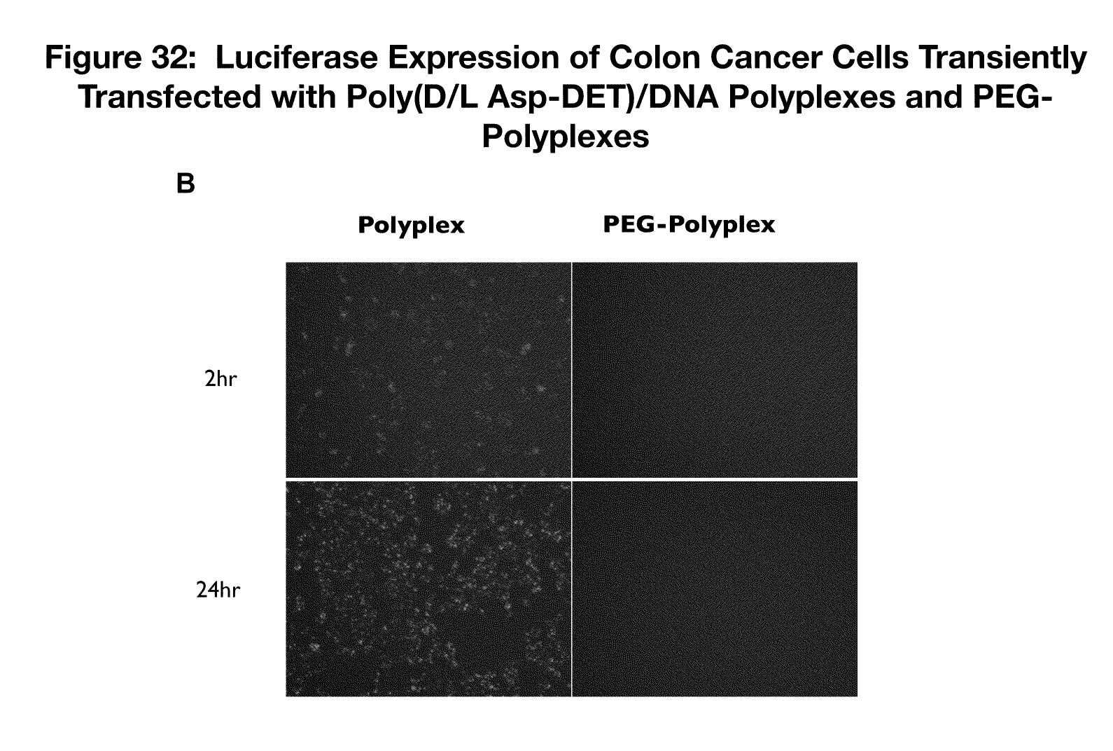

HCT-116 cells were transfected in triplicate in 96-well plates with Poly(D/L Asp-DET)/DNA polyplexes or NHS 12 k PEG-Polyplexes, containing rhodamine-labeled firefly luciferase pGL4 plasmid DNA at a final DNA concentration of 0.25 μg DNA per well. Twenty-four hr after transfection, luciferase activity for each sample was determined and normalized to protein content. All results are representative of triplicate experiments. Luciferase activity was only observed in cells treated with Polyplexes, FIG. 32A. Fluorescent microscopy showed that rhodamine-labeled DNA (red) was present in both the nucleus and cytoplasm of cells treated with Polyplexes, FIG. 32B. No DNA could be detected on or in cells treated with PEG-Polyplexes suggesting that PEG-Polyplexes did not enter cells, FIG. 32B. ×10 magnification.

### Example 62

### Strategy to Attach Targeting Groups to PEG-Polyplexes

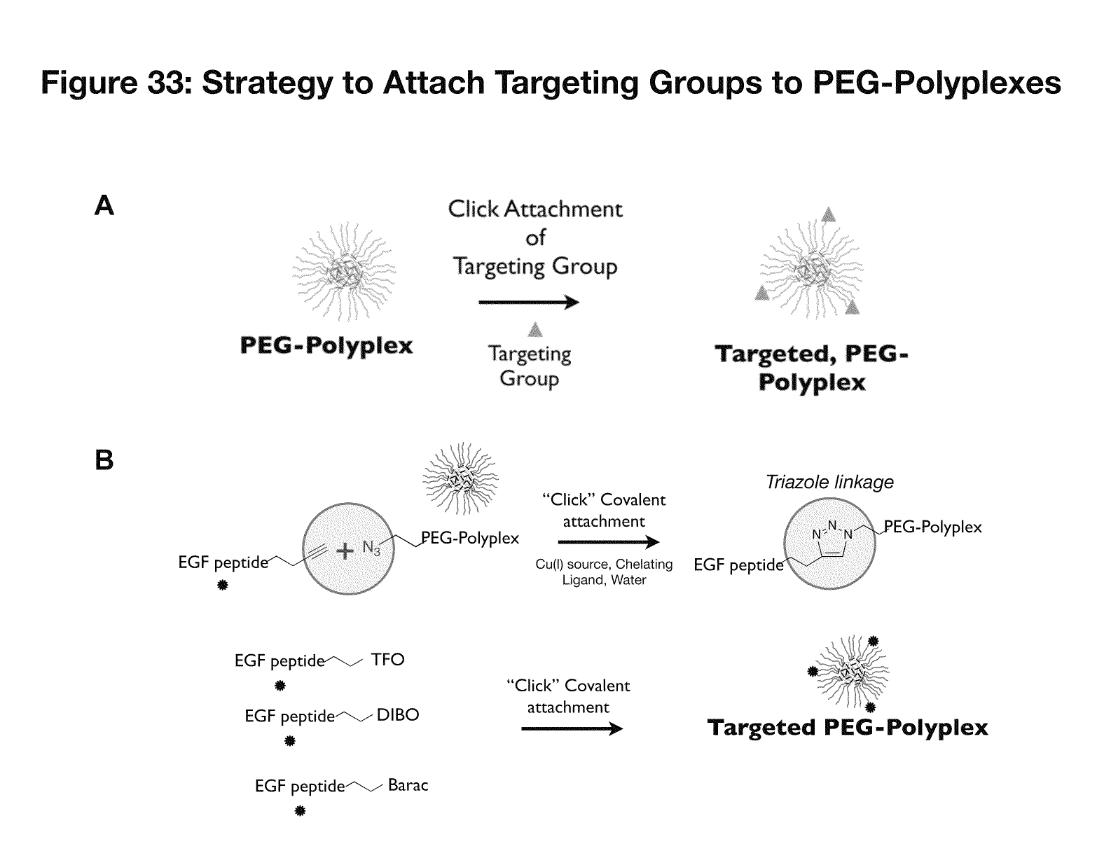

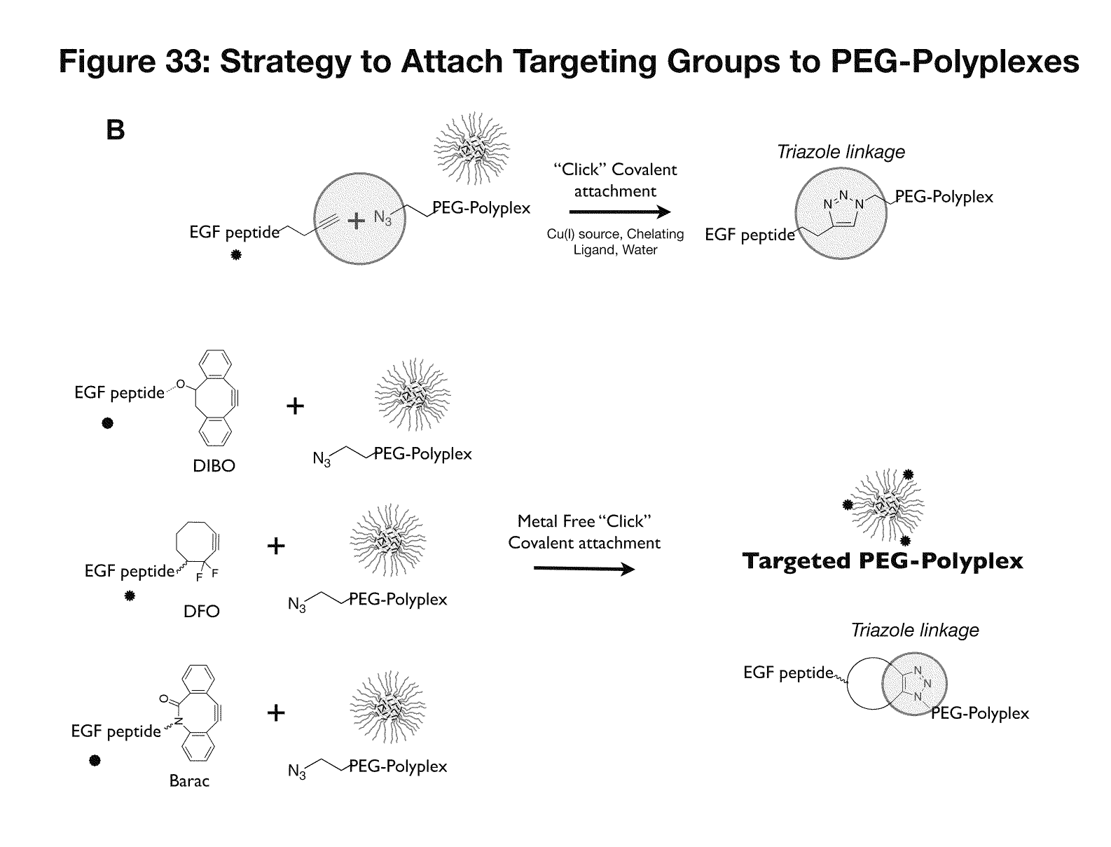

Poly(d/l Asp-DET)/DNA NHS 12 k PEG-Polyplexes were prepare as described in Example 36. Targeting groups containing functional moieties for Click chemistry attachment to Azides on NHS-12 k PEG were incubated, 1 hr to overnight, with PEG-Polyplexes to make targeted PEG-Polyplexes, (See FIG. 33A for schematic). FIG. 33B shows EGF peptide linked with various reactive alkynes or oxanorbornadiene carboxylic acid derivatives.

### Example 63

### Attachment of EGF Peptide Targeting Groups to PEG-Polyplexes

Poly(d/l Asp-DET)/DNA NHS 12 k PEG-Polyplexes were prepare as described in Example 36. PEG-Polyplexes were ultrafiltrated using a Vivaspin 500 100,000 MWCO filters (Sartorius Stedim Biotech GmbH, Germany), and concentrated PEG-Polyplexes were diluted with dH₂O to a final volume of 427.5 μL to achieve equal volumes for all samples. Forty μL of Alkyne-EGF peptide (LARLLT, 5 mg/mL in dH₂O), 2.5 μL of CuSO4 (5 mg/mL in dH₂O), 5 μL, of THPTA ligand (2.17 mg/100 μL in dH₂O) and 25 μL of sodium ascorbate (20 mg/mL in dH₂O), was added to PEG-Polyplex solutions and incubated overnight at room temperature with shaking.

### Example 64

### Localization and Expression of Luciferase Plasmid DNA in Colon Cancer Cells Transfected with Copper Click Chemistry Created EGF Targeted PEG-Polyplexes

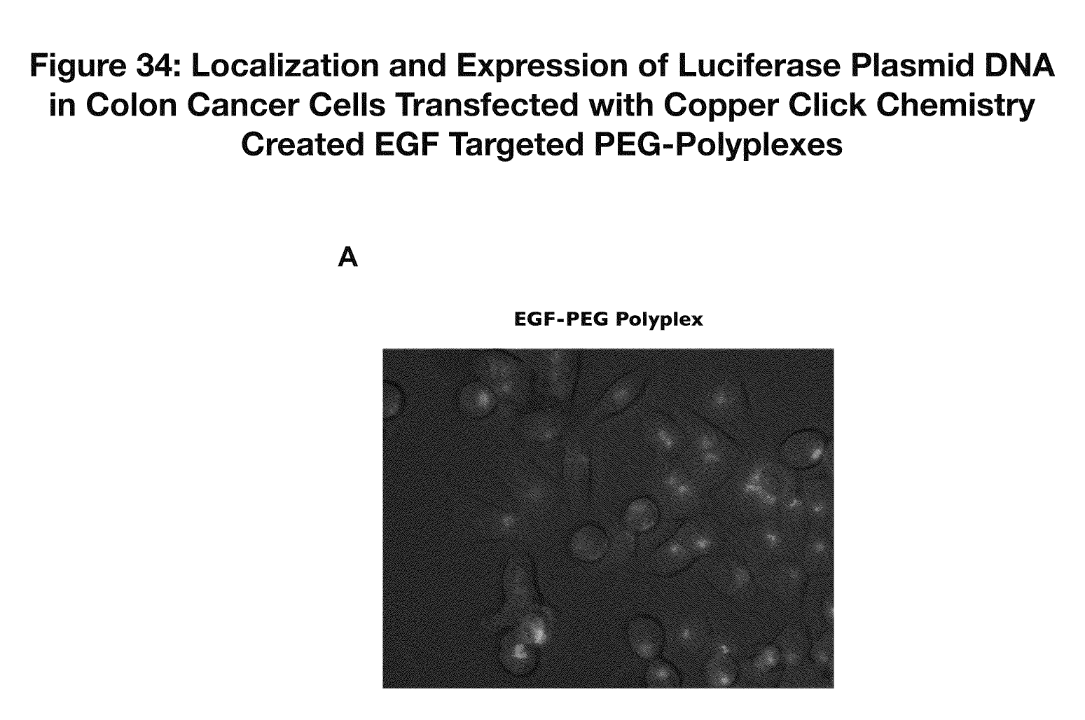

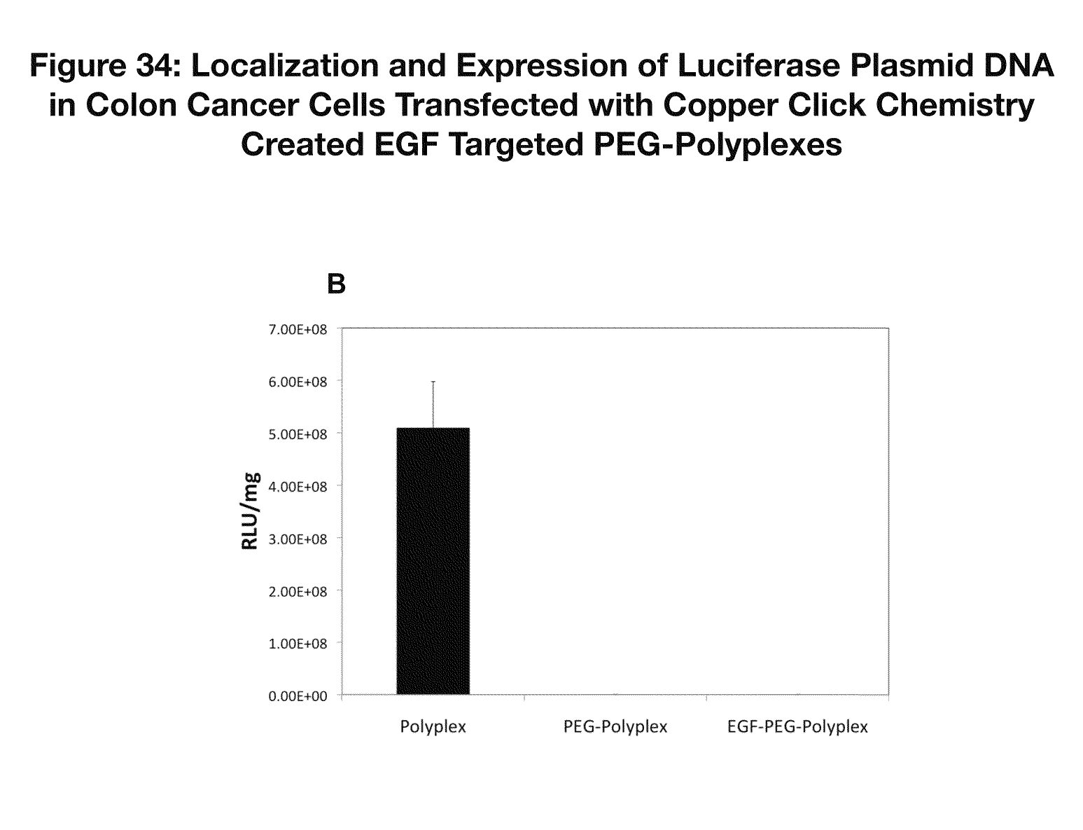

HCT-116 cells were transfected in triplicate in 96-well plates with Poly(D/L Asp-DET)/DNA polyplexes, NHS 12 k PEG-Polyplexes (Example 47) or targeted EGF-PEG-Polyplexes (Example 63), containing rhodamine-labeled firefly luciferase pGL4 plasmid DNA at a final DNA concentration of 0.25 μg DNA per well. Twenty-four hr after transfection, fluorescent microscopy showed that rhodamine-labeled DNA (red) was present in both the nucleus and cytoplasm of cells treated with EGF-PEG-Polyplexes, FIG. 34A. ×40 magnification. However, luciferase activity was only observed in cells treated with Polyplexes, FIG. 34B.

### Example 65

### Localization and Expression of Luciferase Plasmid DNA in Colon Cancer Cells Transfected with Copper Click Chemistry Created EGF Targeted PEG-Polyplexes

HCT-116 cells were transfected in triplicate in 96-well plates with Poly(D/L Asp-DET)/DNA polyplexes, NHS 12 k PEG-Polyplexes or targeted EGF-PEG-Polyplexes, containing rhodamine-labeled firefly luciferase pGL4 plasmid DNA at a final DNA concentration of 0.25 μg DNA per well. Twenty-four hr after transfection, fluorescent microscopy showed that rhodamine-labeled DNA (red) was present in both the nucleus and cytoplasm of cells treated with EGF-PEG-Polyplexes, FIG. 34A. ×40 magnification. However, luciferase activity was only observed in cells treated with Polyplexes, FIG. 34B.

### Example 66

### Adverse effects of Copper Click Reagents on DNA

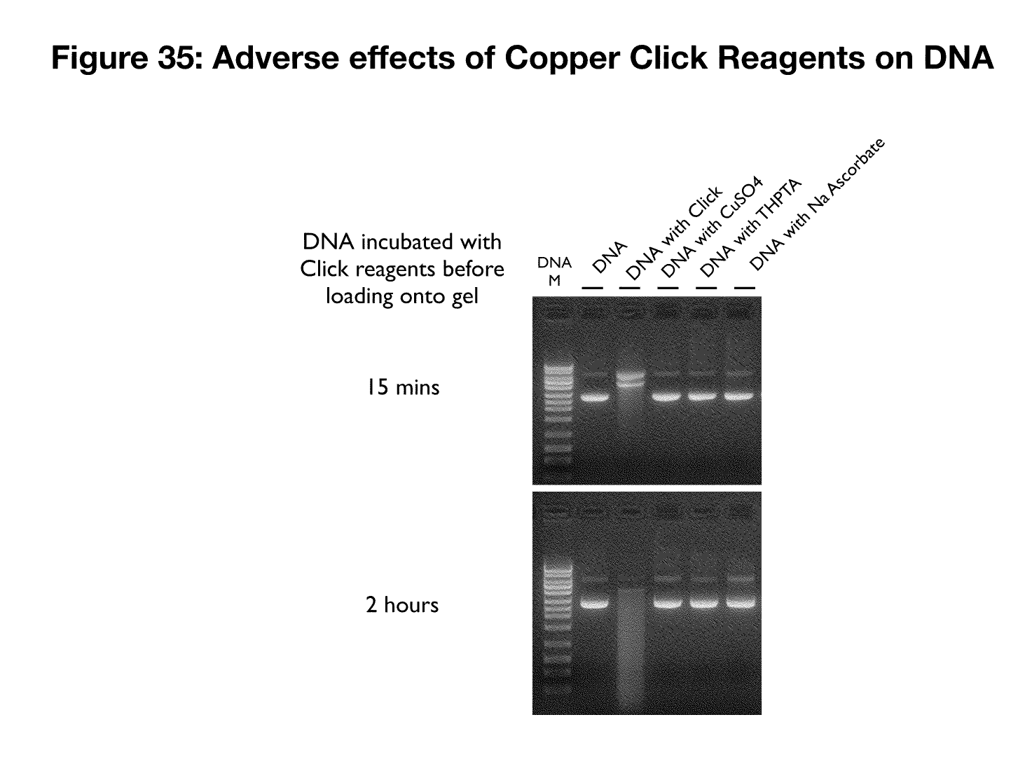

Twenty μg of firefly luciferase pGL4 plasmid DNA was diluted with dH₂O to a final volume of 427.5 μL to achieve equal volumes for all samples. Two and a half μL of CuSO4 (5 mg/mL in dH₂O), 5 μL of THPTA ligand (2.17 mg/100 μL in dH₂O; Hong, V.; Presolski, S. I.; Ma, C.; Finn, M. G. Angew. Chem., Int. Ed. 2009, 48, 9879-9883) and 25 μL of sodium ascorbate (20 mg/mL in dH₂O), was added to PEG-Polyplex solutions and incubated for 2 hours at room temperature, FIG. 35. DNA samples were also incubated with reagents separately for 2 hours. Samples (0.5 μg of each) were resolved on a 1% agarose gel and visualized by ethidium bromide. While THPTA ligand and CuSO₄ has no effect on DNA, sodium ascorbate caused gradual DNA degradation. Combining all Click reagents caused rapid DNA degradation, FIG. 35.

### Example 67

### Attachment of EGF Peptide Targeting Groups to PEG-Polyplexes

Poly(d/l Asp-DET)/Rhodamine-DNA NHS 12 k PEG-Polyplexes were prepare as described in Example 36. PEG-Polyplexes were ultrafiltrated using a Vivaspin 500 100,000 MWCO filters (Sartorius Stedim Biotech GmbH, Germany), and concentrated PEG-Polyplexes were diluted with dH₂O to a final volume of 100 μL to achieve equal volumes for all samples. Four hundred μL of 3-Methoxycarbonyl-7-oxa-norbornadiene-2-carbonyl-EGF peptide (LARLLT, 5 mg/mL in dH₂O), was added to PEG-Polyplex solutions and incubated overnight at room temperature with shaking.

### Example 68

### Gel Retardation and Size Analysis of EGF Targeted PEG-Polyplexes Created by Copper free Click Chemistry

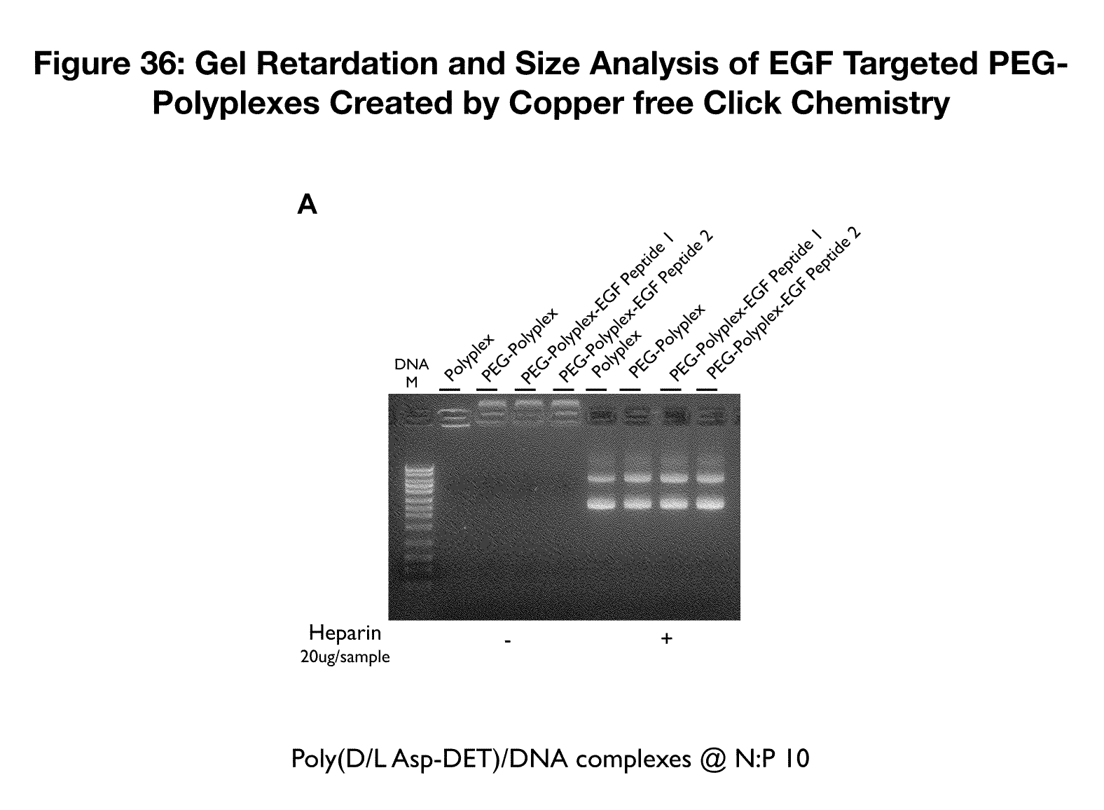

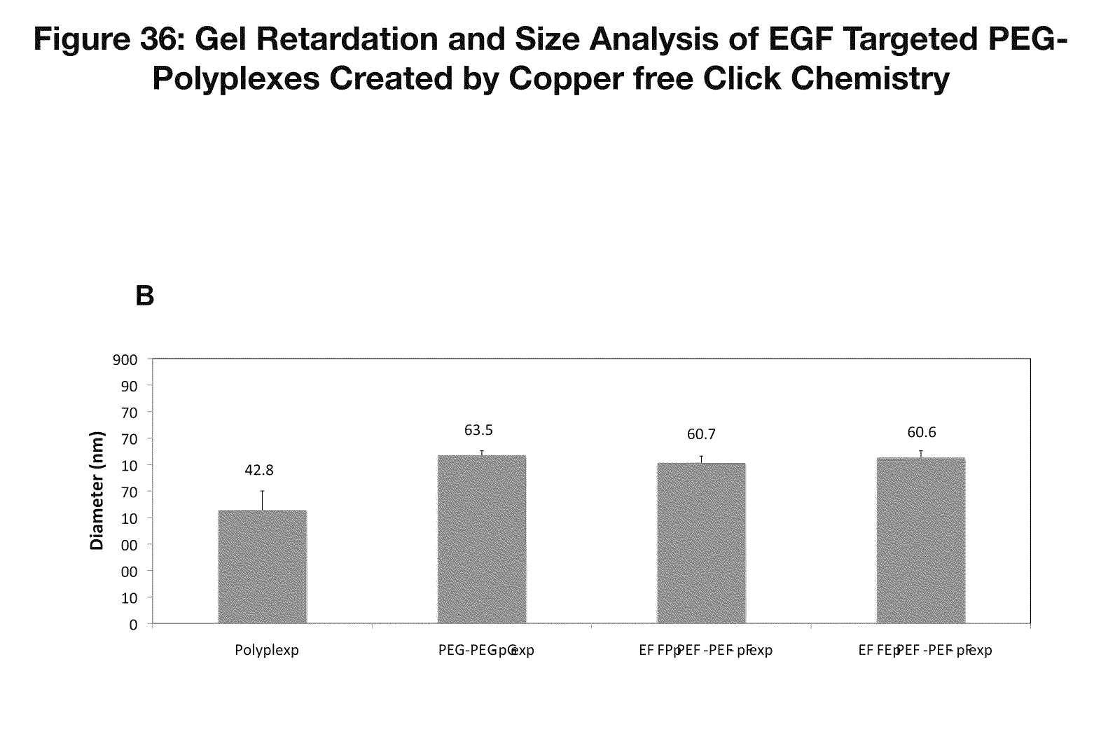

Agarose gel retardation of Poly(D/L Asp-DET)/DNA polyplexes, NHS 12 k PEG-Polyplexes or Copper free Click targeted EGF-PEG-Polyplexes, at N:P 10 ratio. Samples were resolved on a 1% agarose gel and visualized by ethidium bromide, FIG. 36A. All Polyplex samples fully complexed 20 μg of plasmid DNA, and the addition of heparin demonstrated that all samples contained intact DNA. Dynamic light scattering analysis of non- and PEG polyplexes at N:P 10, prepared according to Example 67, FIG. 36B.

### Example 69

### Morphologies and Salt stability of EGF Targeted PEG-Polyplexes Created by Copper Free Click Chemistry

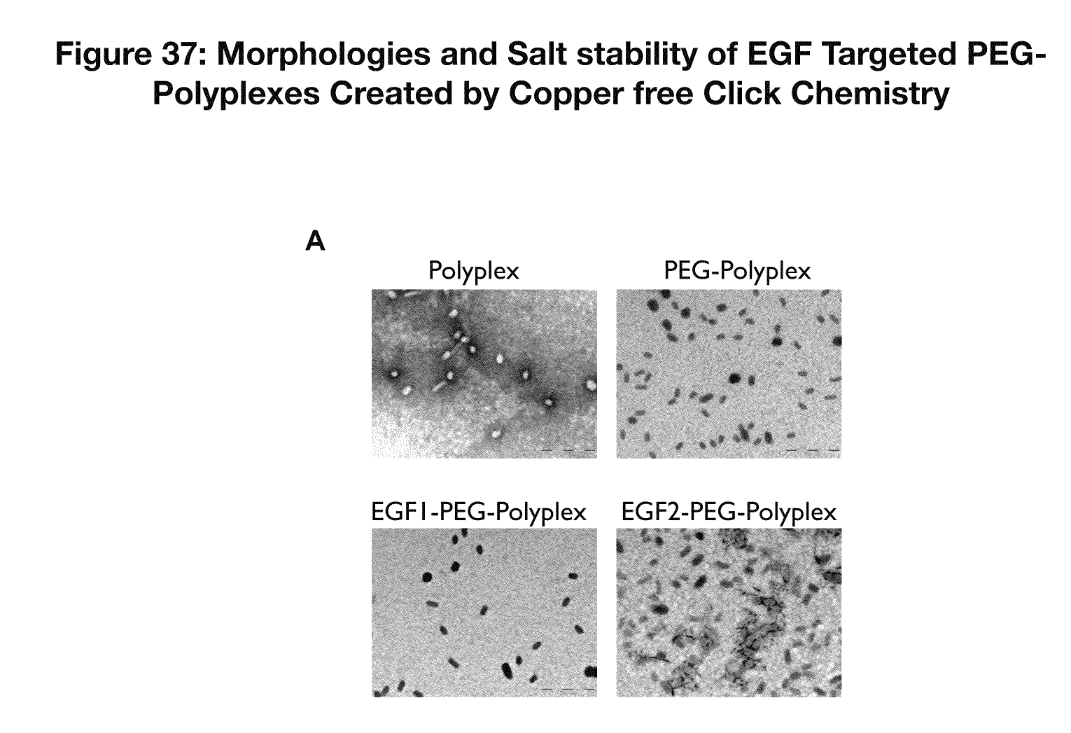

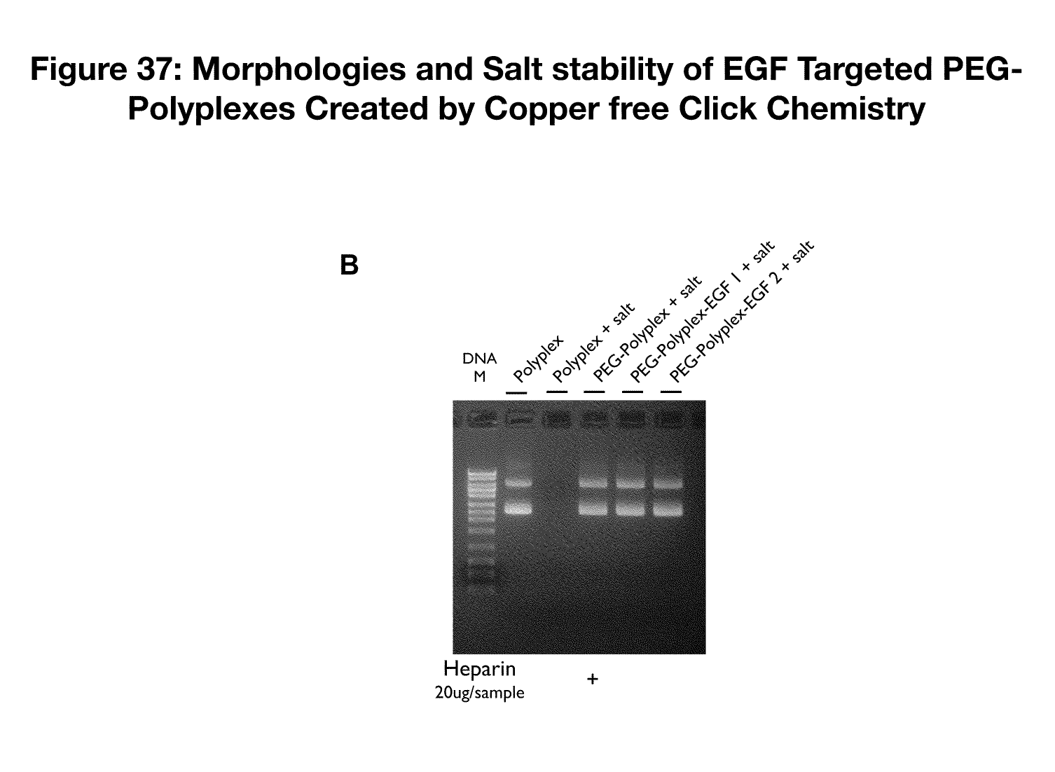

TEM analysis of EGF PEG-Polyplexes created by copper free Click chemistry demonstrated uniform sized structures less than 100 nm in size that were similar in morphology to Polyplexes and PEG-Polyplexes, FIG. 37A. Polyplex samples were centrifuged following salt addition and incubation as described in Example 44. After centrifugation, heparin was added to supernatant to dissociate DNA from polymers and the samples were resolved on a 1% agarose/ethidium bromide gel, shown in FIG. 37B. Only PEG-Polyplex and EGF PEG-Polyplex samples remained in solution, and contained intact DNA following the addition of salt.

### Example 70

### Localization of Fluorescently Labeled DNA in Colon Cancer Cells Transfected with Targeted EGF PEG-Polyplexes

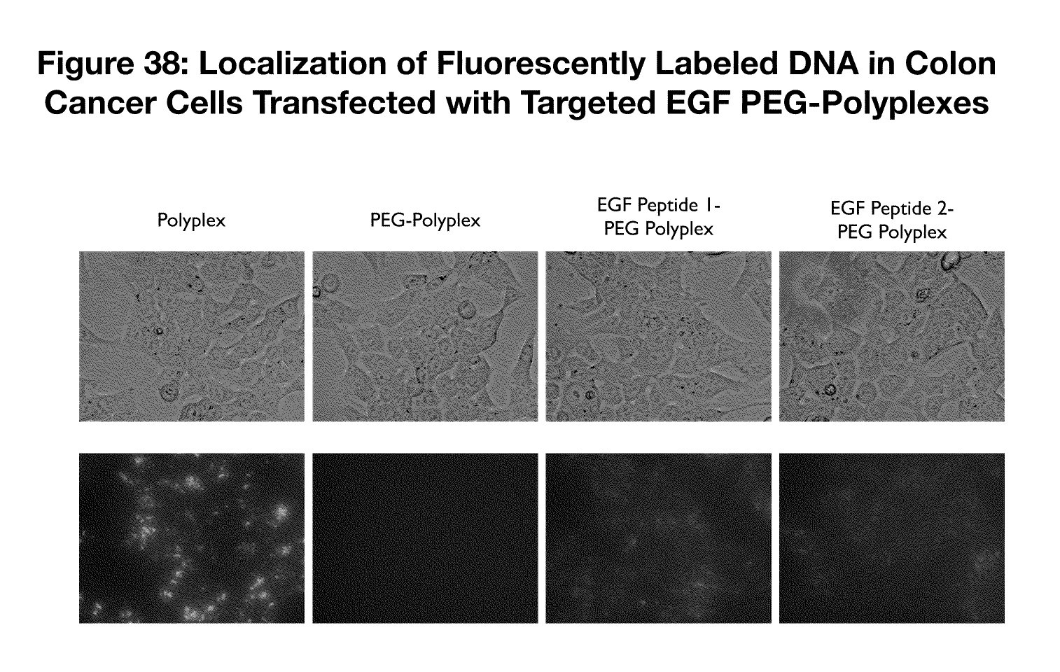

HCT-116 cells were transfected in triplicate in 96-well plates with Poly(D/L Asp-DET)/DNA polyplexes, NHS 12 k PEG-Polyplexes or targeted EGF-PEG-Polyplexes, containing rhodamine-labeled firefly luciferase pGL4 plasmid DNA at a final DNA concentration of 0.25 μg DNA per well. Twenty-four hr after transfection, fluorescent microscopy showed that rhodamine-labeled DNA (red) was present in both the nucleus and cytoplasm of cells treated with EGF-PEG-Polyplexes, FIG. 38. ×40 magnification.

### Example 71

### Expression of Luciferase Plasmid DNA in Colon Cancer Cells Transfected with Copper Free Click Chemistry Created EGF Targeted PEG-Polyplexes

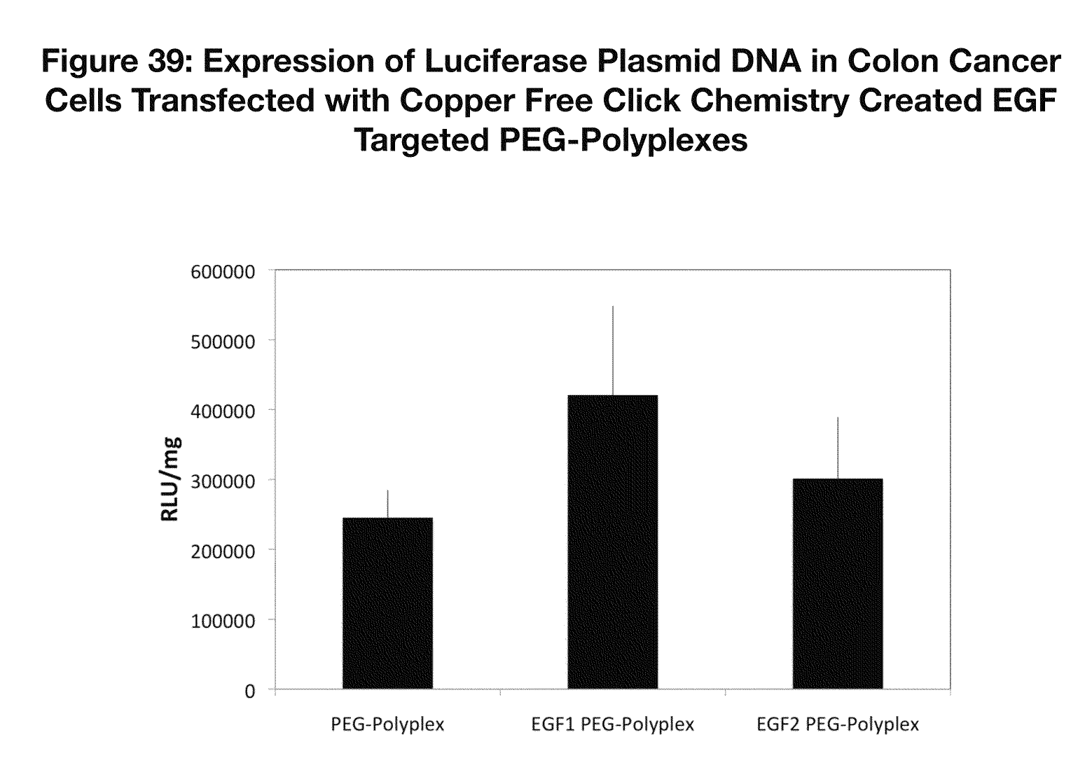

HCT-116 cells were transfected in triplicate in 96-well plates with Poly(D/L Asp-DET)/DNA NHS 12 k PEG-Polyplexes or targeted EGF-PEG-Polyplexes, containing rhodamine-labeled firefly luciferase pGL4 plasmid DNA at a final DNA concentration of 0.25 μg DNA per well. Twenty-four hr after transfection, luciferase activity for each sample was determined and normalized to protein content. All results are representative of triplicate experiments. Luciferase activity was only observed in cells treated with Targeted EGF PEG-Polyplexes, FIG. 39.

### Example 72

### In Vivo Studies Using EGF Targeted PEG-Polyplexes

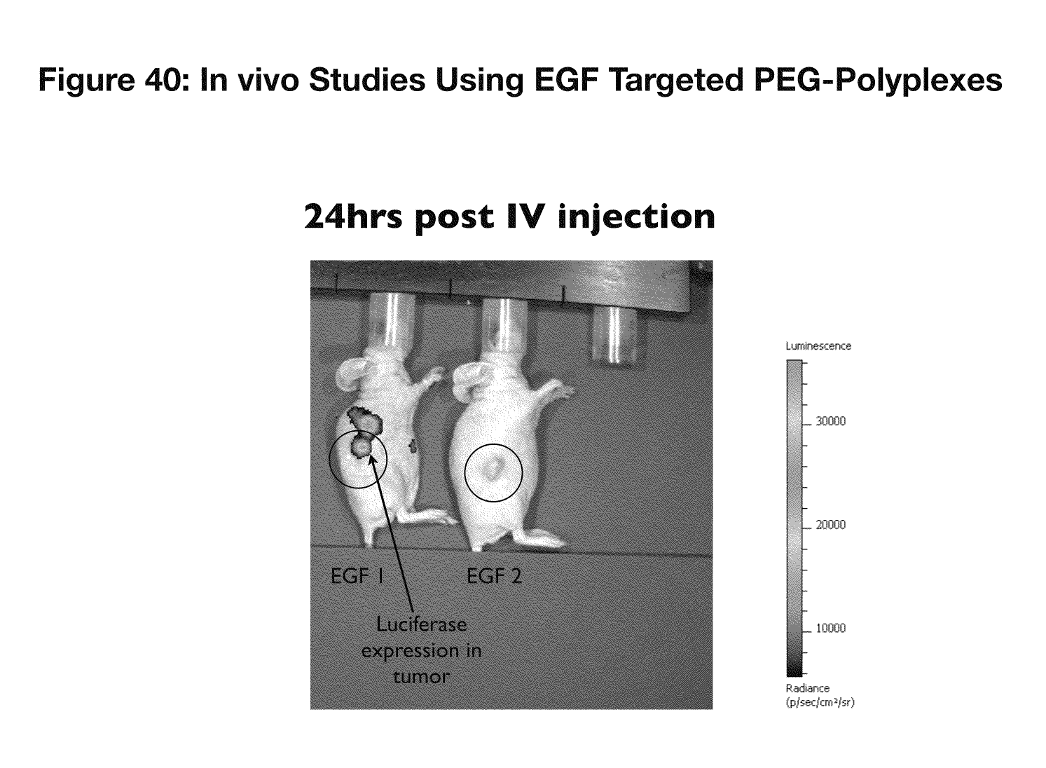

Various EGF Targeted PEG-Polyplexes, containing 20 μg of pGL4-luciferase plasmid, were administered to HCT-116 tumor bearing nude mice by tail vein administration. IVIS images of mice 24 hours after IV injection, FIG. 40. HCT-116 tumors are circled black. Luciferase plasmid DNA expression was demonstrated in tumors and lymph nodes of a mouse injected with EGF1-PEG-Polyplex.

### Example 73

### Comparison of DNA Complexation Using Either PEI or Poly(D/L Asp-DET) Polymers

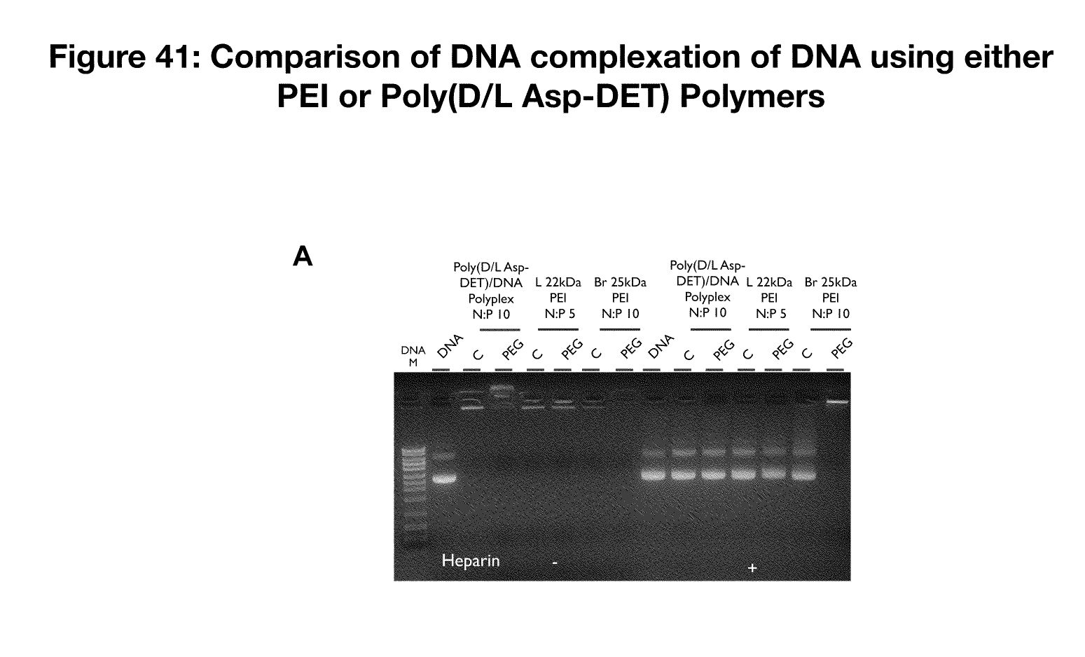

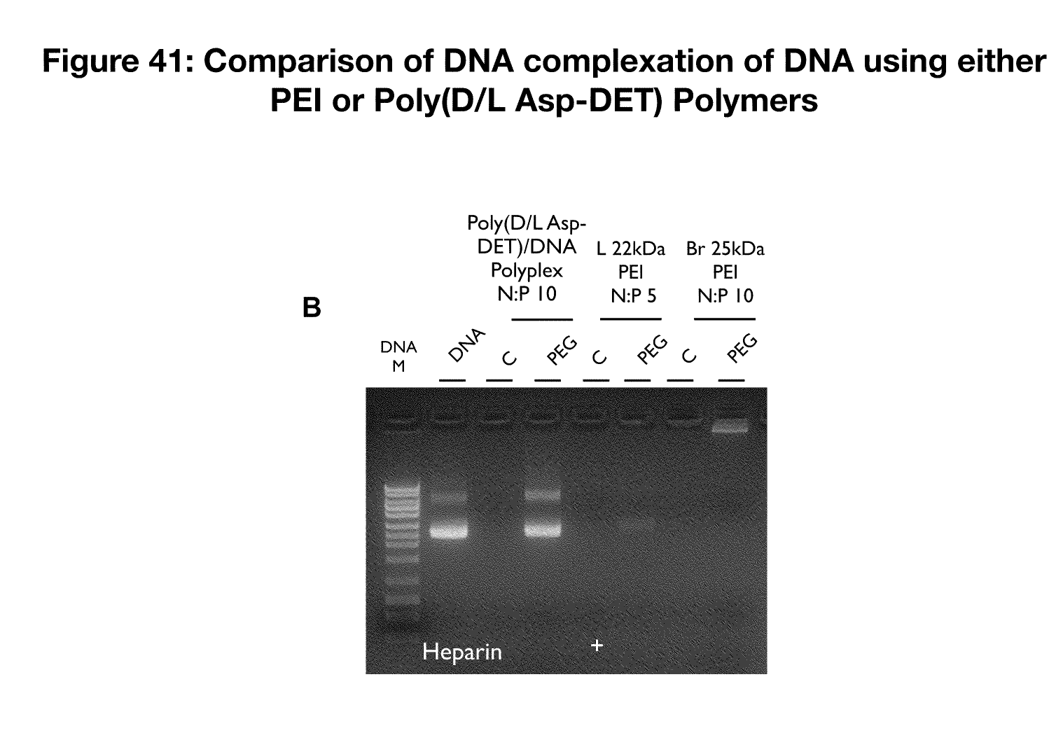

PEG-Polyplexes using Poly(D/L Asp-DET) polymer were formulated as described in Example 47. PEG-Polyplexes using 22 kDa linear or 25 kDa branched PEIs were prepared by adding equal volumes of PEI solution (dissolved in dH₂O) and plasmid DNA solution (200 μg/mL in dH₂O) at the appropriate N:P ratio. PEI polymer was added to DNA solution, for a final volume of 200 μL, and incubated at room temperature for at least 30 min to allow polyplex formation. PEG-PEI Polyplexes were prepared as described in Example 47. Samples (0.5 μg of DNA) were electrophoresed in a 1% agarose gel as described for the gel retardation experiment (Example 48 & 49), FIG. 41A. Unlike PEIs, the covalent attachment of PEG did not affect binding affinity of Poly(D/L Asp-DET) polymer to DNA FIG. 41A. Polyplex samples were centrifuged following salt addition and incubation as described in Example 44. After centrifugation, heparin was added to supernatant to dissociate DNA from polymers and the samples were resolved on a 1% agarose/ethidium bromide gel, shown in FIG. 41B. Only PEG-Poly(D/L Asp-DET)/DNA Polyplex samples remained in solution and contained intact DNA following the addition of salt. Poly(D/L Asp-DET) polymers allow for necessary PEG coverage to avoid salt induced aggregation.

### Example 74

### Co-Complexation of DNA using Linear PEI and Poly(D/L Asp-DET) Polymers

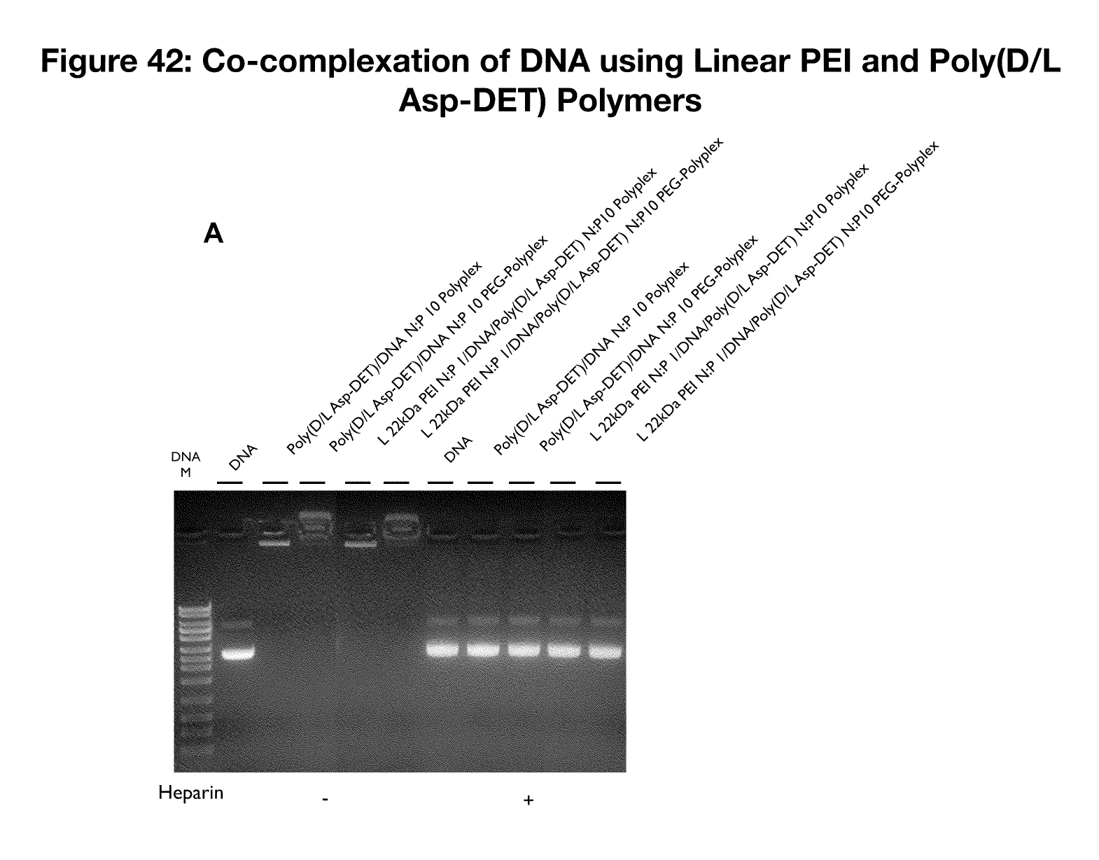

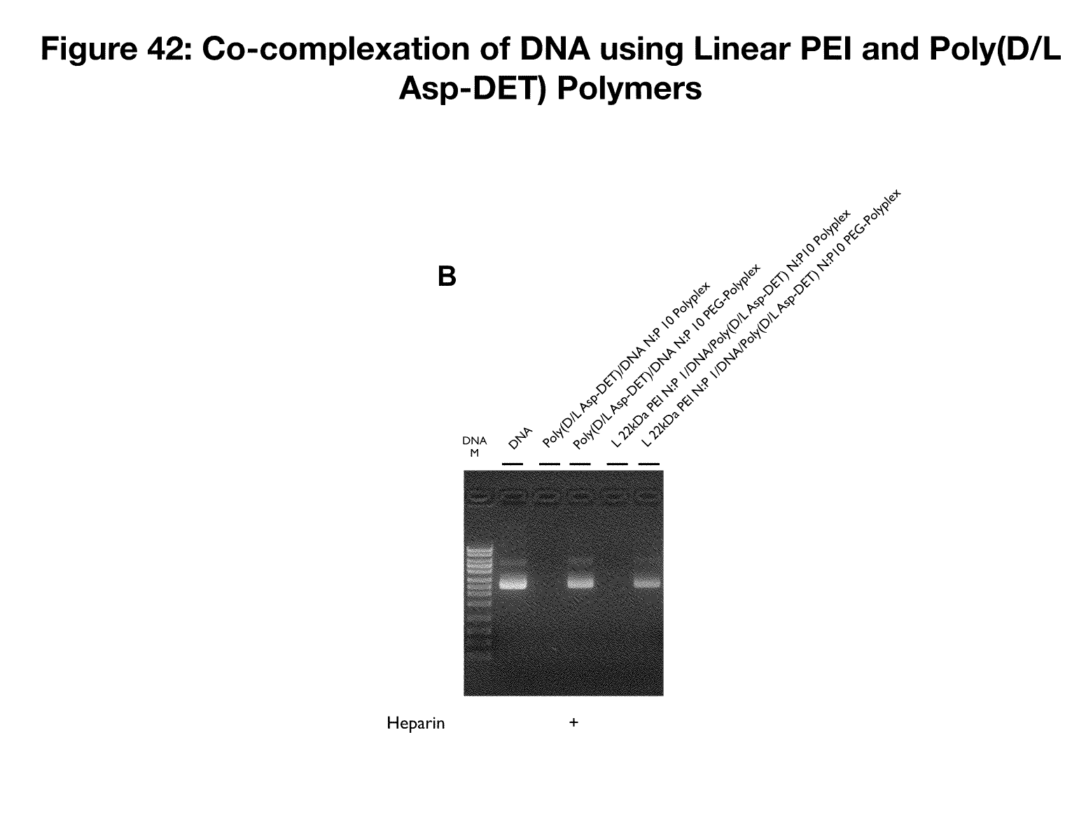

PEG-Polyplexes using Poly(D/L Asp-DET) polymer were formulated as described in Example 47. PEG-Polyplexes using both 22 kDa linear PEI and Poly(D/L Asp-DET) were prepared by adding PEI solution (dissolved in dH₂O) to plasmid DNA solution (200 μg/mL in dH₂O) at N:P ratio 1 and incubating at room temperature for at least 30 min. Poly(D/L Asp-DET) polymer solution (dissolved in dH₂O) was then added and incubated for an additional 30 min at room temperature to complete polyplex formation. PEG-PEI/Poly(D/L Asp-DET) Polyplexes were prepared as described in Example 47. Samples (0.5 μg of DNA) were electrophoresed in a 1% agarose gel as described for the gel retardation experiment (Example 48 & 49), FIG. 42A. The covalent attachment of PEG was not affected in co-complexed samples FIG. 42A. Polyplex samples were centrifuged following salt addition and incubation as described in Example 44. After centrifugation, heparin was added to supernatant to dissociate DNA from polymers and the samples were resolved on a 1% agarose/ethidium bromide gel, shown in FIG. 42B. PEG-PEI/Poly(D/L Asp-DET)/DNA Polyplex samples remained in solution and contained intact DNA following the addition of salt.

### Example 75

### Formulation of Poly(D/L Glu-DET) Polymer/Nucleic Acid Polyplexes

Poly(D/L Glu-DET)/DNA polyplexes were prepared by adding equal volumes of Poly(D/L Glu-DET) solution (dissolved in dH₂O) and plasmid DNA solution (200 μg/mL in dH₂O) at the appropriate N:P ratio. Polymer was added to DNA solution, for a final volume of 200 μL, and incubated at room temperature for at least 30 min to allow polyplex formation. PEG-polyplexes were formed by incubating 200 μL of Poly(D/L Glu-DET)/DNA N:P 10 polyplexes with 50 μL of Azide-12 k PEG-NHS (60 mg/mL in dH₂O, refs) for 3 hr with shaking at room temperature. Un-reacted PEG was removed by ultrafiltration using a Vivaspin 500 100,000 MWCO filters (Sartorius Stedim Biotech GmbH, Germany), and PEG-polyplexes were diluted with dH₂O to a final volume of 200 μL to achieve equal volumes for all samples.

### Example 76

### Complexation Studies Studies Using Poly(D/L Glu-DET) Polymers

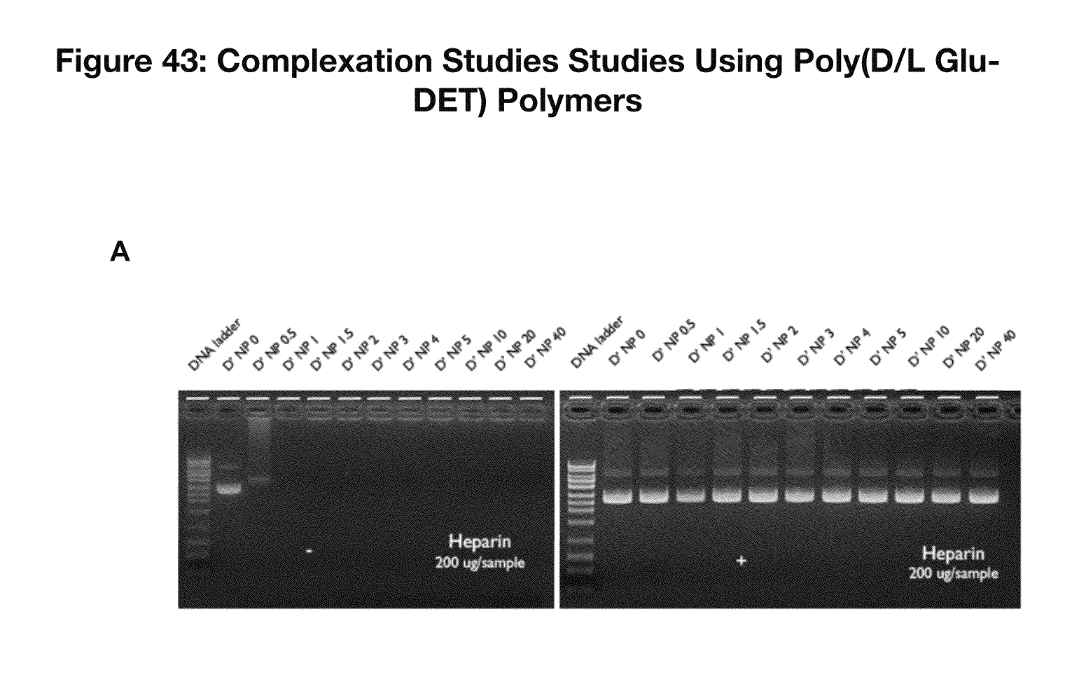

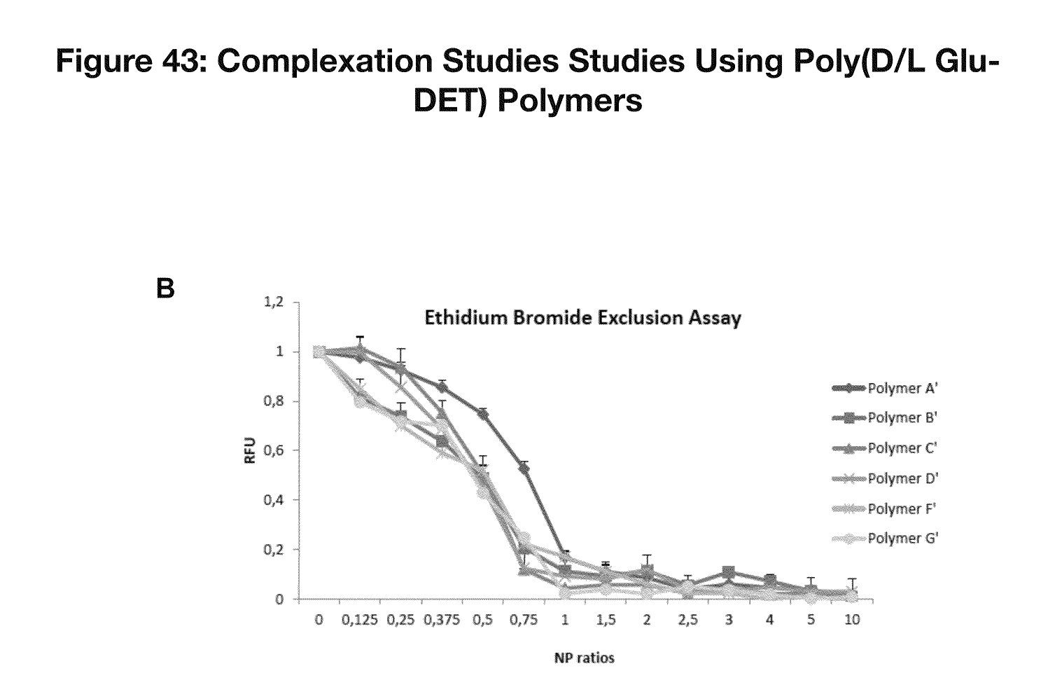

Polyplexes containing Luciferase plasmid DNA (pGL4; Promega, Madison, Wis.) were prepared (as described in Example 75) at various N:P ratios. Five μL of each formulation was run on a 1% agarose gel and visualized by ethidium bromide staining, Poly(D/L Glu-DET) polymer D, FIG. 43A. Ethidium bromide exclusion assays were performed as described previously (Example 48), FIG. 43B.

### Example 77

### Size Analysis and Morphologies of Poly(D/L Glu-DET)/DNA Polyplexes at Various N:P Ratios

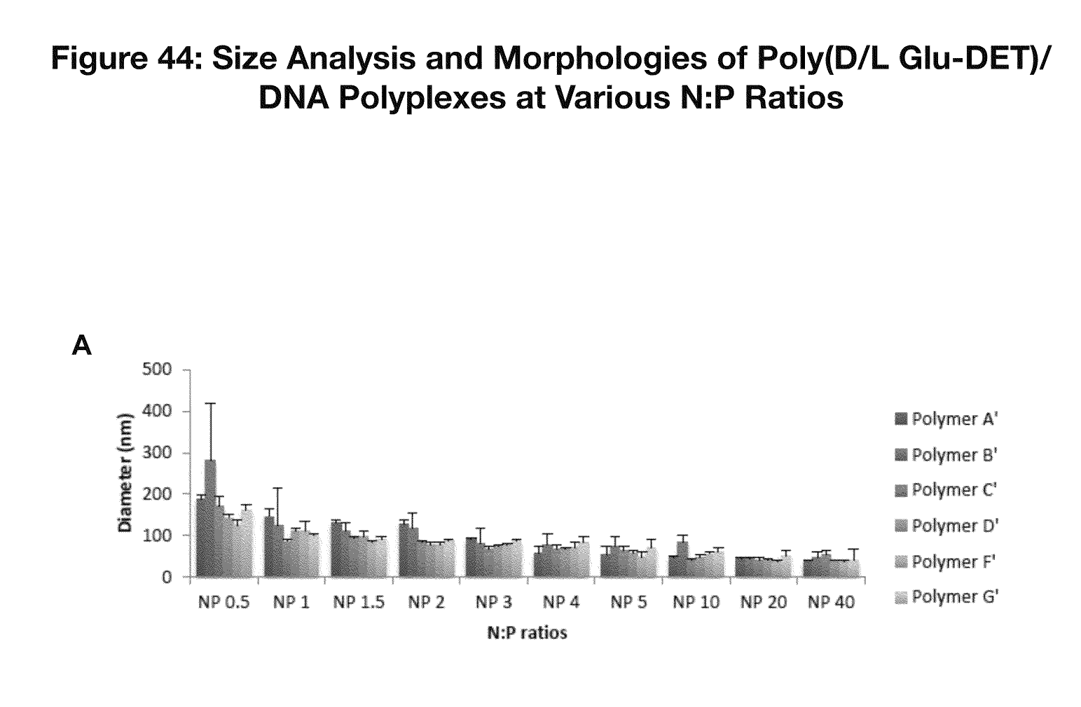

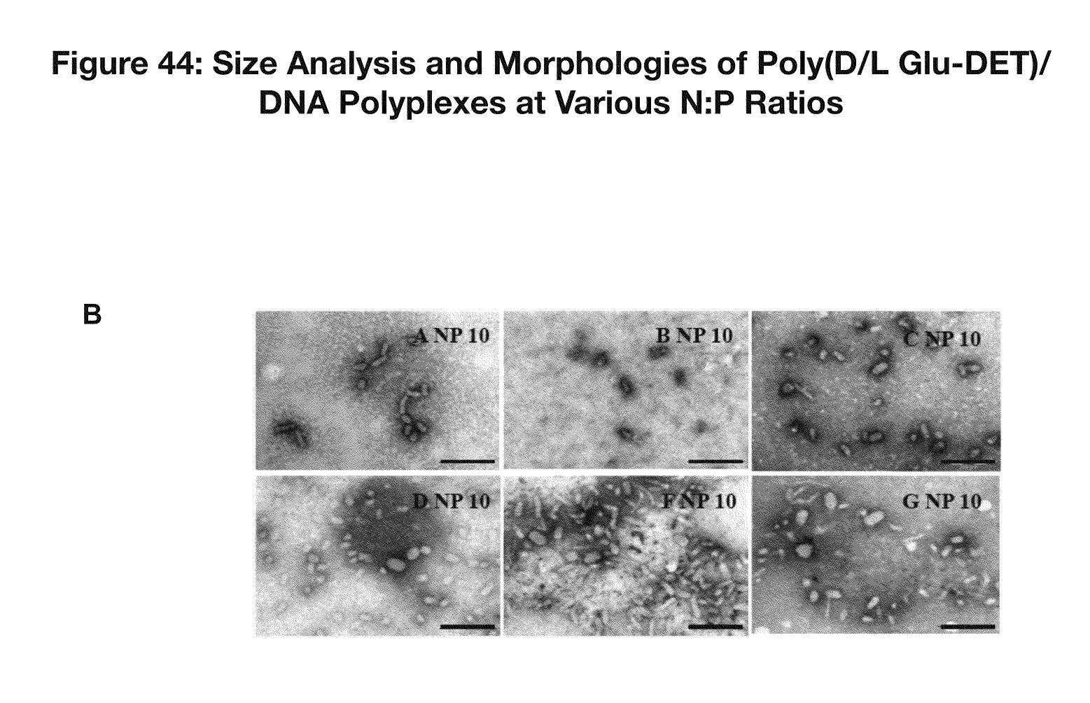

Dynamic light scattering analysis (Example 38 & 48A) of polyplex size for the Poly(D/L Glu-DET) polymers between N:P ratios of 0.5 and 40 ranged from ˜300 to ˜40 nm, FIG. 44A. TEM analysis (Example 40) of Poly(D/L Glu-DET) polymer interactions with plasmid DNA. Uniform sized structures less than 100 nm in size formed at N:P 10 ratios, FIG. 44B.

### Example 78

### DNAse Protection Assay of Poly(D/L Glu-DET)/DNA Polyplexes

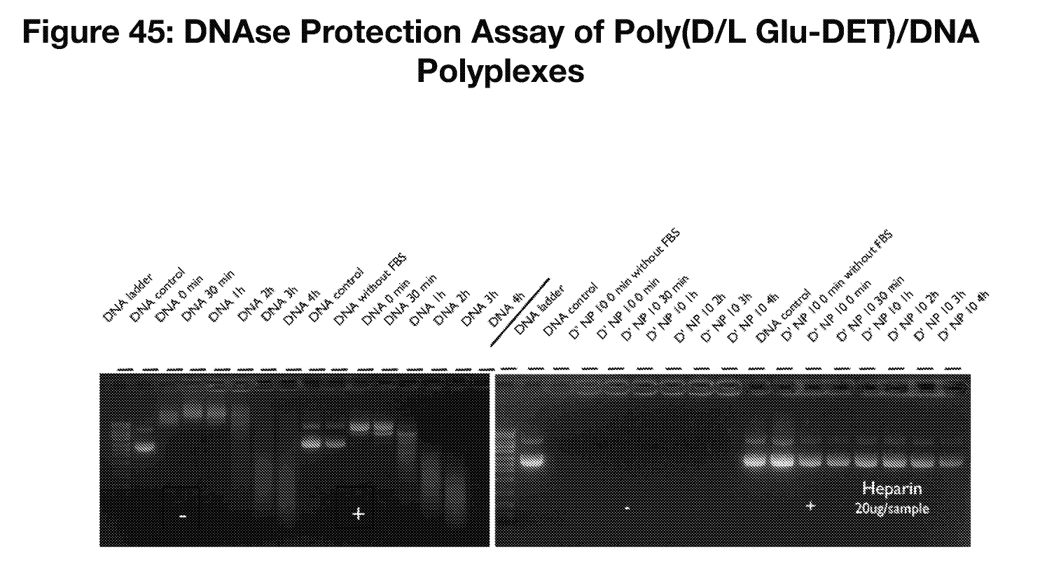

DNAse protection assaya were performed as described in Example 49. Samples were electrophoresed in a 1% agarose gel as described for the gel retardation experiment (Example 48), FIG. 45.

### Example 79

### Luciferase and GFP Expression of HCT-116 Cells Transiently Transfected with Poly(D/L Glu-DET) Polymers

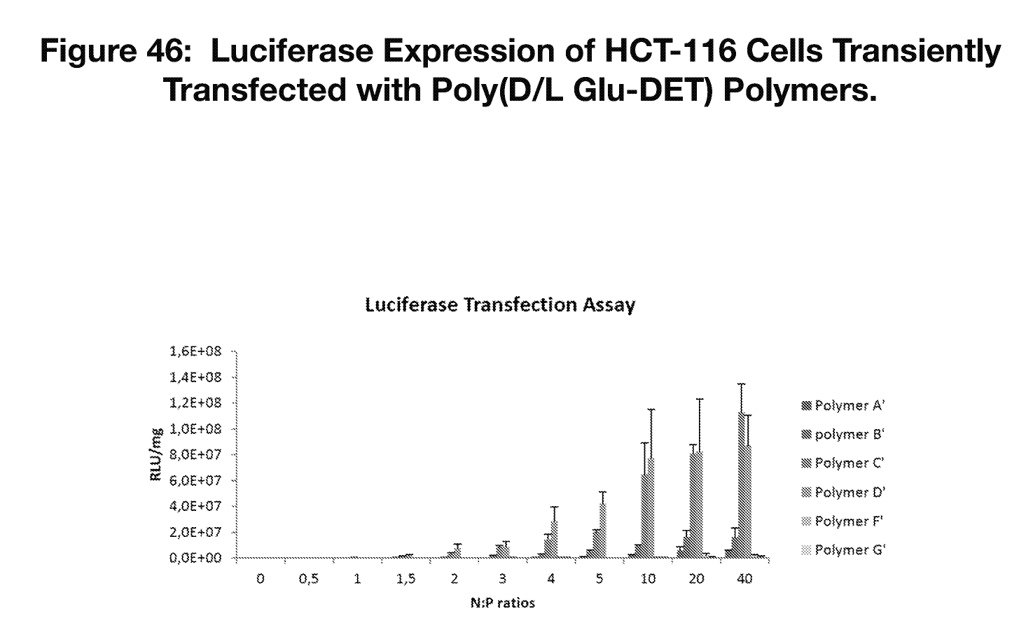

HCT-116 cells were transfected in triplicate in 96-well plates with Poly(D/L Glu-DET)/DNA polyplexes (Example 75), containing the firefly luciferase pGL4 plasmid DNA, at the indicated N:P ratios and a final DNA concentration of 0.25 μg per well. Twenty-four hr after transfection, luciferase activity for each sample was determined and was normalized to protein content. All results are representative of triplicate experiments. Luciferase activity for various sized D/L mix configurations increased with increasing N:P ratios, FIG. 46.

### Example 80

### Comparison of Polyplex and PEG-Polyplex DNA Complexation Ability

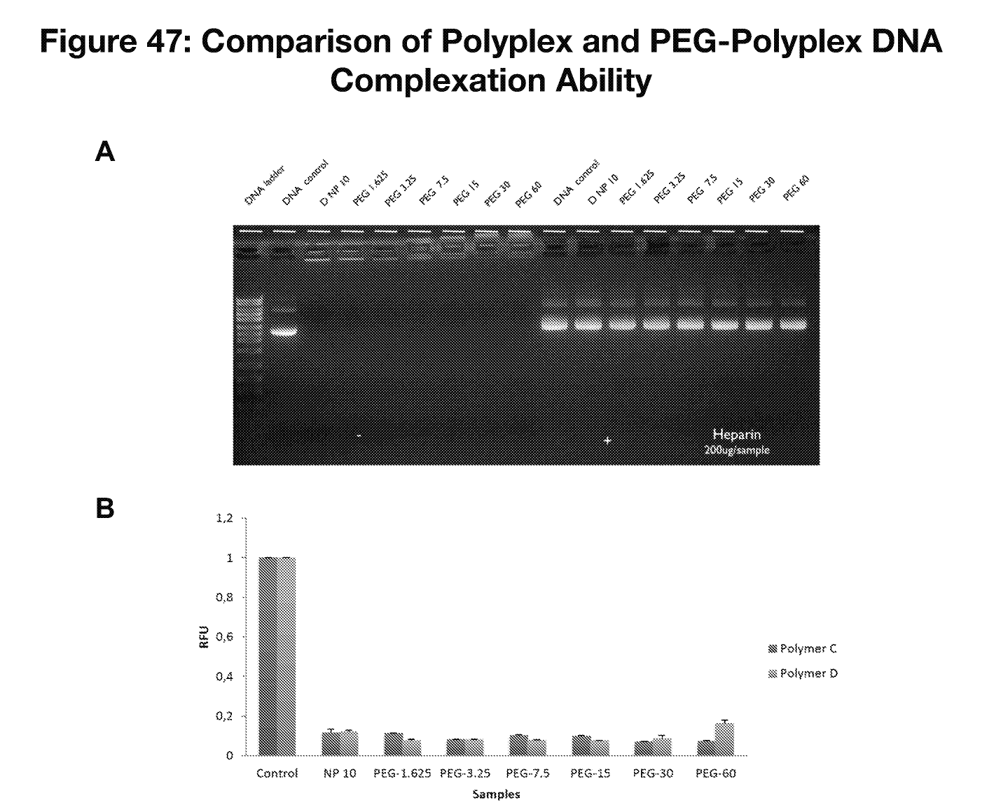

Agarose gel retardation of Poly(D/L Glu-DET) Polymer D/DNA Polyplexes at N:P 10 ratio. Samples were resolved on a 1% agarose gel and visualized by ethidium bromide, FIG. 47A. Both Polyplex and PEG-Polyplexes fully complexed 20 μg of plasmid DNA, and the addition of heparin demonstrated that all samples contained intact DNA. The relative binding affinity of Poly(D/L Glu-DET) Polyplexes or PEG-Polyplexes for plasmid DNA was measured by ethidium bromide fluorescence quenching (Example 48), FIG. 47B. Data are the average ±SD (n=3). Addition of PEG to Polyplexes had minimal effect on Poly(D/L Asp-DET) polymer binding affinity to DNA.

### Example 81

### Comparison of Polyplex and PEG-Polyplex Size and Morphology

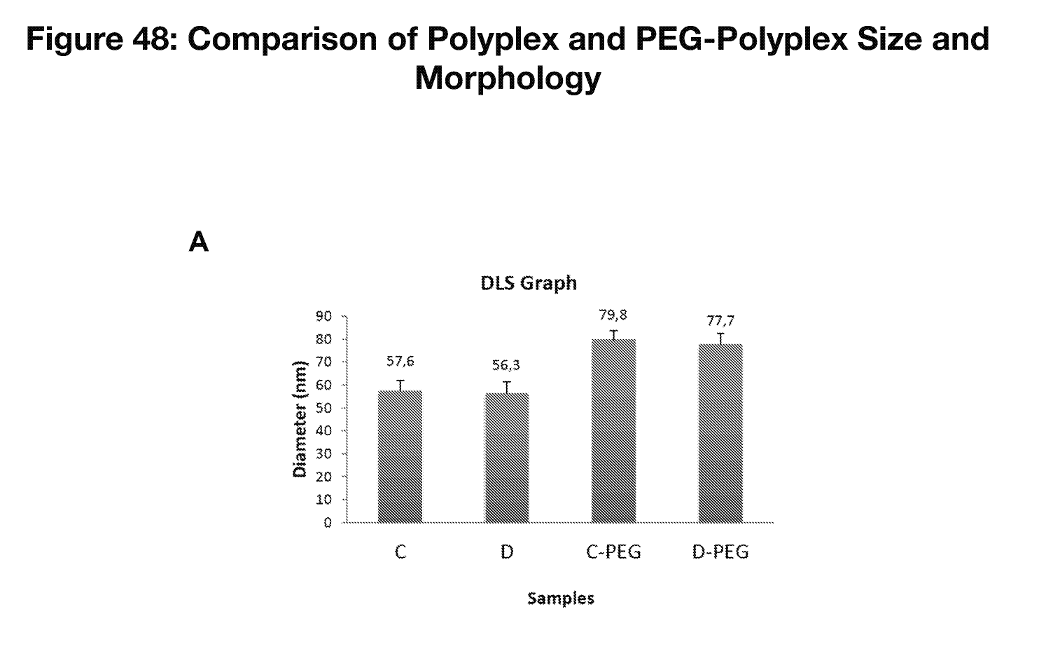

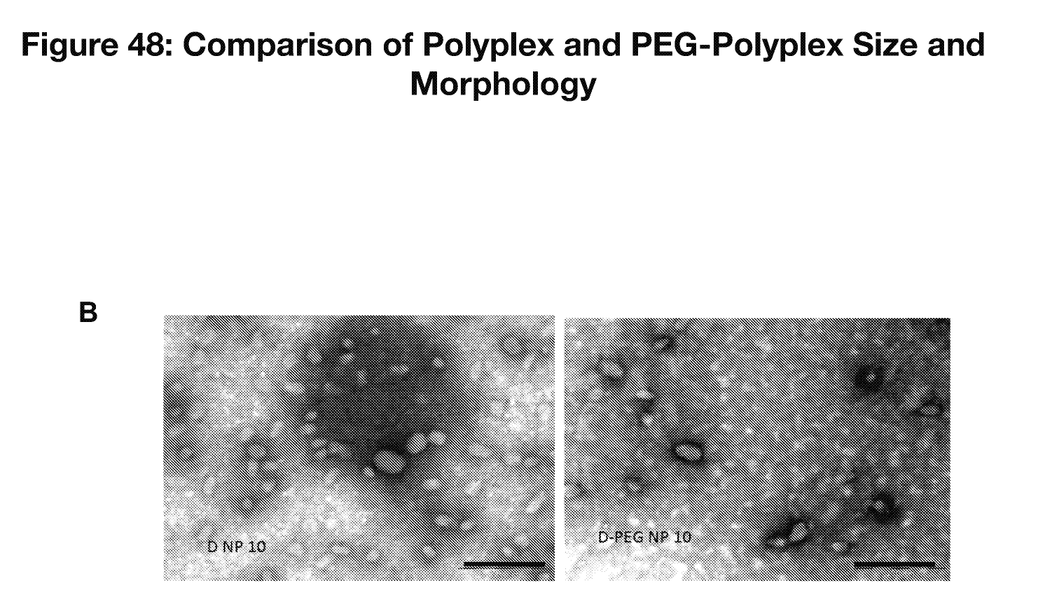

DLS analysis of Poly(D/L Glu-DET) Polyplexes and PEG-Polyplexes at N:P 10, FIG. 48A. DLS measurement determined Poly(D/L Glu-DET) PEG-polyplexes to be approximately 20 nm larger in diameter than Poly(D/L Glu-DET) Polyplexes. TEM of Poly(D/L Glu-DET)/DNA N:P 10 Polyplexes and PEG-Polyplexes, FIG. 48B. PEG-Polyplexes showed similar morphology to polyplexes and were also smaller than 100 nm. Bar=200 nm.

### Example 82

### Salt Stability Studies of Poly(D/L Glu-DET)/DNA Polyplexes

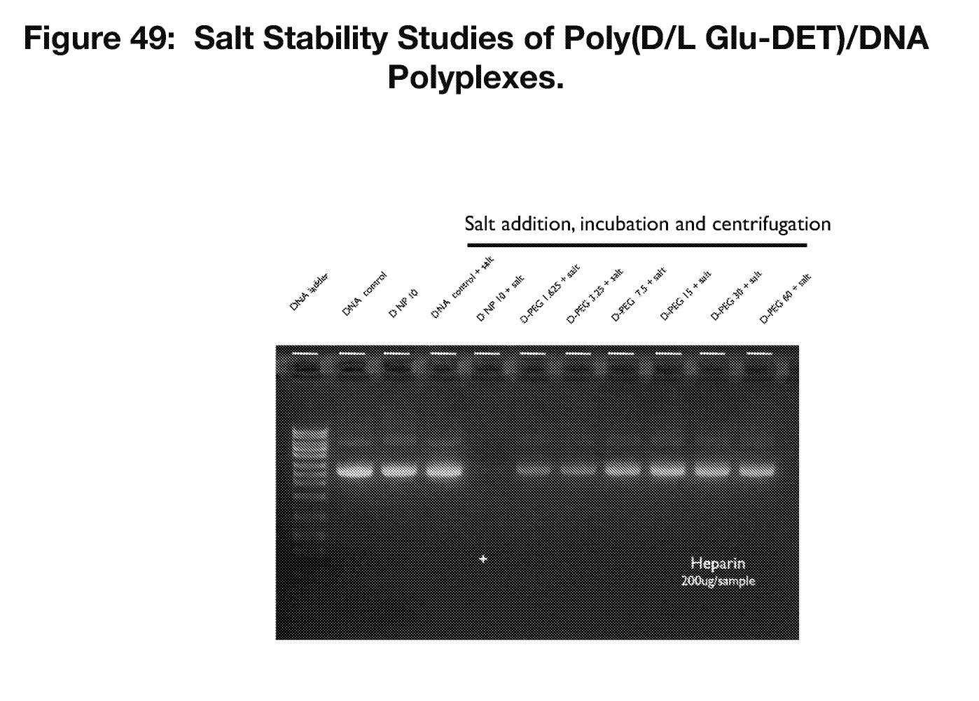

Polyplex samples were centrifuged following salt addition and incubation as described in Example 44. After centrifugation, heparin was added to supernatant to dissociate DNA from polymers and the samples were resolved on a 1% agarose/ethidium bromide gel, shown in FIG. 49. Only PEG-Polyplex samples remained in solution, and contained intact DNA following the addition of salt.

### Example 83

### Localization and Expression of Luciferase Plasmid DNA in Colon Cancer Cells Transfected with Poly(D/L Glu-DET)/DNA Polymers

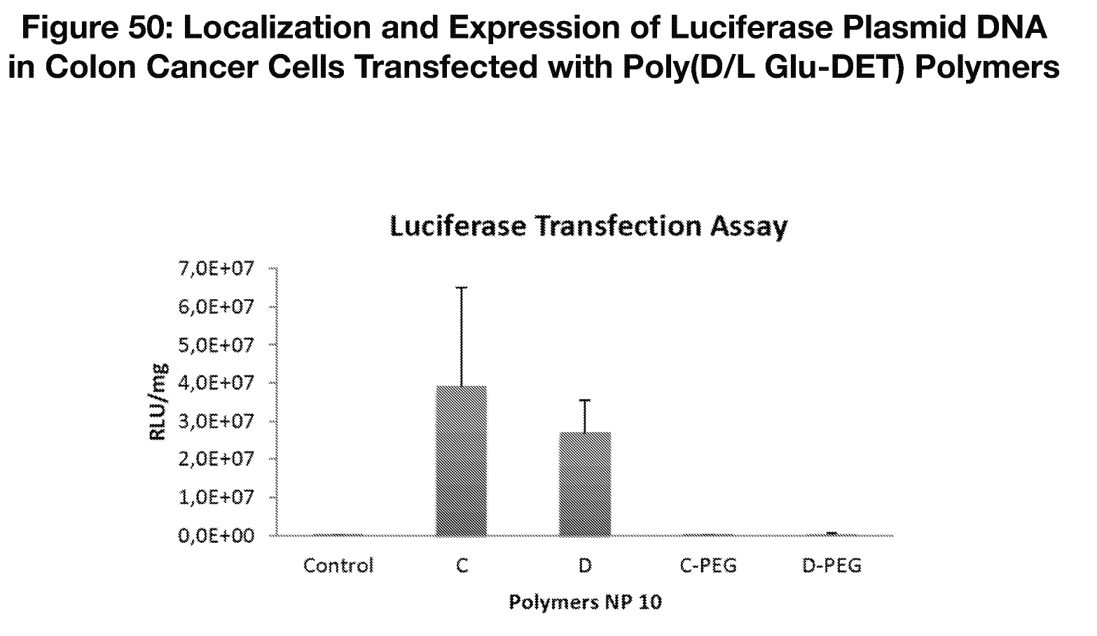

HCT-116 cells were transfected in triplicate in 96-well plates with Poly(D/L Glu-DET)/DNA polyplexes and PEG-Polyplexes (Example 75), containing firefly luciferase pGL4 plasmid DNA at a final DNA concentration of 0.25 μg DNA per well. Twenty-four hr after transfection, luciferase activity for each sample was determined and was normalized to protein content. All results are representative of triplicate experiments. Luciferase activity was only observed in cells treated with Polyplexes, FIG. 50.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A PEG-conjugated polyplex having a polynucleotide encapsulated therein, comprising a polymer of formula V or a salt thereof: 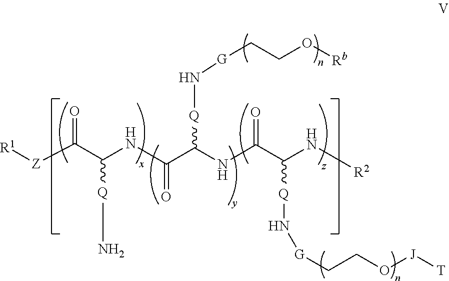
wherein:
n is 40-500;
x is 10-250;
y is 1-200;
z is 1-200;
each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH-, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—;
Z is a valence bond or a bivalent, saturated or unsaturated, straight or branched C₁₋₁₂ hydrocarbon chain, wherein 0-6 methylene units are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—;
R¹ is hydrogen, —N₃, —CN, a suitable amine protecting group, a protected aldehyde, a protected hydroxyl, a suitable hydroxyl protecting group, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide;
each R^{b} is independently —CH₃, a saturated or unsaturated alkyl moiety, an alkyne containing moiety, an azide containing moiety, a protected amine moiety, an aldehyde or protected aldehydes containing moiety, a thiol or protected thiol containing moiety, a cyclooctyne containing moiety, difluorocyclooctyne containing moiety, a nitrile oxide containing moiety, an oxanorbornadiene containing moiety, or an alcohol or protected alcohol containing moiety;
each J is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—; and,
each T is independently a targeting group.

2. The polyplex of claim 1 wherein Q is selected from —CH₂C(O)NH(CH₂)₂NH(CH₂)₂— or —(CH₂)₂C(O)NH(CH₂)₂NH(CH₂)₂.

3. The polyplex of claim 1 wherein R^{b} is —CH₂CH₂N₃.

4. The polyplex of claim 1 wherein R^{b} is —CH₃.

5. The polyplex of claim 1, wherein each polymer chain comprises a mixture of —CH₂CH₂N₃ and —CH₃ groups at the R^{b} position.

6. The polyplex of claim 1, wherein the encapsulated polynucleotide is a RNA.

7. The polyplex of claim 1, wherein the RNA is siRNA.

8. The polyplex of claim 1, wherein the encapsulated polynucleotide is a DNA.

9. The polyplex of claim 1, wherein the DNA is a plasmid DNA.

10. The polyplex of claim 1, wherein R¹ is a saturated or unsaturated C₁₋₁₂ alkyl chain.

11. The polyplex of claim 1, wherein R² is hydrogen or acyl.

12. The polyplex of claim 1, wherein R² is acetyl.

13. The polyplex of claim 1, wherein Z is —NH—.

14. The polyplex of claim 1, wherein G is a valence bond or is selected from:

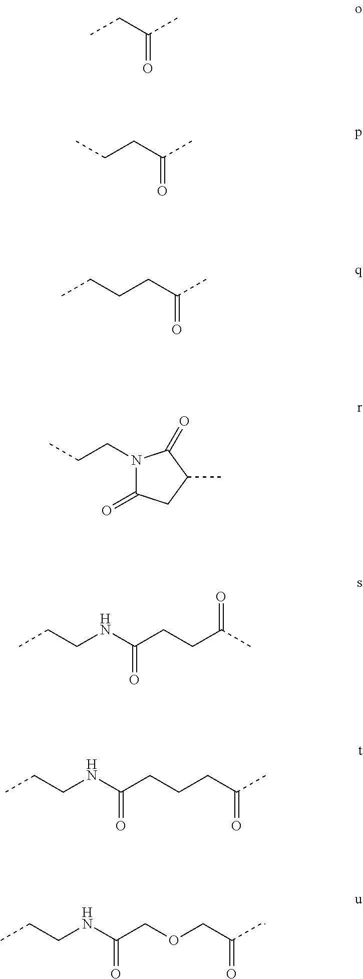

15. The polyplex of claim 1, wherein J is a valence bond, a carbonyl group, or is selected from:

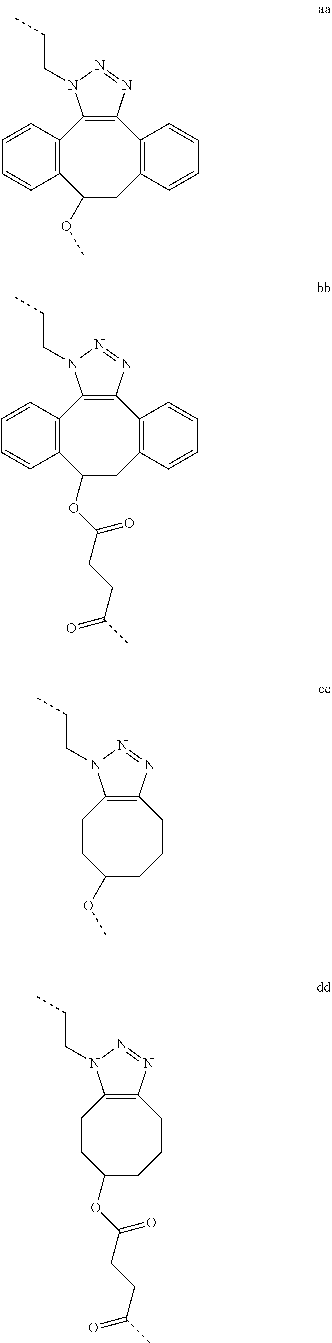

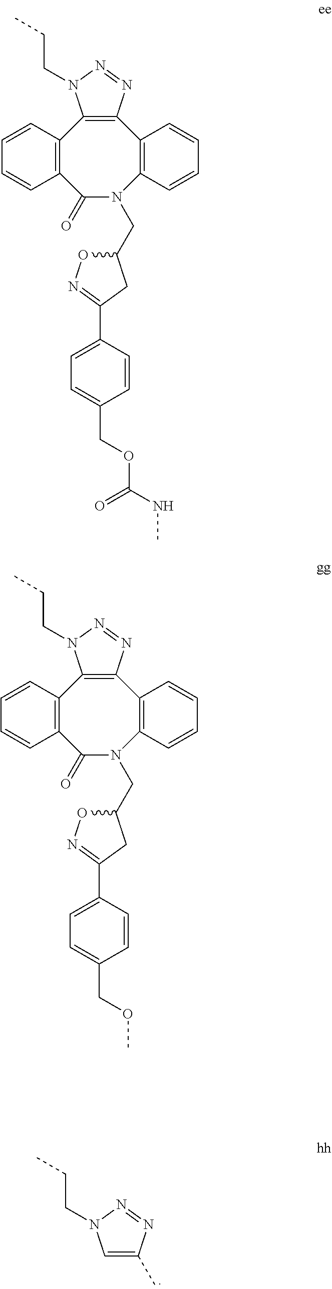

16. The polyplex of claim 1, wherein T is selected from an EGFR targeting peptide, transferrin, an EGF protein, or a fragment thereof.

17. A method comprising the steps of:
(1) providing a PEG-conjugated polyplex having a polynucleotide encapsulated therein, comprising a cationic polymer of formula III: 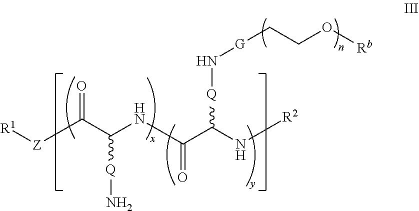
wherein:
n is 40-500;
x is 10-250;
y is 1-200;
each Q group is independently selected from a valence bond or a bivalent, saturated or unsaturated, straight or branched C₁₋₂₀ alkylene chain, wherein 0-9 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each G is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—;
Z is a valence bond or a bivalent, saturated or unsaturated, straight or branched C₁₋₁₂ hydrocarbon chain, wherein 0-6 methylene units are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—;
R¹ is hydrogen, —N₃, —CN, a suitable amine protecting group, a protected aldehyde, a protected hydroxyl, a suitable hydroxyl protecting group, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety or an oligopeptide targeting group;
R² is selected from hydrogen, an optionally substituted aliphatic group, an acyl group, a sulfonyl group, or a fusogenic peptide; and
each R^{b} is independently —CH₃, a saturated or unsaturated alkyl moiety, an alkyne containing moiety, an azide containing moiety, a protected amine moiety, an aldehyde or protected aldehydes containing moiety, a thiol or protected thiol containing moiety, a cyclooctyne containing moiety, difluorocyclooctyne containing moiety, a nitrile oxide containing moiety, an oxanorbornadiene containing moiety, or an alcohol or protected alcohol containing moiety; and
(2) performing a Click reaction between the R^{b} group of formula III with a suitable click-ready targeting group to provide a targeted, PEG-conjugated polyplex of Formula V: 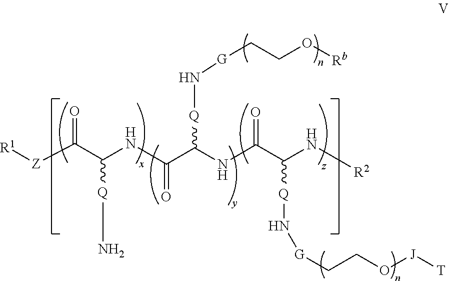
wherein:
each J is independently a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—; and,
each T is independently a targeting group.

18. A composition comprising the polyplex according to claim 1, and a pharmaceutically acceptable carrier or vehicle.

19. The composition according to claim 18, formulated for parenteral administration.
